(19) United States
(12) Patent Application Publication
(10) Pub. No.: US 2025/0297294 A1
(43) Pub. Date: Sep. 25, 2025

(54) COMPOSITIONS AND METHODS FOR IMPROVED PRODUCTION OF HUMAN MILK OLIGOSACCHARIDES

(71) Applicant: Amyris, Inc., Emeryville, CA (US)

(72) Inventors: Dominic PINEL, Emeryville, CA (US); Jessica WALTER, Emeryville, CA (US); Jacqueline T. HUMPHRIES, Emeryville, CA (US); Jason BRICE, Emeryville, CA (US); Nikita JAYANT KUMAR OJHA, Emeryville, CA (US); Christopher F. MUGLER, Emeryville, CA (US)

(21) Appl. No.: 18/722,302

(22) PCT Filed: Dec. 22, 2022
(86) PCT No.: PCT/US2022/053805

§ 371 Date: Jun. 20, 2024

Related U.S. Application Data
(60) Provisional application No. 63/293,416, filed on Dec. 23, 2021.

(51) Int. Cl. C12P 19/04; C07H 1/00; C12N 9/04; C12N 9/10; C12N 9/12; C12N 9/88; C12N 15/81; C12P 19/12; C12P 19/18

(52) U.S. Cl. CPC C12P 19/04; C07H 1/00; C12N 9/0006; C12N 9/1051; C12N 9/1205; C12N 9/88; C12N 15/81; C12P 19/12; C12P 19/18; C12Y 101/01271; C12Y 402/01047; C12N 2800/102

(57) ABSTRACT

Provided herein are host cells capable of producing a human milk oligosaccharide (HMO), such as yeast cells that include one or more heterologous nucleic acids encoding one or more enzymes of the HMO biosynthetic pathway, such as a fucosyltransferase, GDP-mannose dehydratase, lactose permease, and/or fucose synthase. Also provided are fermentation compositions including the disclosed host cells, as well as related methods of producing and recovering HMOs generated by the host cells.

### SEQUENCE LISTING

The instant application contains a Sequence Listing which has been submitted electronically in XML format and is hereby incorporated by reference in its entirety. Said XML copy, created on Apr. 18, 2025, is named 51494-019002_Sequence_Listing_4_18_25.xml and is 154,744 bytes in size.

### BACKGROUND OF THE INVENTION

Human milk oligosaccharides (HMOs) are the third most abundant component of human milk, with only lactose and lipids present in higher concentrations. More than 200 different species of HMOs have been identified to date in human milk, including the naturally occurring tetra-saccharide lacto-n-neotetraose (LNnT), belonging to the group of non-fucosylated neutral HMOs, and 2′-fucosyllactose (2′-FL). There is growing evidence attributing various health benefits to these milk compounds. Exemplary benefits include the promotion of the growth of protective intestinal microbes such as bifidobacteria, an increase in protection from gastrointestinal infections, a strengthening of the immune system, and an improvement in cognitive development. Because HMOs are not found in other milk sources, such as cow or goat, the only source of HMOs has traditionally been mother's milk. In efforts to improve the nutritional value of infant formula and expand the use of HMOs for child and adult nutrition, there has been an increased interest in the synthetic production of these compounds.

Heterologous production of 2′-FL in yeast requires four non-native enzymes: fucosyltransferase, lactose permease to import fed lactose, GDP-mannose 4,6-dehydratase, and fucose synthase. Generation of unwanted byproducts, however, is a significant challenge to the manufacturing of 2′-FL and other HMOs. Therefore, there remains a need for improved methods that result in enhanced HMO production and fewer unwanted byproducts.

### SUMMARY OF THE INVENTION

The present disclosure provides host cells that are capable of producing a human milk oligosaccharide (HMO) and that have been genetically modified to express one or more heterologous nucleic acids that encode an enzyme of the biosynthetic pathway for the corresponding HMO. The disclosure also features particular biosynthetic enzymes useful for producing certain HMOs, as well as nucleic acids encoding such enzymes. For example, the disclosure provides a series of fucosyltransferase, GDP-mannose dehydratase (GMD), lactose permease, and fucose synthase polypeptides, nucleic acids encoding the same, and host cells expressing such polypeptides, as well as methods of using these compositions to produce a HMO in a host cell, such as a yeast cell.

The enzymes described herein exhibit a series of advantageous biochemical properties, as these polypeptides have presently been discovered to produce desired intermediates in a HMO biosynthetic pathway with high selectivity and catalytic efficiency. This, in turn, provides the benefit of allowing for the production of a given HMO with high purity and titer. The sections that follow describe, in further detail, the various polypeptides of the disclosure and how host cells encoding one or more of these polypeptides may be used to produce a desired HMO.

In an aspect, the disclosure provides a host cell capable of producing a HMO. The host cell may contain one or more heterologous nucleic acids encoding one or more enzymes of the HMO biosynthetic pathway. For example, the host cell may contain one or more heterologous nucleic acids that each, independently, encode: a fucosyltransferase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 1-41; and/or a GMD having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 42-64; and/or a lactose permease having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 65-99; and/or a fucose synthase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 100-103.

In another aspect, the disclosure provides a host cell capable of producing a HMO, wherein the host cell includes one or more heterologous nucleic acids that each, independently, encode a fucosyltransferase, a GMD, a lactose permease, and/or a fucose synthase. In some embodiments, the host cell produces the HMO at a yield of at least 20% (w/w). In some embodiments, the host cell produces the HMO at a yield of between 20% (w/w) and 70% (w/w) (e.g., between 20% % (w/w) and 60% (w/w), 20% (w/w) and 50% (w/w), 20% (w/w) and 40% (w/w), 20% (w/w) and 30% (w/w), 30% (w/w) and 70% (w/w), 40% (w/w) and 70% (w/w), 50% (w/w) and 70% (w/w), or 60% (w/w) and 70% (w/w)). In some embodiments, the host cell produces the HMO at a yield of between 40% (w/w) and 70% (w/w) (e.g., between 50% (w/w) and 70% (w/w), 60% (w/w) and 70% (w/w), 40% (w/w) and 60% (w/w), or 40% (w/w) and 50% (w/w)).

In another aspect, the disclosure provides a host cell capable of producing a HMO, wherein the host cell includes one or more heterologous nucleic acids that each, independently, encode a fucosyltransferase, a GMD, a lactose permease, and/or a fucose synthase. In some embodiments, the host cell produces the HMO at a productivity of at least 1 g/L/hr. In some embodiments, the host cell produces the HMO at a productivity of between 1 g/L/hr and 5 g/L/hr (e.g., between 1 g/L/hr and 4 g/L/hr, 1 g/L/hr and 3 g/L/hr, 1 g/L/hr and 2 g/L/hr, 2 g/L/hr and 5 g/L/hr, 3 g/L/hr and 5 g/L/hr, or 4 g/L/hr and 5 g/L/hr). In some embodiments, the host cell produces the HMO at a productivity of between 2 g/L/hr and 5 g/L/hr (e.g., between 2 g/L/hr and 3 g/L/hr, 2 g/L/hr and 4 g/L/hr, 3 g/L/hr and 5 g/L/hr, 3 g/L/hr and 5 g/L/hr, or 3 g/L/hr and 4 g/L/hr).

In some embodiments, the host cell includes a heterologous nucleic acid that encodes a fucosyltransferase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 1-41. In some embodiments, the host cell includes a heterologous nucleic acid that encodes a fucosyltransferase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 1-41. In some embodiments, the fucosyltransferase has an amino acid sequence that is at least 95% identical (e.g., at least 95%, 96%, 97%, 98%, 99%, or 100% identical) to the amino acid sequence of any one of SEQ ID NOS: 1-41. In some embodiments, the fucosyltransferase has the amino acid sequence of any one of SEQ ID NOS: 1-41.

In some embodiments, the host cell includes a heterologous nucleic acid that encodes a fucosyltransferase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 1-3 and 6-41. In some embodiments, the fucosyltransferase has an amino acid sequence that is at least 95% identical (e.g., at least 95%, 96%, 97%, 98%, 99%, or 100% identical) to the amino acid sequence of any one of SEQ ID NOS: 1-3 and 6-41. In some embodiments, the fucosyltransferase has the amino acid sequence of any one of SEQ ID NOS: 1-3 and 6-41.

In some embodiments, the host cell includes a heterologous nucleic acid that encodes a fucosyltransferase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 1-3. In some embodiments, the fucosyltransferase has an amino acid sequence that is at least 95% identical (e.g., at least 95%, 96%, 97%, 98%, 99%, or 100% identical) to the amino acid sequence of any one of SEQ ID NOS: 1-3. In some embodiments, the fucosyltransferase has the amino acid sequence of any one of SEQ ID NOS: 1-3.

In some embodiments, the host cell includes a heterologous nucleic acid that encodes a GMD having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 42-64. In some embodiments, the host cell includes a heterologous nucleic acid that encodes a GMD having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 42-64. In some embodiments, the GMD has an amino acid sequence that is at least 95% identical (e.g., at least 95%, 96%, 97%, 98%, 99%, or 100% identical) to the amino acid sequence of any one of SEQ ID NOS: 42-64. In some embodiments, the GMD has the amino acid sequence of any one of SEQ ID NOS: 42-64.

In some embodiments, the host cell includes a heterologous nucleic acid that encodes a GMD having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 42-44. In some embodiments, the GMD has an amino acid sequence that is at least 95% identical (e.g., at least 95%, 96%, 97%, 98%, 99%, or 100% identical) to the amino acid sequence of any one of SEQ ID NOS: 42-44. In some embodiments, the GMD has the amino acid sequence of any one of SEQ ID NOS: 42-44.

In some embodiments, the host cell includes a heterologous nucleic acid that encodes a lactose permease having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 65-99. In some embodiments, the host cell includes a heterologous nucleic acid that encodes a lactose permease having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 65-99. In some embodiments, the lactose permease has an amino acid sequence that is at least 95% identical (e.g., at least 95%, 96%, 97%, 98%, 99%, or 100% identical) to the amino acid sequence of any one of SEQ ID NOS: 65-99. In some embodiments, the lactose permease has the amino acid sequence of any one of SEQ ID NOS: 65-99.

In some embodiments, the host cell includes a heterologous nucleic acid that encodes a fucose synthase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 100-103. In some embodiments, the host cell includes a heterologous nucleic acid that encodes a fucose synthase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 100-103. In some embodiments, the fucose synthase has an amino acid sequence that is at least 95% identical (e.g., at least 95%, 96%, 97%, 98%, 99%, or 100% identical) to the amino acid sequence of any one of SEQ ID NOS: 100-103. In some embodiments, the fucose synthase has the amino acid sequence of any one of SEQ ID NOS: 100-103.

In some embodiments, the one or more heterologous nucleic acids are integrated into the genome of the host cell. In some embodiments, the one or more heterologous nucleic acids are present within one or more plasmids in the host cell.

In some embodiments, the HMO is a reducing sugar. In some embodiments, the HMO includes a fucose residue. The HMO may be, for example, lacto-N-neotetraose (LNnT), 2′-fucosyllactose (2′-FL), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, or fucosyl-disialyllacto-N-hexaose (FDS-LNH) II.

In some embodiments, the host cell further includes (e.g., expresses) one or more of a β-1,3-N-acetylglucosaminyltransferase (LgtA), a β-1,4-galactosyltransferase (LgtB), and a UDP-N-acetylglucosamine diphosphorylase. In some embodiments, the host cell further includes a LgtA. In some embodiments, the LgtA has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 104. In some embodiments, the LgtA has an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 104. In some embodiments, the LgtA has an amino acid sequence that is at least 95% identical (e.g., at least 95%, 96%, 97%, 98%, 99%, or 100% identical) to the amino acid sequence of SEQ ID NO: 104. In some embodiments,
the LgtA has the amino acid sequence of SEQ ID NO: 104.

In some embodiments, the LgtA includes one or more amino acid substitutions or deletions relative to the amino acid sequence of SEQ ID NO: 104. In some embodiments, the LgtA has an amino acid sequence that is from about 85% to about 99.7% identical to the amino acid sequence of SEQ ID NO: 104, optionally wherein the LgtA has an amino acid sequence that is from about 90% to about 99.7% identical to the amino acid sequence of SEQ ID NO: 104, optionally wherein the LgtA has an amino acid sequence that is from about 95% to about 99.7% identical to the amino acid sequence of SEQ ID NO: 104. In some embodiments, the LgtA has an amino acid sequence that differs from the amino acid sequence of SEQ ID NO: 104 only by way of (i) the one or more amino acid substitutions or deletions and, optionally, (ii) one or more additional, conservative amino acid substitutions. In some embodiments,
the LgtA has an amino acid sequence that differs from the amino acid sequence of SEQ ID NO: 104 only by way of the one or more amino acid substitutions or deletions.

In some embodiments, the LgtA has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 105-120. In some embodiments, the LgtA has an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 105-120. In some embodiment, the LgtA has an amino acid sequence that is at least 95% identical (e.g., at least 95%, 96%, 97%, 98%, 99%, or 100% identical) to the amino acid sequence of any one of SEQ ID NOS: 105-120. In some embodiments, the LgtA has the amino acid sequence of any one of SEQ ID NOS: 105-120.

In some embodiments, the host cell further includes (e.g., expresses) a LgtB. In some embodiments, the LgtB has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 121. In some embodiments, the LgtB has an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 121. In some embodiments, the LgtB has an amino acid sequence that is at least 95% identical (e.g., at least 95%, 96%, 97%, 98%, 99%, or 100% identical) to the amino acid sequence of SEQ ID NO: 121. In some embodiments, the LgtB has the amino acid sequence of SEQ ID NO: 121.

In some embodiments, the LgtB has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 122. In some embodiments, the LgtB has an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 122. In some embodiments, the LgtB has an amino acid sequence that is at least 95% identical (e.g., at least 95%, 96%, 97%, 98%, 99%, or 100% identical) to the amino acid sequence of SEQ ID NO: 122. In some embodiments, the LgtB has the amino acid sequence of SEQ ID NO: 122.

In some embodiments, the HMO is 2′-FL. In some embodiments, the HMO is 6′-SL. In some embodiments, the host cell further comprises a heterologous nucleic acid encoding one or more of PSA, SEC53, HEM12, SAK1, ACS1, DAN1, or NYP1 proteins. In some embodiments, any one of the heterologous nucleic acids are not under the control of a maltose-responsive promoter.

In some embodiments, the host cell further includes (e.g., expresses) a fucosidase. In some embodiments, the host cell further includes (e.g., expresses) a protein that transports lactose into the host cell. In some embodiments, the protein that transports lactose into the cell is an active transporter.

In some embodiments, expression of the one or more heterologous nucleic acids is driven by an inducible promoter or is negatively regulated by the activity of a promoter that is responsive to a small molecule.

In some embodiments, the host cell produces the HMO at a yield of 20% (w/w). In some embodiments, the host cell produces the HMO at a yield of between 20% (w/w) and 70% (w/w) (e.g., between 20% % (w/w) and 60% (w/w), 20% (w/w) and 50% (w/w), 20% (w/w) and 40% (w/w), 20% (w/w) and 30% (w/w), 30% (w/w) and 70% (w/w), 40% (w/w) and 70% (w/w), 50% (w/w) and 70% (w/w), or 60% (w/w) and 70% (w/w)). In some embodiments, In some embodiments, the host cell produces the HMO at a yield of between 40% (w/w) and 70% (w/w) (e.g., between 50% (w/w) and 70% (w/w), 60% (w/w) and 70% (w/w), 40% (w/w) and 60% (w/w), or 40% (w/w) and 50% (w/w)). In some embodiments the host cell produces the HMO at a productivity of at least 1 g/L/hr. In some embodiments, the host cell produces the HMO at a productivity of 1 g/L/hr and 5 g/L/hr (e.g., between 1 g/L/hr and 4 g/L/hr, 1 g/L/hr and 3 g/L/hr, 1 g/L/hr and 2 g/L/hr, 2 g/L/hr and 5 g/L/hr, 3 g/L/hr and 5 g/L/hr, or 4 g/L/hr and 5 g/L/hr). In some embodiments, the host cell produces the HMO at a productivity of between 2 g/L/hr and 5 g/L/hr (e.g., between 2 g/L/hr and 3 g/L/hr, 2 g/L/hr and 4 g/L/hr, 3 g/L/hr and 5 g/L/hr, 3 g/L/hr and 5 g/L/hr, or 3 g/L/hr and 4 g/L/hr).

In some embodiments, the host cell is a yeast cell. In some embodiments, the yeast cell is a Saccharomyces sp. cell or a Kluveromyces sp. cell. In some embodiments, the yeast cell is a Saccharomyces cerevisiae cell. In some embodiments, the yeast cell is a Kluveromyces marxianus cell.

In another aspect, the disclosure provides a method of producing a HMO including culturing a population of any one of the host cells described herein in a culture medium under conditions suitable for the host cells to produce the HMO.

In another aspect, the disclosure provides a method of genetically modifying a host cell so as to render the host cell capable of producing a HMO. The method may include, for example, introducing into the host cell one or more heterologous nucleic acids that each, independently, encode: a fucosyltransferase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 1-41; and/or a GMD having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 42-64; and/or a lactose permease having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 65-99; and/or a fucose synthase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 100-103.

In some embodiments, the method includes introducing into the host cell a heterologous nucleic acid that encodes a fucosyltransferase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 1-41. In some embodiments, the fucosyltransferase has an amino acid sequence that is at least 95% identical (e.g., at least 95%, 96%, 97%, 98%, 99%, or 100% identical) to the amino acid sequence of any one of SEQ ID NOS: 1-41. In some embodiments, the fucosyltransferase has the amino acid sequence of any one of SEQ ID NOS: 1-41.

In some embodiments, the method includes introducing into the host cell a heterologous nucleic acid that encodes a fucosyltransferase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 1-3 and 6-41. In some embodiments, the fucosyltransferase has an amino acid sequence that is at least 95% identical (e.g., at least 95%, 96%, 97%, 98%, 99%, or 100% identical) to the amino acid sequence of any one of SEQ ID NOS: 1-3 and 6-41. In some embodiments, the fucosyltransferase has the amino acid sequence of any one of SEQ ID NOS: 1-3 and 6-41.

In some embodiments, the method includes introducing into the host cell a heterologous nucleic acid that encodes a fucosyltransferase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 1-3. In some embodiments, the fucosyltransferase has an amino acid sequence that is at least 95% identical (e.g., at least 95%, 96%, 97%, 98%, 99%, or 100% identical) to the amino acid sequence of any one of SEQ ID NOS: 1-3. In some embodiments, the fucosyltransferase has the amino acid sequence of any one of SEQ ID NOS: 1-3.

In some embodiments, the method includes introducing into the host cell a heterologous nucleic acid that encodes a GMD having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 42-64. In some embodiments, the GMD has an amino acid sequence that is at least 95% identical (e.g., at least 95%, 96%, 97%, 98%, 99%, or 100% identical) to the amino acid sequence of any one of SEQ ID NOS: 42-64. In some embodiments, the GMD has the amino acid sequence of any one of SEQ ID NOS: 42-64. In some embodiments, the method includes introducing into the host cell a heterologous nucleic acid that encodes a GMD having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 42-44. In some embodiments, the GMD has an amino acid sequence that is at least 95% identical (e.g., at least 95%, 96%, 97%, 98%, 99%, or 100% identical) to the amino acid sequence of any one of SEQ ID NOS: 42-44. In some embodiments, the GMD has the amino acid sequence of any one of SEQ ID NOS: 42-44.

In some embodiments, the method includes introducing into the host cell a heterologous nucleic acid that encodes a lactose permease having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 65-99. In some embodiments, the lactose permease has an amino acid sequence that is at least 95% identical (e.g., at least 95%, 96%, 97%, 98%, 99%, or 100% identical) to the amino acid sequence of any one of SEQ ID NOS: 65-99. In some embodiments, the lactose permease has the amino acid sequence of any one of SEQ ID NOS: 65-99.

In some embodiments, the method includes introducing into the host cell a heterologous nucleic acid that encodes a fucose synthase having an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 100-103. In some embodiments, the fucose synthase has an amino acid sequence that is at least 95% identical (e.g., at least 95%, 96%, 97%, 98%, 99%, or 100% identical) to the amino acid sequence of any one of SEQ ID NOS: 100-103. In some embodiments, the fucose synthase has the amino acid sequence of any one of SEQ ID NOS: 100-103.

In some embodiments of the foregoing methods, the one or more heterologous nucleic acids are integrated into the genome of the host cell. In some embodiments, the one or more heterologous nucleic acids are present within one or more plasmids in the host cell.

In some embodiments, the HMO is a reducing sugar. In some embodiments, the HMO includes a fucose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

In some embodiments, the method includes introducing into the host cell one or more nucleic acids encoding a LgtA, a LgtB, and/or a UDP-N-acetylglucosamine diphosphorylase. In some embodiments, the method includes introducing into the host cell a nucleic acid encoding a LgtA. In some embodiments, the LgtA has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 104. In some embodiments, the LgtA has an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 104. In some embodiments, the LgtA has an amino acid sequence that is at least 95% identical (e.g., at least 95%, 96%, 97%, 98%, 99%, or 100% identical) to the amino acid sequence of SEQ ID NO: 104. In some embodiments, the LgtA has the amino acid sequence of SEQ ID NO: 104.

In some embodiments, the LgtA includes one or more amino acid substitutions or deletions relative to the amino acid sequence of SEQ ID NO: 104. In some embodiments, the LgtA has an amino acid sequence that is from about 85% to about 99.7% identical to the amino acid sequence of SEQ ID NO: 104, optionally wherein the LgtA has an amino acid sequence that is from about 90% to about 99.7% identical to the amino acid sequence of SEQ ID NO: 104, optionally wherein the LgtA has an amino acid sequence that is from about 95% to about 99.7% identical to the amino acid sequence of SEQ ID NO: 104. In some embodiments, the LgtA has an amino acid sequence that differs from the amino acid sequence of SEQ ID NO: 104 only by way of (i) the one or more amino acid substitutions or deletions and, optionally, (ii) one or more additional, conservative amino acid substitutions. In some embodiments, the LgtA has an amino acid sequence that differs from the amino acid sequence of SEQ ID NO: 104 only by way of the one or more amino acid substitutions or deletions.

In some embodiments, the LgtA has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 105-120. In some embodiments, the LgtA has an amino acid sequence that is at least 90% identical (e.g., at least 90%, 91%, 92%, 93%, 94%, 95%, 96%, 97%, 98%, 99%, or 100% identical) to the amino acid sequence of any one of SEQ ID NOS: 105-120. In some embodiments, the LgtA has an amino acid sequence that is at least 95% identical (e.g., at least 95%, 96%, 97%, 98%, 99%, or 100% identical) to the amino acid sequence of any one of SEQ ID NOS: 105-120. In some embodiments, the LgtA has the amino acid sequence of any one of SEQ ID NOS: 105-120.

In some embodiments, the method includes introducing into the host cell a nucleic acid encoding a LgtB. In some embodiments, the LgtB has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 121. In some embodiments, the LgtB has an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 121. In some embodiments, the LgtB has an amino acid sequence that is at least 95% identical (e.g., at least 95%, 96%, 97%, 98%, 99%, or 100% identical) to the amino acid sequence of SEQ ID NO: 121. In some embodiments, the LgtB has the amino acid sequence of SEQ ID NO: 121. In some embodiments, the LgtB has an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of SEQ ID NO: 122. In some embodiments, the LgtB has an amino acid sequence that is at least 90% identical (e.g., at least 90%, 91%, 92%, 93%, 94%, 95%, 96%, 97%, 98%, 99%, or 100% identical) to the amino acid sequence of SEQ ID NO: 122. In some embodiments, the LgtB has an amino acid sequence that is at least 95% identical (e.g., at least 95%, 96%, 97%, 98%, 99%, or 100% identical) to the amino acid sequence of SEQ ID NO: 122. In some embodiments, the LgtB has the amino acid sequence of SEQ ID NO: 122.

In some embodiments of any of the foregoing methods, the HMO is 2′-FL. In some embodiments of any of the foregoing methods, the HMO is 6′-SL. In some embodiments, the host cell further comprises a heterologous nucleic acid encoding one or more of PSA, SEC53, HEM12, SAK1, ACS1, DAN1, or NYP1 proteins. In some embodiments, any one of the heterologous nucleic acids are not under the control of a maltose-responsive promoter.

In some embodiments, the method further includes introducing into the host cell a nucleic acid encoding a fucosidase. In some embodiments, the method further includes introducing into the host cell a nucleic acid encoding a protein that transports lactose into the host cell. In some embodiments, the protein that transports lactose into the cell is an active transporter.

In some embodiments, the host cell is capable of producing a yield of an HMO concentration of at least 20% (w/w). In some embodiments, the host cell is capable of producing yield of an HMO at a concentration of between 20% (w/w) and 70% (w/w) (e.g., between 20% % (w/w) and 60% (w/w), 20% (w/w) and 50% (w/w), 20% (w/w) and 40% (w/w), 20% (w/w) and 30% (w/w), 30% (w/w) and 70% (w/w), 40% (w/w) and 70% (w/w), 50% (w/w) and 70% (w/w), or 60% (w/w) and 70% (w/w)). In some embodiments, the host cell is capable of producing yield of an HMO at a concentration of between 40% (w/w) and 70% (w/w) (e.g., between 50% (w/w) and 70% (w/w), 60% (w/w) and 70% (w/w), 40% (w/w) and 60% (w/w), or 40% (w/w) and 50% (w/w)). In some embodiments the host cell produces the HMO at a productivity of at least 1 g/L/hr. In some embodiments, the host cell produces the HMO at a productivity of between 1 g/L/hr and 5 g/L/hr (e.g., between 1 g/L/hr and 4 g/L/hr, 1 g/L/hr and 3 g/L/hr, 1 g/L/hr and 2 g/L/hr, 2 g/L/hr and 5 g/L/hr, 3 g/L/hr and 5 g/L/hr, or 4 g/L/hr and 5 g/L/hr). In some embodiments, the host cell produces the HMO at a productivity of between 2 g/L/hr and 5 g/L/hr (e.g., between 2 g/L/hr and 3 g/L/hr, 2 g/L/hr and 4 g/L/hr, 3 g/L/hr and 5 g/L/hr, 3 g/L/hr and 5 g/L/hr, or 3 g/L/hr and 4 g/L/hr).

In some embodiments, the host cell is a yeast cell. In some embodiments, the yeast cell is a Saccharomyces sp. cell or a Kluveromyces sp. cell. In some embodiments, the yeast cell is a Saccharomyces cerevisiae cell. In some embodiments, the yeast cell is a Kluveromyces marxianus cell.

In another aspect, the disclosure provides a fermentation composition including a population of any one of the host cells described herein and a culture medium including a HMO produced from the host cells. In some embodiments, the HMO includes a fucose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II. In some embodiments, the HMO is 2′FL. In some embodiments, the HMO is 6′SL.

### Definitions

As used herein in the context of a protein of interest, the term “activity” refers to the biological functionality that is associated with a wild-type form of the protein. For example, in the context of an enzyme, the term “activity” may refer to the ability of an enzyme to catalyze the conversion of a substrate into a product. The activity of the enzyme may be measured, for example, by determining the amount of product in a chemical reaction after a certain period of time, and/or by determining the amount of substrate remaining in the reaction mixture after a certain period of time. The activity of the enzyme can also be measured by determining the amount of an unused co-factor (e.g., NAD+ or NADP+) of the reaction remaining in the reaction mixture after a certain period of time. The quantity of an unused co-factor may be detected, for example, by spectrophotometric methods and/or other methods known in the art or described herein.

As used herein, the terms “anneal” and “hybridize” are used interchangeably and refer to the formation of a stable duplex of nucleic acids by way of hybridization mediated by inter-strand hydrogen bonding, for example, according to Watson-Crick base pairing. The nucleic acids of the duplex may be, for example, at least 50% complementary to one another (e.g., about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 99.9%, or 100% complementary to one another). The “stable duplex” formed upon the annealing of one nucleic acid to another is a duplex structure that is not denatured by a stringent wash. Exemplary stringent wash conditions are known in the art and include temperatures of about 5° C. less than the melting temperature of an individual strand of the duplex and low concentrations of monovalent salts, such as monovalent salt concentrations (e.g., NaCl concentrations) of less than 0.2 M (e.g., 0.2 M, 0.19 M, 0.18 M, 0.17 M, 0.16 M, 0.15 M, 0.14 M, 0.13 M, 0.12 M, 0.11 M, 0.1 M, 0.09 M, 0.08 M, 0.07 M, 0.06 M, 0.05 M, 0.04 M, 0.03 M, 0.02 M, 0.01 M, or less).

As used herein, the term “capable of producing” refers to a host cell that is genetically modified to express the enzyme(s) necessary for the production of a given compound in accordance with a biochemical pathway that produces the compound. For example, a host cell (e.g., a yeast cell) that is “capable of producing” a human milk oligosaccharide (HMO) is one that expresses the enzymes necessary for production of the HMO according to the biosynthetic pathway for the HMO of interest.

As used herein, a host cell that is “deficient” in a level of a saccharide (e.g., a HMO described herein) or a sugar-alditol (e.g., difucosyllactose (DFL)) is one that is modified so as to produce a reduced quantity and/or concentration of the saccharide or sugar-alditol relative to a wild-type cell of the same species lacking the modification of the deficient cell.

As used herein in the context of a gene or expression thereof, the term “disrupt” means to prevent the formation of a functional gene product. A gene product is functional if it fulfills its normal (wild-type) function(s). Disruption of the gene prevents expression of a functional RNA transcript or protein encoded by the gene. Disruption of the gene may be accomplished by, for example, an insertion, deletion, or substitution of one or more bases in a nucleic acid sequence of the gene or a corresponding transcription regulatory element that is operably linked to the gene, such as a promoter, enhancer, or operator that regulates expression of the gene in vivo. The disrupted gene may be disrupted by, e.g., removal of at least a portion of the gene from a genome of the animal, alteration of the gene to prevent expression of a functional factor encoded by the gene, an interfering RNA, or expression of a dominant negative factor by an exogenous gene.

As used herein, the term “endogenous” describes a molecule (e.g., a polypeptide, nucleic acid, or cofactor) that is found naturally in a particular organism (e.g., a human) or in a particular location within an organism (e.g., an organ, a tissue, or a cell, such as a human cell).

As used herein, the term “exogenous” describes a molecule (e.g., a polypeptide, nucleic acid, or cofactor) that is not found naturally in a particular organism (e.g., a human) or in a particular location within an organism (e.g., an organ, a tissue, or a cell, such as a human cell). Exogenous materials include those that are provided from an external source to an organism or to cultured matter extracted there from.

As used herein in the context of a gene, the term “express” refers to any one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein. Expression of a gene of interest in a cell, tissue sample, or subject can manifest, for example, as: an increase in the quantity or concentration of mRNA encoding a corresponding protein (as assessed, e.g., using RNA detection procedures described herein or known in the art, such as quantitative polymerase chain reaction (qPCR) and RNA seq techniques), an increase in the quantity or concentration of a corresponding protein (as assessed, e.g., using protein detection methods described herein or known in the art, such as enzyme-linked immunosorbent assays (ELISA), among others), and/or an increase in the activity of a corresponding protein (e.g., in the case of an enzyme, as assessed using an enzymatic activity assay described herein or known in the art).

The term “expression cassette” or “expression construct” refers to a nucleic acid construct that, when introduced into a host cell, results in transcription and/or translation of an RNA or polypeptide, respectively. In the case of expression of transgenes, one of skill will recognize that the inserted polynucleotide sequence need not be identical but may be only substantially identical to a sequence of the gene from which it was derived. As explained herein, these substantially identical variants are specifically covered by reference to a specific nucleic acid sequence. One example of an expression cassette is a polynucleotide construct that includes a polynucleotide sequence encoding a polypeptide for use in the invention operably linked to a promoter, e.g., its native promoter, where the expression cassette is introduced into a heterologous microorganism. In some embodiments, an expression cassette includes a polynucleotide sequence encoding a polypeptide of the invention where the polynucleotide that is targeted to a position in the genome of a microorganism such that expression of the polynucleotide sequence is driven by a promoter that is present in the microorganism.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refers to a set of at least two different coding sequences, where the coding sequences encode enzymes that catalyze different parts of a synthetic pathway to form a desired product (e.g., a HMO). In a genetic pathway, a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

The term “host cell” as used in the context of this disclosure refers to a microorganism, such as yeast, and includes an individual cell or cell culture including a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

The terms “human milk oligosaccharide” and “HMO” are used interchangeably herein to refer to a group of nearly 200 identified sugar molecules that are found as the third most abundant component in human breast milk. HMOs in human breast milk are a complex mixture of free, indigestible carbohydrates with many different biological roles, including promoting the development of a functional infant immune system. HMOs include, without limitation, 2′-fucosyllactose (2′-FL), lacto-N-neotetraose (LNnT), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, and fucosyl-disialyllacto-N-hexaose (FDS-LNH II), among others.

The terms “variant LgtA” and “variant β-1,3-N-acetylglucosaminyltransferase” refer to a polypeptide having at least one (e.g., 1, 2, 3, 4, 5, 6, 7, 8, 9, 10, or more) amino acids substitutions or deletions relative to a wild-type LgtA polypeptide (e.g., a wild-type LgtA polypeptide from N. meningitidis, the amino acid sequence of which is set forth in SEQ ID NO: 104). The LgtA polypeptide may be modified (e.g., by way of one or more of the amino acid substitutions or deletions described herein) to enhance its specificity for binding to, and catalyzing the glycosidation of, the enzyme's intended substrate in the biosynthetic pathway of a HMO relative to a longer-chain oligosaccharide.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by (the fraction X/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid.

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present disclosure will generally contain phosphodiester bonds, although in some cases, nucleic acid analogs may be used that may have alternate backbones, including, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using a sequence comparison algorithm or by manual alignment and visual inspection as described above. Optionally, the identity exists over a region that is at least about 50 nucleotides (or 20 amino acids) in length, or more preferably over a region that is 100 to 500 or 1000 or more nucleotides (or 50, 100, or 200 or more amino acids) in length.

Nucleic acid or protein sequences that are substantially identical to a reference sequence include “conservatively modified variants.” With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Examples of amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R) and His (Histidine or H); an “aromatic or cyclic group” including Pro (Proline or P), Phe (Phenylalanine or F), Tyr (Tyrosine or Y) and Trp (Tryptophan or W); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T) and Cys (Cysteine or C). Within each group, subgroups can also be identified. For example, at pH 7, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His and Trp; and the “phenyl sub-group” comprising Phe and Tyr. In another further example, the aliphatic group can be sub-divided into sub-groups including: the “large aliphatic non-polar sub-group” comprising Val, Leu, and Ile; the “aliphatic slightly-polar sub-group” comprising Met, Ser, Thr and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free-OH can be maintained; and Gln for Asn or vice versa, such that a free —NH₂ can be maintained. The following six groups each contain amino acids that further provide illustrative conservative substitutions for one another. 1) Ala, Ser, Thr; 2) Asp, Glu; 3) Asn, Gln; 4) Arg, Lys; 5) Ile, Leu, Met, Val; and 6) Phe, Try, and Trp (see, e.g., Creighton, Proteins: Structures and Molecular Principles. 1984, New York: W.H. Freeman).

Accordingly, the terms “conservative mutation,” “conservative substitution,” and “conservative amino acid substitution” refer to a substitution of one or more amino acids for one or more different amino acids that exhibit similar physicochemical properties, such as polarity, electrostatic charge, and steric volume. These properties are summarized for each of the twenty naturally-occurring amino acids in Table 1, below.

TABLE 1
Representative physicochemical properties of naturally-occurring amino
acids

| | 3 | 1 | Side- | Electrostatic | |
|---|---|---|---|---|---|
| | Letter | Letter | chain | character | Steric |
| Amino Acid | Code | Code | Polarity | at pH = 7.4 | Volume^{†} |
| Alanine | Ala | A | nonpolar | neutral | small |
| Arginine | Arg | R | polar | cationic | large |
| Asparagine | Asn | N | polar | neutral | intermediate |
| Aspartic acid | Asp | D | polar | anionic | intermediate |
| Cysteine | Cys | C | nonpolar | neutral | intermediate |
| Glutamic acid | Glu | E | polar | anionic | intermediate |
| Glutamine | Gln | Q | polar | neutral | intermediate |
| Glycine | Gly | G | nonpolar | neutral | small |
| Histidine | His | H | polar | Both neutral and | large |
| | | | | cationic forms | |
| Isoleucine | Ile | | nonpolar | neutral | large |
| Leucine | Leu | L | nonpolar | neutral | large |
| Lysine | Lys | K | polar | cationic | large |
| Methionine | Met | M | nonpolar | neutral | large |
| Phenylalanine | Phe | F | nonpolar | neutral | large |
| Proline | Pro | P | non- | neutral | intermediate |
| | | | polar | | |
| Serine | Ser | S | polar | neutral | small |
| Threonine | Thr | T | polar | neutral | intermediate |
| Tryptophan | Trp | W | nonpolar | neutral | bulky |
| Tyrosine | Tyr | Y | polar | neutral | large |
| Valine | Val | V | nonpolar | neutral | intermediate |

^{†}based on volume in A³: 50-100 is small, 100-150 is intermediate, 150-200 is large, and >200 is bulky

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of compound produced per volume per time.

As used herein, the term “overexpression” refers to a process of genetically modifying a host cell to express a polypeptide or RNA molecule in an amount that exceeds the amount of the polypeptide or RNA that would be observed in a host cell of the same species but that has not been subject to the genetic modification. Exemplary methods of overexpressing a polypeptide or RNA molecule of the disclosure include expressing the polypeptide or RNA molecule in a host cell under the control of a highly active transcription regulatory element, such as a promoter or enhancer that fosters expression of the polypeptide or RNA at levels that exceed wild-type expression levels observed in an unmodified host cell of the same species.

As used herein, the term “promoter” refers to a synthetic or naturally-derived nucleic acid that is capable of activating, increasing, or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

The term “reducing sugar” refers to a saccharide that contains a free aldehyde functional group or that can tautomerize in solution (e.g., in aqueous solution) to form an aldehyde group. Some disaccharides, oligosaccharides, polysaccharides, and all monosaccharides are reducing sugars. The monosaccharides can categorized into two groups: (1) aldoses that contain a free aldehyde group and (2) ketoses containing a ketone group. Ketoses must tautomerize to aldoses before acting as a reducing agent. Reducing sugars can be readily identified by way of a Tollens' test. A Tollens' test may be used to differentiate reducing sugars from non-reducing sugars. In a Tollens' test, a Tollens' reagent including silver ions and aqueous ammonia is added to a solution including the sugar of interest. The sugar may be identified as a reducing sugar if silver metal precipitates upon addition of the Tollens' reagent to the sugar of interest. For example, in some embodiments, the reducing sugar is lactose. In some embodiments, the reduced form of the reducing sugar (e.g., lactose) is lactitol. In some embodiments, the reducing sugar is 2′-FL. In some embodiments, the reducing sugar is 6′-SL. In some embodiments, the reducing sugar is LNnT. In some embodiments, the reduced form of the reducing sugar (e.g., LNnT) is LNnT-alditol. In some embodiments, the reduced form of the reducing sugar is 2′-fucosyllactitol.

As used herein, the term “heterologous” refers to what is not normally found in nature. The term “heterologous nucleic acid” refers to a nucleic acid not normally found in a given cell in nature. A heterologous nucleic acid can be: (a) foreign to its host cell, i.e., exogenous to the host cell such that a host cell does not naturally contain the nucleic acid; (b) naturally found in the host cell, i.e., endogenous or native to the host cell, but present at an unnatural quantity in the cell (i.e., greater or lesser quantity than naturally found in the host cell); (c) be naturally found in the host cell but positioned outside of its natural locus. A “heterologous” polypeptide refers to a polypeptide that is encoded by a “heterologous nucleic acid”. Thus, for example, a “heterologous” polypeptide may be naturally produced by a host cell but is encoded by a heterologous nucleic acid that has been introduced into the host cell by genetic engineering. For example, a “heterologous” polypeptide can include embodiments in which an endogenous polypeptide is produced by an expression construct and is overexpressed in the host cell compared to native levels of the polypeptide produced by the host cell.

As used herein, the terms “interfering ribonucleic acid” and “interfering RNA” refer to a RNA, such as a short interfering RNA (siRNA), micro RNA (miRNA), or short hairpin RNA (shRNA) that suppresses the expression of a target RNA transcript by way of (i) annealing to the target RNA transcript, thereby forming a nucleic acid duplex; (ii) promoting the nuclease-mediated degradation of the RNA transcript; and/or (iii) slowing, inhibiting, or preventing the translation of the RNA transcript, such as by sterically precluding the formation of a functional ribosome-RNA transcript complex or otherwise attenuating formation of a functional protein product from the target RNA transcript. Interfering RNAs as described herein may be provided to a patient in the form of, for example, a single- or double-stranded oligonucleotide, or in the form of a vector (e.g., a viral vector) containing a transgene encoding the interfering RNA. Exemplary interfering RNA platforms are described, for example, in Lam et al., Molecular Therapy—Nucleic Acids 4: e252 (2015); Rao et al., Advanced Drug Delivery Reviews 61:746-769 (2009); and Borel et al., Molecular Therapy 22:692-701 (2014), the disclosures of each of which are incorporated herein by reference in their entirety.

As used herein, the term “introducing” in the context of a nucleic acid or protein in a host cell refers to any process that results in the presence of a heterologous nucleic acid or polypeptide inside the host cell. For example, the term encompasses introducing a nucleic acid molecule (e.g., a plasmid or a linear nucleic acid) that encodes the nucleic acid of interest (e.g., an RNA molecule) or polypeptide of interest and results in the transcription of the RNA molecules and translation of the polypeptides. The term also encompasses integrating the nucleic acid encoding the RNA molecules or polypeptides into the genome of a progenitor cell. The nucleic acid is then passed through subsequent generations to the host cell, so that, for example, a nucleic acid encoding an RNA-guided endonuclease is “pre-integrated” into the host cell genome. In some cases, introducing refers to translocation of a nucleic acid or polypeptide from outside the host cell to inside the host cell. Various methods of introducing nucleic acids, polypeptides and other biomolecules into host cells are contemplated, including but not limited to, electroporation, contact with nanowires or nanotubes, spheroplasting, PEG 1000-mediated transformation, biolistics, lithium acetate transformation, lithium chloride transformation, and the like.

As used herein, the term “transformation” refers to a genetic alteration of a host cell resulting from the introduction of exogenous genetic material, e.g., nucleic acids, into the host cell.

As used herein, the term “mutation” refers to a change in the nucleotide sequence of a gene. Mutations in a gene may occur naturally as a result of, for example, errors in DNA replication, DNA repair, irradiation, and exposure to carcinogens or mutations may be induced as a result of administration of a transgene expressing a mutant gene. Mutations may result from a single nucleotide substitution or deletion.

As used herein, the term “antibody” (Ab) refers to an immunoglobulin molecule that specifically binds to, or is immunologically reactive with, a particular antigen, and includes polyclonal, monoclonal, genetically engineered, and otherwise modified forms of antibodies, including, but not limited to, chimeric antibodies, humanized antibodies, heteroconjugate antibodies (e.g., bi-tri- and quad-specific antibodies, diabodies, triabodies, and tetrabodies), and antigen-binding fragments of antibodies, including e.g., Fab′, F(ab′)₂, Fab, Fv, rIgG, and scFv fragments. In some embodiments, two or more portions of an immunoglobulin molecule are covalently bound to one another, e.g., via an amide bond, a thioether bond, a carbon-carbon bond, a disulfide bridge, or by a linker, such as a linker described herein or known in the art. Antibodies also include antibody-like protein scaffolds, such as the tenth fibronectin type III domain (¹⁰Fn3), which contains BC, DE, and FG structural loops similar in structure and solvent accessibility to antibody complementarity-determining regions (CDRs). The tertiary structure of the ¹⁰Fn3 domain resembles that of the variable region of the IgG heavy chain, and one of skill in the art can graft, e.g., the CDRs of a reference antibody onto the fibronectin scaffold by replacing residues of the BC, DE, and FG loops of ¹⁰Fn3 with residues from the CDR-H1, CDR-H2, or CDR-H3 regions, respectively, of the reference antibody.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be a Fab, F(ab′)₂, scFv, SMIP, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, V_{H}, C_{L}, and C_{H}1 domains; (ii) a F(ab′)2 fragment, a bivalent fragment comprising two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb including V_{H} and V_{L} domains; (vi) a dAb fragment (Ward et al., Nature 341:544-546, 1989), which consists of a V_{H} domain; (vii) a dAb which consists of a V_{H} or a V_{L} domain; (viii) an isolated CDR; and (ix) a combination of two or more isolated CDRs which may optionally be joined by a synthetic linker. Furthermore, although the two domains of the Fv fragment, V_{L} and V_{H}, are coded for by separate genes, they can be joined, using recombinant methods, by a linker that enables them to be made as a single protein chain in which the V_{L} and V_{H} regions pair to form monovalent molecules (known as single-chain Fv (scFv); see, e.g., Bird et al., Science 242:423-426, 1988, and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in some embodiments, by chemical peptide synthesis procedures known in the art.

As used herein, the term “operably linked” refers to a functional linkage between nucleic acid sequences such that the sequences encode a desired function. For example, a coding sequence for a gene of interest is in operable linkage with its promoter and/or regulatory sequences when the linked promoter and/or regulatory region functionally controls expression of the coding sequence. It also refers to the linkage between coding sequences such that they may be controlled by the same linked promoter and/or regulatory region; such linkage between coding sequences may also be referred to as being linked in frame or in the same coding frame. “Operably linked” also refers to a linkage of functional but non-coding sequences, such as an autonomous propagation sequence or origin of replication. Such sequences are in operable linkage when they are able to perform their normal function, e.g., enabling the replication, propagation, and/or segregation of a vector bearing the sequence in a host cell.

The term “yield” refers to production of a compound by a host cell, expressed as the amount of compound produced per amount of carbon source consumed by the host cell, by weight.

As used in this specification and the appended claims, the singular forms “a,” “an,” and “the” include plural reference unless the context clearly dictates otherwise.

As used herein, the term “about” is used herein to mean a value that is +10% of the recited value.

### BRIEF DESCRIPTION OF DRAWINGS

FIG. 1 is a schematic showing the 2′-fucosyllactose (2′-FL) biosynthesis pathway in S. cerevisiae. The pathway involves the activity of several enzymes, including GDP-mannose dehydratase (GMD), GDP-fucose synthase (GFS), fucosyltransferase (FT), and lactose permease (LP). Sugars are shown in rectangles, enzymes are show in ovals, and cofactors are shown unbounded.

FIG. 2A is a bar graph showing the degree of 2′-FL production resulting from the screening of 41 putative fucosyltransferases in microtiter plates.

FIG. 2B is a bar graph showing the relative amount of difucosyllactose (DFL) production resulting from the screening of 41 putative fucosyltransferases in microtiter plates.

FIG. 2C is a bar graph showing the percentage of the total fucosylated sugar produced that is 2′-FL (black) or DFL (gray) in yeast strains modified to express a series of putative fucosyltransferases.

FIG. 3 is a bar graph showing the percentage of total fucosylated sugar that is 2′-FL (black) or DFL (gray) produced in yeast strains modified to express a series of putative fucosyltransferases under microfermentor fed-batch conditions.

FIG. 4 is a bar graph showing the percentage of total fucosylated sugar that is 2′-FL (black) or DFL (gray) in broth following bench-scale fermentation of yeast strains expressing the fucosyltransferase represented by SEQ ID NO: 1 and SEQ ID NO: 4.

FIG. 5 is a graph showing the relative amount of 2′-FL production resulting from the screening of 23 putative GDP-mannose 4,6-dehydratase (GMD) enzymes in microtiter plates.

FIG. 6 is a bar graph showing the relative amount of 2′-FL production resulting from the screening of 35 putative lactose permease enzymes in microtiter plates.

FIG. 7 is a bar graph showing the relative amount of difucosyllactose (DFL) production resulting from the screening of 7 putative lactose permease enzymes in microtiter plates.

FIG. 8 is a graph showing the relative amount of 2′-FL production resulting from the screening of 4 different GDP-L-fucose synthase enzymes in microtiter plates.

FIG. 9 are graphs showing the yield of sucrose produced by and the productivity of two different yeast strains that overexpress PSA1 and SEC53 in comparison to the two yeast strains that do not overexpress PSA1 and SEC53 over time.

FIG. 10 are graphs showing the yield of sucrose produced by and the productivity of parent and child yeast strains that overexpress IMD3 and GUA1 in comparison to the parent and child yeast strains that do not overexpress IMD3 and GUA1 over time.

FIG. 11 are graphs showing the yield of sucrose produced by and the productivity of yeast strains that overexpress HEM12 alone or HEM12 and SAK1 together over time.

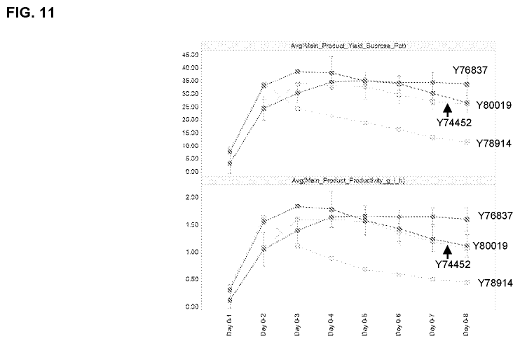

FIG. 12 are graphs showing the yield of sucrose produced by and the productivity of yeast strains that overexpress HEM12 alone or HEM12 and SAK1 together over time.

FIG. 13 are graphs showing that deletion of the maltose regulon in strains showed better 2′-FL yield and productivity in comparison to comparable strains without these deletions.

FIG. 14 is a series of graphs showing strain screening using AMBR fermentation to explore multiple lactose-challenge RNA-seq analysis for strains that overexpress DAN1, SIP18, or HSP26.

FIG. 15 is a series of graphs showing the comparison of an ACS1 overexpression strain to its parental strain in high lactose feed conditions.

FIG. 16 is a series of graphs showing the amount of dissolved oxygen, the feed rate, the oxygen uptake rate, the temperature, and the total cell density over time for strains having that overexpressed NPY1, RFT1, FZO1, or OLE1.

FIG. 17 is a series of graphs showing the yield, productivity, cell density, and amount of lactose added for strains that overexpressed NPY1, RFT1, FZO1, or OLE1 over time.

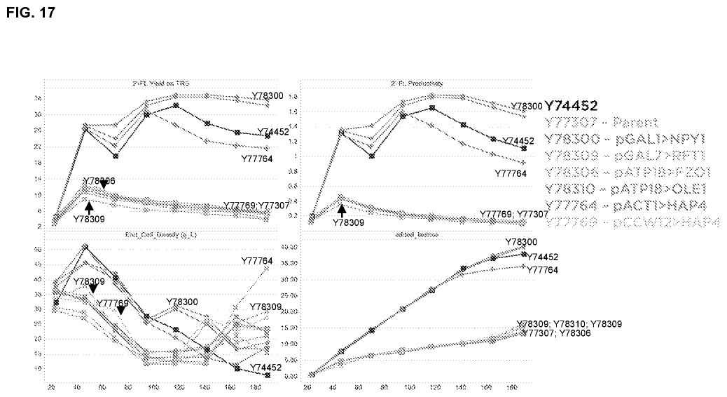

FIG. 18 is a graph showing the concentration of 6′-SL produced by a knockout strain wherein the maltose regulon is deleted.

FIG. 19 is a series of graphs showing the yield of 6′-SL and the productivity of strains either having or lacking the maltose regulon.

### DETAILED DESCRIPTION

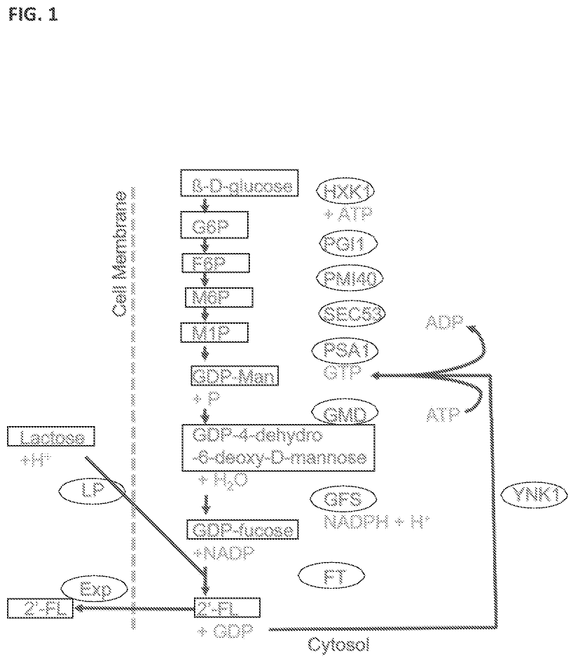

The present disclosure features host cells capable of producing one or more human milk oligosaccharides (HMOs), as well as methods of using such host cells to produce a HMO in high overall yield while simultaneously suppressing the formation of undesirable impurities. The host cells described herein may, for example, encode one or more heterologous nucleic acids encoding one or more enzymes of the HMO biosynthetic pathway as described in FIG. 1. In some embodiments, the host cell may encode a fucosyltransferase, a GDP-mannose dehydratase, a lactose permease, and/or a fucose synthase, and the host cell may be capable of producing the HMO 2′-fucosyllactose (2′-FL), among other HMOs described herein.

It has presently been discovered that host cells expressing one or more of the HMO biosynthetic enzymes described herein are capable of producing a desired HMO with heightened purity and overall yield relative to host cells that do not express one or more of the HMO biosynthetic enzymes of the disclosure. The following sections provide a detailed description of the host cells that may be used to produce a HMO with elevated overall yield and purity, as well as exemplary techniques for preparing such modified host cells.

### Enzymes of the Biosynthetic Pathway of a Target HMO

The host cells described herein may be modified so as to express one or more enzymes of the biosynthetic pathway of a target HMO. In some embodiments, for example, host cells of the disclosure (e.g., yeast cells) may naturally express some of the enzymes of the biosynthetic pathway for a given HMO. Such host cells may be modified to express the remaining or heterologous enzymes of the biosynthetic pathway. In some embodiments, for instance, a host cell (e.g., a yeast cell) may naturally express many of the enzymes of the biosynthetic pathway of a desired HMO (e.g., 2′-FL, LNnT, 6′-SL), and the host cells may be modified so as to express the remaining enzymes of the biosynthetic pathway for the desired HMO by providing the cells with one or more heterologous nucleic acid molecules that, together, encode the remaining enzymes of the biosynthetic pathway.

In some embodiments, host cells of the disclosure are modified so as to express one or more enzymes of the biosynthetic pathway of a fucose-containing HMO, such as 2′-FL or 6′-SL. The one or more enzymes may include, for example, a fucosyltransferase, GDP-mannose 4,6-dehydratase (GMD), lactose permease, and/or a fucose synthase. In some embodiments, for example, a host cell of the disclosure is modified to express: a fucosyltransferase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 1-41; and/or a GMD having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 42-64; and/or a lactose permease having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 65-99; and/or a fucose synthase having an amino acid sequence that is at least 85% identical (e.g., at least 85%, 86%, 87%, 88%, 89%, 90%, 91%, 92%, 93%, 94%, 95%, 96%, 97%, 98%, 99%, or 100% identical) to the amino acid sequence of any one of SEQ ID NOS: 100-103.

Additionally, host cells of the disclosure may be modified to express other enzymes of a biosynthetic pathway of a desired HMO. For example, a host cell of the disclosure may be modified to express one or more of a β-1,3-N-acetylglucosaminyltransferase (LgtA), a β-1,4-galactosyltransferase (LgtB), and a UDP-N-acetylglucosamine diphosphorylase. Such enzymes may be expressed, for example, in host cells so as to produce LNnT.

In some embodiments, host cells of the disclosure are provided with heterologous nucleic acid molecules that encode one or more enzymes of a pathway for synthesizing lacto-N-tetraose, including a LgtA, a β-1,3-galactosyltransferase, and a UDP-N-acetylglucosamine diphosphorylase.

In some embodiments, host cells of the disclosure are provided with heterologous nucleic acid molecules that encode one or more enzymes of a pathway for synthesizing 3′-sialyllactose, including a CMP-Neu5Ac synthetase, a sialic acid synthase, a UDP-N-acetylglucosamine 2-epimerase, a UDP-N-acetylglucosamine diphosphorylase, and a CMP-N-acetylneuraminate-β-galactosamide-α-2,3-sialyltransferase.

In some embodiments, host cells of the disclosure are provided with heterologous nucleic acid molecules that encode one or more enzymes of a pathway for synthesizing 6′-sialyllactose, including a CMP-Neu5Ac synthetase, a sialic acid synthase, a UDP-N-acetylglucosamine 2-epimerase, a UDP-N-acetylglucosamine diphosphorylase, and a β-galactoside-α-2,6-sialyltransferase.

In some embodiments, host cells of the disclosure are provided with heterologous nucleic acid molecules that encode one or more enzymes of a pathway for synthesizing difucosyllactose, including a GDP-mannose 4,6-dehydratase, a GDP-L-fucose synthase, a fucosyltransferase, and an α-1,3-fucosyltransferase.

Exemplary heterologous enzymes useful in conjunction with the compositions and methods of the disclosure are described in the sections that follow.

### Fucosyltransferase

In some embodiments, the host cells of the disclosure express a fucosyltransferase polypeptide. In some embodiments, the fucosyltransferase has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 1-41. In some embodiments, the fucosyltransferase has an amino acid sequence that is at least 90% identical (e.g., at least 91%, 92%, 93%, 94%, 95%, 96%, 97%, 98%, or 99% identical) to the amino acid sequence of any one of SEQ ID NOS: 1-41. In some embodiments, the fucosyltransferase has an amino acid sequence that is at least 95% identical (e.g., at least 96%, 97%, 98%, or 99% identical) to the amino acid sequence of any one of SEQ ID NOS: 1-41. In some embodiments, the fucosyltransferase has the amino acid sequence of any one of SEQ ID NOS: 1-41.

In some embodiments, the fucosyltransferase has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 1-3 and 6-41. In some embodiments, the fucosyltransferase has an amino acid sequence that is at least 90% identical (e.g., at least 91%, 92%, 93%, 94%, 95%, 96%, 97%, 98%, or 99% identical) to the amino acid sequence of any one of SEQ ID NOS: 1-3 and 6-41. In some embodiments, the fucosyltransferase has an amino acid sequence that is at least 95% identical (e.g., at least 96%, 97%, 98%, or 99% identical) to the amino acid sequence of any one of SEQ ID NOS: 1-3 and 6-41. In some embodiments, the fucosyltransferase has the amino acid sequence of any one of SEQ ID NOS: 1-3 and 6-41.

In some embodiments, the fucosyltransferase has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 1-3. In some embodiments, the fucosyltransferase has an amino acid sequence that is at least 90% identical (e.g., at least 91%, 92%, 93%, 94%, 95%, 96%, 97%, 98%, or 99% identical) to the amino acid sequence of any one of SEQ ID NOS: 1-3. In some embodiments, the fucosyltransferase has an amino acid sequence that is at least 95% identical (e.g., at least 96%, 97%, 98%, or 99% identical) to the amino acid sequence of any one of SEQ ID NOS: 1-3. In some embodiments, the fucosyltransferase has the amino acid sequence of any one of SEQ ID NOS: 1-3.

### GDP-Mannose 4,6-Dehydratase

In some embodiments, the host cells of the disclosure express a GDP-mannose 4,6-dehydratase (GMD). In some embodiments, the GMD has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 42-64. In some embodiments, the GMD has an amino acid sequence that is at least 90% identical (e.g., at least 91%, 92%, 93%, 94%, 95%, 96%, 97%, 98%, or 99% identical) to the amino acid sequence of any one of SEQ ID NOS: 42-64. In some embodiments, the GMD has an amino acid sequence that is at least 95% identical (e.g., at least 96%, 97%, 98%, or 99% identical) to the amino acid sequence of any one of SEQ ID NOS: 42-64. In some embodiments, the GMD has the amino acid sequence of any one of SEQ ID NOS: 42-64.

In some embodiments, the GMD has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 42-44. In some embodiments, the GMD has an amino acid sequence that is at least 90% identical (e.g., at least 91%, 92%, 93%, 94%, 95%, 96%, 97%, 98%, or 99% identical) to the amino acid sequence of any one of SEQ ID NOS: 42-44. In some embodiments, the GMD has an amino acid sequence that is at least 95% identical (e.g., at least 96%, 97%, 98%, or 99% identical) to the amino acid sequence of any one of SEQ ID NOS: 42-44. In some embodiments, the GMD has the amino acid sequence of any one of SEQ ID NOS: 42-44.

### Lactose Importer

In some embodiments, the host cells of the disclosure express a protein that transports lactose into the host cell, such as a lactose permease. In some embodiments, the lactose permease has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 65-99. In some embodiments, the lactose permease has an amino acid sequence that is at least 90% identical (e.g., at least 91%, 92%, 93%, 94%, 95%, 96%, 97%, 98%, or 99% identical) to the amino acid sequence of any one of SEQ ID NOS: 65-99. In some embodiments, the lactose permease has an amino acid sequence that is at least 95% identical (e.g., at least 96%, 97%, 98%, or 99% identical) to the amino acid sequence of any one of SEQ ID NOS: 65-99. In some embodiments, the lactose permease has the amino acid sequence of any one of SEQ ID NOS: 65-99.

### Fucose Synthase

In some embodiments, the host cells of the disclosure express a fucose synthase. In some embodiments, the fucose synthase has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 100-103. In some embodiments, the fucose synthase has an amino acid sequence that is at least 90% identical (e.g., at least 91%, 92%, 93%, 94%, 95%, 96%, 97%, 98%, or 99% identical) to the amino acid sequence of any one of SEQ ID NOS: 100-103. In some embodiments, the fucose synthase has an amino acid sequence that is at least 95% identical (e.g., at least 96%, 97%, 98%, or 99% identical) to the amino acid sequence of any one of SEQ ID NOS: 100-103. In some embodiments, the fucose synthase has the amino acid sequence of any one of SEQ ID NOS: 100-103.

### β-1,3-N-acetylglucosaminyltransferase Polypeptides

The LgtA polypeptides of the disclosure can be used to produce one or more of a variety of HMOs, including, without limitation, LNnT, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, LST a, LST b, LST c, DS-LNT, F-LST a, F-LST b, FS-LNH, FS-LNnH I, and FDS-LNH II.

In some embodiments, a LgtA polypeptide of the disclosure contains one or more amino acid substitutions relative to the wild-type LgtA amino acid sequence set forth in SEQ ID NO: 104. The amino acid substitution may occur, for example, at a residue selected from P89, G179, N180, I182, H183, N185, T186, M187, W206, A207, Q211, W213, L229, V230, R233, H235, S240, K242, Y243, Q247, I250, I254, Q255, A258, L288, and E294 of SEQ ID NO: 104.

In some embodiments, the LgtA polypeptide includes an amino acid substitution at residue G179 of SEQ ID NO: 104. For example, the amino acid substitution at residue G179 of SEQ ID NO: 104 may substitute G179 with an amino acid including a cationic side chain at physiological pH. In some embodiments, the amino acid substitution at residue G179 of SEQ ID NO: 104 is a G179R substitution.

In some embodiments, the LgtA polypeptide includes an amino acid substitution at residue P89 of SEQ ID NO: 104. For example, the amino acid substitution at residue P89 of SEQ ID NO: 104 may substitute P89 with an amino acid including a polar, uncharged chain at physiological pH. In some embodiments, the amino acid substitution at residue P89 of SEQ ID NO: 104 is a P89T substitution.

In some embodiments, the LgtA polypeptide includes an amino acid substitution at residue N180 of SEQ ID NO: 104. For example, the amino acid substitution at residue N180 of SEQ ID NO: 104 may substitute N180 with an amino acid including an anionic side chain at physiological pH. In some embodiments, the amino acid substitution at residue N180 of SEQ ID NO: 104 is an N180D substitution. In some embodiments, the amino acid substitution at residue N180 of SEQ ID NO: 104 substitutes N180 with an amino acid including a hydrophobic, uncharged side chain at physiological pH. For example, the amino acid substitution at residue N180 of SEQ ID NO: 104 may be an N180A substitution.

In some embodiments, the LgtA polypeptide includes an amino acid substitution at residue I182 of SEQ ID NO: 104. In some embodiments, the amino acid substitution at residue I182 of SEQ ID NO: 104 substitutes I182 with an amino acid including a hydrophobic, uncharged side chain at physiological pH. For example, the amino acid substitution at residue I182 of SEQ ID NO: 104 may be an I182Y substitution.

In some embodiments, the LgtA polypeptide includes an amino acid substitution at residue H183 of SEQ ID NO: 104. For example, the amino acid substitution at residue H183 of SEQ ID NO: 104 may be an H183P substitution. In some embodiments, the amino acid substitution at residue H183 of SEQ ID NO: 104 substitutes H183 with an amino acid including a polar, uncharged side chain at physiological pH. For example, the amino acid substitution at residue H183 of SEQ ID NO: 104 may be an H183S substitution.

In some embodiments, the LgtA polypeptide includes an amino acid substitution at residue N185 of SEQ ID NO: 104. For example, the amino acid substitution at residue N185 of SEQ ID NO: 104 may be an N185G substitution.

In some embodiments, the LgtA polypeptide includes an amino acid substitution at residue T186 of SEQ ID NO: 104. In some embodiments, the amino acid substitution at residue T186 of SEQ ID NO: 104 substitutes T186 with an amino acid including an anionic side chain at physiological pH. For example, the amino acid substitution at residue T186 of SEQ ID NO: 104 may be a T186D substitution. In another example, the amino acid substitution at residue T186 of SEQ ID NO: 104 may be a T186G substitution.

In some embodiments, the LgtA polypeptide includes an amino acid substitution at residue M187 of SEQ ID NO: 104. For example, the amino acid substitution at residue M187 of SEQ ID NO: 104 may be an M187P substitution.

In some embodiments, the LgtA polypeptide includes an amino acid substitution at residue W206 of SEQ ID NO: 104. The amino acid substitution at residue W206 of SEQ ID NO: 104 may substitute W206 with an amino acid including a polar, uncharged side chain at physiological pH. For example, the amino acid substitution at residue W206 of SEQ ID NO: 104 may be a W206N substitution.

In some embodiments, the LgtA polypeptide includes an amino acid substitution at residue A207 of SEQ ID NO: 104. In some embodiments, the amino acid substitution at residue A207 of SEQ ID NO: 104 substitutes A207 with an amino acid including a hydrophobic, uncharged side chain at physiological pH. For example, the amino acid substitution at residue A207 of SEQ ID NO: 104 may be an A207V substitution.

In some embodiments, the LgtA polypeptide includes an amino acid substitution at residue Q211 of SEQ ID NO: 104. The amino acid substitution at residue Q211 of SEQ ID NO: 104 may substitute Q211 with an amino acid including a hydrophobic, uncharged side chain at physiological pH. For example, the amino acid substitution at residue Q211 of SEQ ID NO: 104 may be a Q211V substitution, a Q2111 substitution, or a Q211L substitution. In some embodiments, the amino acid substitution at residue Q211 of SEQ ID NO: 104 is a Q211C substitution.

In some embodiments, the LgtA polypeptide includes an amino acid substitution at residue W213 of SEQ ID NO: 104. In some embodiments, the amino acid substitution at residue W213 of SEQ ID NO: 104 substitutes W213 with an amino acid including a polar, uncharged side chain at physiological pH. For example, the amino acid substitution at residue W213 of SEQ ID NO: 104 is a W213S substitution or a W213N substitution.

In some embodiments, the LgtA polypeptide includes an amino acid substitution at residue L229 of SEQ ID NO: 104. In some embodiments, the amino acid substitution at residue L229 of SEQ ID NO: 104 substitutes L229 with an amino acid including a hydrophobic, uncharged side chain at physiological pH. For example, the amino acid substitution at residue L229 of SEQ ID NO: 104 may be an L229A substitution. In some embodiments, the amino acid substitution at residue L229 of SEQ ID NO: 104 is an L229P substitution.

In some embodiments, the LgtA polypeptide includes an amino acid substitution at residue V230 of SEQ ID NO: 104. The amino acid substitution at residue V230 of SEQ ID NO: 104 may substitute V230 with an amino acid including an anionic side chain at physiological pH. For example, the amino acid substitution at residue V230 of SEQ ID NO: 104 is a V230D substitution.

In some embodiments, the LgtA polypeptide includes an amino acid substitution at residue R233 of SEQ ID NO: 104. The amino acid substitution at residue R233 of SEQ ID NO: 104 may substitute R233 with an amino acid including a hydrophobic, uncharged side chain at physiological pH. For example, the amino acid substitution at residue R233 of SEQ ID NO: 104 may be an R2331 substitution.

In some embodiments, the LgtA polypeptide includes an amino acid substitution at residue H235 of SEQ ID NO: 104. In some embodiments, the amino acid substitution at residue H235 of SEQ ID NO: 104 substitutes H235 with an amino acid including a cationic side chain at physiological pH. For example, the amino acid substitution at residue H235 of SEQ ID NO: 104 may be an H235R substitution.

In some embodiments, the LgtA polypeptide includes an amino acid substitution at residue S240 of SEQ ID NO: 104. The amino acid substitution at residue S240 of SEQ ID NO: 104 may substitute S240 with an amino acid including a polar, uncharged side chain at physiological pH. For example, the amino acid substitution at residue S240 of SEQ ID NO: 104 may be an S240N substitution. Furthermore, the amino acid substitution at residue S240 of SEQ ID NO: 104 may be an S240Y substitution.

In some embodiments, the LgtA polypeptide includes an amino acid substitution at residue K242 of SEQ ID NO: 104. In some embodiments, the amino acid substitution at residue K242 of SEQ ID NO: 104 substitutes K242 with an amino acid including an anionic side chain at physiological pH. For example, the amino acid substitution at residue K242 of SEQ ID NO: 104 may be a K242D substitution.

In some embodiments, the LgtA polypeptide includes an amino acid substitution at residue Y243 of SEQ ID NO: 104. The amino acid substitution at residue Y243 of SEQ ID NO: 104 may substitute Y243 with an amino acid including a polar, uncharged side chain at physiological pH. For example, the amino acid substitution at residue Y243 of SEQ ID NO: 104 may be a Y243S substitution. Furthermore, the amino acid substitution at residue Y243 of SEQ ID NO: 104 may be a Y243A substitution or a Y243L substitution. In some embodiments, the amino acid substitution at residue Y243 of SEQ ID NO: 104 substitutes Y243 with an amino acid including a cationic side chain at physiological pH. For example, the amino acid substitution at residue Y243 of SEQ ID NO: 104 may be a Y243R substitution.

In some embodiments, the LgtA polypeptide includes an amino acid substitution at residue Q247 of SEQ ID NO: 104. For example, the amino acid substitution at residue Q247 of SEQ ID NO: 104 may be a Q247C substitution.

In some embodiments, the LgtA polypeptide includes an amino acid substitution at residue L288 of SEQ ID NO: 104. In some embodiments, the amino acid substitution at residue L288 of SEQ ID NO: 104 substitutes L288 with an amino acid including a polar, uncharged side chain at physiological pH. For example, the amino acid substitution at residue L288 of SEQ ID NO: 104 may be a L288S substitution.

In some embodiments, the LgtA polypeptide includes an amino acid substitution at residue 1250 of SEQ ID NO: 104. In some embodiments, the amino acid substitution at residue 1250 of SEQ ID NO: 104 substitutes 1250 with an amino acid including a hydrophobic, uncharged side chain at physiological pH. For example, the amino acid substitution at residue 1250 of SEQ ID NO: 104 may be an I250F substitution.

In some embodiments, the LgtA polypeptide includes an amino acid substitution at residue I254 of SEQ ID NO: 104. In some embodiments, the amino acid substitution at residue I254 of SEQ ID NO: 104 substitutes I254 with an amino acid including a hydrophobic, uncharged side chain at physiological pH. For example, the amino acid substitution at residue I254 of SEQ ID NO: 104 may be an I254A substitution.

In some embodiments, the LgtA polypeptide includes an amino acid substitution at residue Q255 of SEQ ID NO: 104. The amino acid substitution at residue Q255 of SEQ ID NO: 104 may substitute Q255 with an amino acid including an anionic side chain at physiological pH. For example, the amino acid substitution at residue Q255 of SEQ ID NO: 104 may be a Q255D substitution.

In some embodiments, the LgtA polypeptide includes an amino acid substitution at residue A258 of SEQ ID NO: 104. In some embodiments, the amino acid substitution at residue A258 of SEQ ID NO: 104 substitutes A258 with an amino acid including an anionic side chain at physiological pH. For example, in some embodiments, the amino acid substitution at residue A258 of SEQ ID NO: 104 is an A258D substitution. Furthermore, in some embodiments, the amino acid substitution at residue A258 of SEQ ID NO: 104 is an A258R substitution.

In some embodiments, the LgtA polypeptide includes an amino acid substitution at residue E294 of SEQ ID NO: 104. In some embodiments, the amino acid substitution at residue E294 of SEQ ID NO: 104 substitutes E294 with an amino acid including a polar, uncharged side chain at physiological pH. In some embodiments, the amino acid substitution at residue E294 of SEQ ID NO: 104 is an E294N substitution.

In some embodiments, the one or more amino acid substitutions include a deletion of residues 301-348 of SEQ ID NO: 104.

Illustrative variant LgtA polypeptide sequences that may be used in conjunction with the compositions and methods described herein include, without limitation, SEQ ID NO: 105-120, as well as functional variants thereof.

In some embodiments, the LgtA polypeptide has an amino acid sequence that is from about 85% to about 99.7% identical (e.g., about 86%, 87%, 88%, 89%, 90%, 91%, 92%, 93%, 94%, 95%, 96%, 97%, 98%, 99%, or 99.5% identical) to the amino acid sequence of SEQ ID NO: 104. In some embodiments, the polypeptide has an amino acid sequence that is from about 90% to about 99.7% identical (e.g., about 91%, 92%, 93%, 94%, 95%, 96%, 97%, 98%, 99%, 99.5% identical) to the amino acid sequence of SEQ ID NO: 104. In some embodiments, the polypeptide has an amino acid sequence that is from about 95% to about 99.7% identical (e.g., about 96%, 97%, 98%, 99%, or 99.5% identical) to the amino acid sequence of SEQ ID NO: 104.

In some embodiments, the LgtA polypeptide has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of any one of SEQ ID NOS: 105-120. In some embodiments, the LgtA polypeptide has an amino acid sequence that is at least 90% identical (e.g., at least 91%, 92%, 93%, 94%, 95%, 96%, 97%, 98%, or 99% identical) to the amino acid sequence of any one of SEQ ID NOS: 105-120. In some embodiments, the LgtA polypeptide has an amino acid sequence that is at least 95% identical (e.g., at least 95%, 96%, 97%, 98%, or 99% identical) to the amino acid sequence of any one of SEQ ID NOS: 105-120. In some embodiments, the LgtA polypeptide has the amino acid sequence of any one of SEQ ID NOS: 105-120.

### β-1,4-galactosyltransferase Polypeptides

In some embodiments, the host cells of the disclosure express a LgtB polypeptide. In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 121. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 121. In some embodiments, the LgtB has an amino acid sequence that is at least 95% identical (e.g., at least 96%, 97%, 98%, or 99% identical) to the amino acid sequence of SEQ ID NO: 121. In some embodiments, the LgtB has the amino acid sequence of SEQ ID NO: 121.

In some embodiments, the LgtB has an amino acid sequence that is at least 85% identical (e.g., at least 86%, 87%, 88%, 89%, 90%, 91%, 92%, 93%, 94%, 95%, 96%, 97%, 98%, or 99% identical) to the amino acid sequence of SEQ ID NO: 122. In some embodiments, the LgtB has an amino acid sequence that is at least 90% identical (e.g., at least 91%, 92%, 93%, 94%, 95%, 96%, 97%, 98%, or 99% identical) to the amino acid sequence of SEQ ID NO: 122. In some embodiments, the LgtB has an amino acid sequence that is at least 95% identical (e.g., at least 96%, 97%, 98%, or 99% identical) to the amino acid sequence of SEQ ID NO: 122. In some embodiments, the LgtB has the amino acid sequence of SEQ ID NO: 122.

### Host Cells Genetically Modified to Produce HMOs

Provided herein are genetically modified host cells (e.g., yeast cells) capable of producing one or more HMOs, such as one or more of LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II, among others. In some embodiments, the genetically modified host cells are capable of producing 2′-FL. In some embodiments, the genetically modified host cells are capable of producing 6′-SL. The host cells (e.g., yeast cells) capable of producing one or more HMOs encode one or more heterologous nucleic acids encoding one or more enzymes of the HMO biosynthetic pathway.

In some embodiments, a host cell (e.g., yeast cell) of the disclosure is genetically modified so as to express a fucosyltransferase polypeptide having an amino acid sequence of any one of SEQ ID NOS: 1-41, or a biologically active variant that shares substantial identity with the amino acid sequence of any one of SEQ ID NOS: 1-41. In some embodiments, the variant has at least 70%, 75%, 80%, 85%, 86%, 87%, 88%, 89%, 90%, 91%, 92%, 93%, 94%, 95%, 96%, 97%, 98%, 99%, or 100% identity to the amino acid sequence of any one of SEQ ID NOS: 1-41.

In some embodiments, a host cell (e.g., yeast cell) of the disclosure is genetically modified so as to express a GMD polypeptide having an amino acid sequence of any one of SEQ ID NOS: 42-64, or a biologically active variant that shares substantial identity with the amino acid sequence of any one of SEQ ID NOS: 42-64. In some embodiments, the variant has at least 70%, 75%, 80%, 85%, 86%, 87%, 88%, 89%, 90%, 91%, 92%, 93%, 94%, 95%, 96%, 97%, 98%, 99%, or 100% identity to the amino acid sequence of any one of SEQ ID NOS: 42-64.

In some embodiments, a host cell (e.g., yeast cell) of the disclosure is genetically modified so as to express a lactose permease polypeptide having an amino acid sequence of any one of SEQ ID NOS: 65-99, or a biologically active variant that shares substantial identity with the amino acid sequence of any one of SEQ ID NOS: 65-99. In some embodiments, the variant has at least 70%, 75%, 80%, 85%, 86%, 87%, 88%, 89%, 90%, 91%, 92%, 93%, 94%, 95%, 96%, 97%, 98%, 99%, or 100% identity to the amino acid sequence of any one of SEQ ID NOS: 65-99.

In some embodiments, a host cell (e.g., yeast cell) of the disclosure is genetically modified so as to express a fucose synthase polypeptide having an amino acid sequence of any one of SEQ ID NOS: 100-103, or a biologically active variant that shares substantial identity with the amino acid sequence of any one of SEQ ID NOS: 100-103. In some embodiments, the variant has at least 70%, 75%, 80%, 85%, 86%, 87%, 88%, 89%, 90%, 91%, 92%, 93%, 94%, 95%, 96%, 97%, 98%, 99%, or 100% identity to the amino acid sequence of any one of SEQ ID NOS: 100-103.

Enzyme activity can be assessed using any number of assays, including assays that evaluate the overall production of at least one HMO (e.g., LNnT, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, LST a, LST b, LST c, DS-LNT, F-LST a, F-LST b, FS-LNH, FS-LNnH I, or FDS-LNH II) by a host cell (e.g., yeast cell) strain. For example, production yields may be calculated by quantifying sugar input into fermentation tanks and measuring residual levels of input sugars through ion exchange chromatography. Additional methods that may be used to assess HMO production include mass spectrometry.

In some embodiments, a host cell including one or more heterologous nucleic acids encoding a fucosyltransferase, GMD, lactose permease, and/or fucose synthase enzymes described herein increases HMO production, for example, by at least 10%, at least 20%, at least 30%, at least 40%, at least 50%, or greater, when expressed in a host cell (e.g., a yeast strain described herein) as compared to a counterpart host cell of the same strain that does not express the same fucosyltransferase, GMD, lactose permease, and/or fucose synthase enzyme described herein.

In some embodiments, a host cell including one or more heterologous nucleic acids encoding a fucosyltransferase, GMD, lactose permease, and/or fucose synthase enzymes described herein increases the purity of the HMO produced, e.g., by at least 10%, at least 20%, at least 30%, at least 40%, at least 50%, or greater, when expressed in a host cell compared to a counterpart host cell that does not express the same fucosyltransferase, GMD, lactose permease, and/or fucose synthase enzyme described herein.

In some embodiments, a host cell including one or more heterologous nucleic acids encoding a fucosyltransferase, GMD, lactose permease, and/or fucose synthase enzymes described herein decreases undesired byproduct (e.g., DFL) production by at least 10%, at least 20%, at least 30%, at least 40%, at least 50%, or greater, when expressed in a host cell compared to a counterpart host cell of the same strain that does not express the same fucosyltransferase, GMD, lactose permease, and/or fucose synthase enzyme described herein.

In some embodiments, the host cell includes a heterologous nucleic acid encoding a prostate specific antigen-1 (PSA1) protein. In some embodiments, the host cell includes a heterologous nucleic acid encoding a phosphomannomutase SEC53 (SEC53) protein. In some embodiments, the host cell includes a heterologous nucleic acid encoding a uroporphyrinogen decarboxylase Hem12 (HEM12) protein. In some embodiments, the host cell includes a heterologous nucleic acid encoding a SNF1-activating kinase 1 (SAK1) protein. In some embodiments, the host cell includes a heterologous nucleic acid encoding an acetyl-coenzyme A synthetase 1 (ACS1) protein. In some embodiments, the host cell includes a heterologous nucleic acid encoding a cell wall protein DAN1 (DAN1) protein. In some embodiments, the host cell includes a heterologous nucleic acid encoding a pro-neuropeptide Y (NYP1) protein.

Host Cells Capable of Producing Exemplary HMOs and their Precursors

In some embodiments, the host cells of the disclosure are capable of producing one or more HMOs (e.g., LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II) and their precursors. In some embodiments, the host cells are capable of producing 2′-FL. In some embodiments, the host cells are capable of producing 6′-SL. The sections that follow describe host cells that are capable of producing exemplary HMOs, as well as the biosynthetic pathways that are involved in the production of each exemplary HMO.

In some embodiments, the host cells (e.g., yeast cells) of the disclosure are capable of producing the UDP-glucose HMO precursor. The activated sugar UDP-glucose is composed of a pyrophosphate group, the pentose sugar ribose, glucose, and the nucleobase uracil. UDP-glucose is natively produced by yeast cells, and its production levels can be increased with overexpression of, for example, phosphoglucomutase-2 (PGM2) or UTP glucose-1-phosphate uridylyltransferase (UGP1).

In some embodiments, the host cells (e.g., yeast cells) of the disclosure are capable of producing the UDP-galactose HMO precursor. The activated sugar UDP-galactose is composed of a pyrophosphate group, the pentose sugar ribose, galactose, and the nucleobase uracil. UDP-galactose is natively produced by yeast cells, and its production levels can be increased with overexpression of, for example, UDP-glucose-4-epimerase (GAL10).

In some embodiments, the host cells (e.g., yeast cells) of the disclosure are capable of producing the UDP-N-acetylglucosamine HMO precursor. The activated sugar UDP-N-acetylglucosamine consists of a pyrophosphate group, the pentose sugar ribose, N-acetylglucosamine, and the nucleobase uracil. UDP-N-acetylglucosamine is natively produced by yeast cells, and its production levels can be increased with expression of, for example, UDP-N-acetylglucosamine-diphosphorylase, or overexpression of, for example, glucosamine 6-phosphate N-acetyltransferase (GNA1) or phosphoacetylglucosamine mutase (PCM1).

In some embodiments, the host cells (e.g., yeast cells) of the disclosure are capable of producing the GDP-fucose HMO precursor. The activated sugar GDP-fucose consists of a pyrophosphate group, the pentose sugar ribose, fucose, and the nucleobase guanine. GDP-fucose is not natively produced by yeast cells, and its production can be enabled with the introduction of, for example, GDP-mannose 4,6-dehydratase, e.g., from Escherichia coli, and GDP-L-fucose synthase, e.g., from Arabidopsis thaliana.

In some embodiments, the host cells (e.g., yeast cells) of the disclosure are capable of producing the CMP-sialic acid HMO precursor. The activated sugar CMP-sialic acid consists of a pyrophosphate group, the pentose sugar ribose, sialic acid, and the nucleobase cytosine. CMP-sialic acid is not natively produced by yeast cells, and its production can be enabled with the introduction of, for example, CMP-Neu5Ac synthetase, e.g., from Campylobacter jejuni, sialic acid synthase, e.g., from C. jejuni, and UDP-N-acetylglucosamine 2-epimerase, e.g., from C. jejuni.

In some embodiments, the host cells (e.g., yeast cells) of the disclosure are capable of producing 2′-FL. In some embodiments, the host cells (e.g., yeast cells) of the disclosure are capable of producing 6′-SL. In addition to one or more heterologous nucleic acids encoding one or more of the aforementioned enzymes, the host cell may further include one or more heterologous nucleic acids encoding one or more of GDP-mannose 4,6-dehydratase, e.g., from Escherichia coli, GDP-L-fucose synthase, e.g., from Arabidopsis thaliana, α-1,2-fucosyltransferase, e.g., from Helicobacter pylori, and a fucosidase, e.g., an α-1,3-fucosidase. In some embodiments, the fucosyltransferase is from Candidata moranbacterium or Pseudoalteromonas haloplanktis.

In some embodiments, the host cells (e.g., yeast cells) of the disclosure may include a heterologous nucleic acid encoding an enzyme that can catalyze the conversion of GDP-mannose to GDP-4-dehydro-6-deoxy-D-mannose, e.g., a GDP-mannose 4,6-dehydratase. In some embodiments, the GDP-mannose 4,6-dehydratase is from Escherichia coli. Other suitable GDP-mannose 4,6-dehydratase sources include, for example and without limitation, Caenorhabditis elegans, Homo sapiens, Arabidopsis thaliana, Dictyostelium discoideum, Mus musculus, Drosophila melanogaster, Sinorhizobium fredii HH103, Sinorhizobium fredii NGR234, Planctomycetes bacterium RBG_13_63_9, Silicibacter sp. TrichCH4B, Pandoraea vervacti, Bradyrhizobium sp. YR681, Epulopiscium sp. SCG-B11WGA-EpuloA1, Caenorhabditis briggsae, candidatus Curtissbacteria bacterium RIFCSPLOWO2_12_FULL_38_9, Pseudomonas sp. EpS/L25, Clostridium sp. KLE 1755, Nitrospira sp. SG-bin2, Cricetulus griseus, Arthrobacter siccitolerans, and Paraburkholderia piptadeniae. In some embodiments, the GDP-mannose dehydratase is from Caenorhabditis briggsae or Escherichia coli.

In some embodiments, the host cells (e.g., yeast cells) of the disclosure may include a heterologous nucleic acid encoding an enzyme that can catalyze the conversion of GDP-4-dehydro-6-deoxy-D-mannose to GDP-L-fucose, e.g., a GDP-L-fucose synthase. In some embodiments, the GDP-L-fucose synthase is from Arabidopsis thaliana. Other suitable GDP-L-fucose synthase sources include, for example and without limitation, Mus musculus, Escherichia coli K-12, Homo sapiens, Marinobacter salarius, Sinorhizobium fredii NGR234, Oryza sativa Japonica Group, Micavibrio aeruginosavorus ARL-13, Citrobacter sp. 86, Pongo abelii, Caenorhabditis elegans, candidatus Staskawiczbacteria bacterium RIFCSPHIGHO2_01_FULL_41_41, Drosophila melanogaster, Azorhizobium caulinodans ORS 571, candidatus Nitrospira nitrificans, Mycobacterium elephantis, Elusimicrobia bacterium RBG_16_66_12, Vibrio sp. JCM 19231, Planktothrix serta PCC 8927, Thermodesulfovibrio sp. RBG_19FT_COMBO_42_12, Anaerovibrio sp. JC8, Dictyostelium discoideum, and Cricetulus griseus.

In some embodiments, the host cells (e.g., yeast cells) of the disclosure may include a heterologous nucleic acid encoding an enzyme that can catalyze the conversion of GDP-L-fucose and lactose to 2′-FL, e.g., an α-1,2-fucosyltransferase. In some embodiments, the α-1,2-fucosyltransferase is from Helicobacter pylori. In some embodiments, the fucosyltransferase is from Candidata moranbacterium or Pseudoalteromonas haloplanktis ANT/505. Other suitable α-1,2-fucosyltransferase sources include, for example and without limitation, Escherichia coli, Sus scrofa, Homo sapiens, Chlorocebus sabaeus, Pan troglodytes, Macaca mulatta, Oryctolagus cuniculus, Pongo pygmaeus, Mus musculus, Rattus norvegicus, Caenorhabditis elegans, Hylobates lar, Bos taurus, Hylobates agilis, Eulemur fulvus, and Helicobacter hepaticus ATCC 51449. In some embodiments, the source of the α-1,2-fucosyltransferase is Pseudoalteromonas haloplanktis ANT/505, candidatus Moranbacteria bacterium, Acetobacter sp. CAG: 267, Bacteroides vulgatus, Sulfurovum lithotrophicum, Thermosynechococcus elongatus BP-1, Geobacter uraniireducens Rf4, Bacteroides fragilis str. S23L17, Chromobacterium vaccinii, Herbaspirillum sp. YR522, or Helicobacter bilis ATCC 43879.

In some embodiments, the host cells (e.g., yeast cells) of the disclosure may include a heterologous nucleic acid encoding an enzyme that can catalyze the conversion of difucosyllactose to 2′-FL and fucose, e.g., an α1-3,4-fucosidase. Suitable α1-3,4-fucosidase sources include, for example and without limitation, Bacteroides thetaiotaomicron, Bifidobacterium bifidum, Bifidobacterium longum, Bifidobacterium longum subsp. infantis, Clostridium perfringens, Lactobacillus casei, Paenibacillus thiaminolyticus, Pseudomonas putida, Thermotoga maritima, Arabidopsis thaliana, and Rattus norvegicus.

In some embodiments, the host cells (e.g., yeast cells) of the disclosure are capable of producing 3-fucosyllactose. In addition to one or more heterologous nucleic acids encoding one or more of the aforementioned enzymes, the host cell may further include one or more heterologous nucleic acids encoding one or more of GDP-mannose 4,6-dehydratase, e.g., from Escherichia coli, GDP-L-fucose synthase, e.g., from Arabidopsis thaliana, α-1,3-fucosyltransferase, e.g., from Helicobacter pylori, and a fucosidase, e.g., an α-1,2-fucosidase.

In some embodiments, the host cells (e.g., yeast cells) of the disclosure may include a heterologous nucleic acid encoding an enzyme that can catalyze the conversion of GDP-L-fucose and lactose to 3-fucosyllactose, e.g., an α-1,3-fucosyltransferase. In some embodiments, the α-1,3-fucosyltransferase is from Helicobacter pylori. Other suitable α-1,3-fucosyltransferase sources include, for example and without limitation, Homo sapiens, Escherichia coli, Sus scrofa, Chlorocebus sabaeus, Pan troglodytes, Macaca mulatta, Oryctolagus cuniculus, Pongo pygmaeus, Mus musculus, Rattus norvegicus, Caenorhabditis elegans, Hylobates lar, Bos taurus, Hylobates agilis, Eulemur fulvus, Helicobacter hepaticus ATCC 51449, Akkermansia muciniphila, Bacteroides fragilis, and Zea mays.

In some embodiments, the host cells (e.g., yeast cells) of the disclosure are capable of producing lacto-N-tetraose. In addition to one or more heterologous nucleic acids encoding one or more of the aforementioned enzymes, the host cell may further include one or more heterologous nucleic acids encoding one or more of β-1,3-N-acetylglucosaminyltransferase, e.g., from Neisseria meningitidis, β-1,3-galactosyltransferase, e.g., from Escherichia coli, and UDP-N-acetylglucosamine-diphosphorylase, e.g., from E. coli.

In some embodiments, the host cells (e.g., yeast cells) of the disclosure may include a heterologous nucleic acid encoding an enzyme that can catalyze the conversion of UDP-N-acetyl-alpha-D-glucosamine and lactose to lacto-N-triose II and UDP, e.g., a β-1,3-N-acetylglucosaminyltransferase. In some embodiments, the β-1,3-N-acetylglucosaminyltransferase is from Neisseria meningitidis. Other suitable β-1,3-N-acetylglucosaminyltransferase sources include, for example and without limitation, Arabidopsis thaliana, Streptococcus dysgalactiae subsp. equisimilis, Escherichia coli, e.g., Escherichia coli K-12, Pseudomonas aeruginosa PAO1, Homo sapiens, Mus musculus, Mycobacterium smegmatis str. MC2 155, Dictyostelium discoideum, Komagataeibacter hansenii, Aspergillus nidulans FGSC A4, Schizosaccharomyces pombe 972h-, Neurospora crassa OR74A, Aspergillus fumigatus Af293, Ustilago maydis 521, Bacillus subtilis subsp. subtilis str. 168, Rattus norvegicus, Listeria monocytogenes EGD-e, Bradyrhizobium japonicum, Nostoc sp. PCC 7120, Haloferax volcanii DS2, Caulobacter crescentus CB15, Mycobacterium avium subsp. silvaticum, Oenococcus oeni, Neisseria gonorrhoeae, Propionibacterium freudenreichii subsp. shermanii, Escherichia coli O157: H7, Aggregatibacter actinomycetemcomitans, Bradyrhizobium diazoefficiens USDA 110, Francisella tularensis subsp. novicida U112, Komagataeibacter xylinus, Haemophilus influenzae Rd KW20, Fusobacterium nucleatum subsp. nucleatum ATCC 25586, Bacillus phage SPbeta, Coccidioides posadasii, Populus tremula x Populus alba, Rhizopus microsporus var. oligosporus, Streptococcus parasanguinis, Shigella flexneri, Caenorhabditis elegans, Hordeum vulgare, Synechocystis sp. PCC 6803 substr. Kazusa, Streptococcus agalactiae, Plasmopara viticola, Staphylococcus epidermidis RP62A, Shigella phage SfII, Plasmid pWQ799, Fusarium graminearum, Sinorhizobium meliloti 1021, Physcomitrella patens, Sphingomonas sp. S88, Streptomyces hygroscopicus subsp. jinggangensis 5008, Drosophila melanogaster, Phytophthora infestans, Staphylococcus aureus subsp. aureus Mu50, Penicillium chrysogenum, and Tribolium castaneum.

In some embodiments, the host cells (e.g., yeast cells) of the disclosure may include a heterologous nucleic acid encoding an enzyme that can catalyze the conversion of UDP-galactose and lacto-N-triose II to lacto-N-tetraose and UDP, e.g., a β-1,3-galactosyltransferase. In some embodiments, the β-1,3-galactosyltransferase is from Escherichia coli. Other suitable β-1,3-galactosyltransferase sources include, for example and without limitation, Arabidopsis thaliana, Streptococcus dysgalactiae subsp. equisimilis, Pseudomonas aeruginosa PAO1, Homo sapiens, Mus musculus, Mycobacterium smegmatis str. MC2 155, Dictyostelium discoideum, Komagataeibacter hansenii, Aspergillus nidulans FGSC A4, Schizosaccharomyces pombe 972h-, Neurospora crassa OR74A, Aspergillus fumigatus Af293, Ustilago maydis 521, Bacillus subtilis subsp. subtilis str. 168, Rattus norvegicus, Neisseria meningitidis, Listeria monocytogenes EGD-e, Bradyrhizobium japonicum, Nostoc sp. PCC 7120, Haloferax volcanii DS2, Caulobacter crescentus CB15, Mycobacterium avium subsp. silvaticum, Oenococcus oeni, Neisseria gonorrhoeae, Propionibacterium freudenreichii subsp. shermanii, Aggregatibacter actinomycetemcomitans, Bradyrhizobium diazoefficiens USDA 110, Francisella tularensis subsp. novicida U112, Komagataeibacter xylinus, Haemophilus influenzae Rd KW20, Fusobacterium nucleatum subsp. nucleatum ATCC 25586, Bacillus phage SPbeta, Coccidioides posadasii, Populus tremula x Populus alba, Rhizopus microsporus var. oligosporus, Streptococcus parasanguinis, Shigella flexneri, Caenorhabditis elegans, Hordeum vulgare, Synechocystis sp. PCC 6803 substr. Kazusa, Streptococcus agalactiae, Plasmopara viticola, Staphylococcus epidermidis RP62A, Shigella phage SfII, Plasmid pWQ799, Fusarium graminearum, Sinorhizobium meliloti 1021, Physcomitrella patens, Sphingomonas sp. S88, Streptomyces hygroscopicus subsp. jinggangensis 5008, Drosophila melanogaster, Phytophthora infestans, Staphylococcus aureus subsp. aureus Mu50, Penicillium chrysogenum, and Tribolium castaneum.

In some embodiments, the host cells (e.g., yeast cells) of the disclosure may include a heterologous nucleic acid encoding an enzyme that can catalyze the conversion of N-acetyl-α-D-glucosamine 1-phosphate to UDP-N-acetyl-α-D-glucosamine, e.g., a UDP-N-acetylglucosamine-diphosphorylase. In some embodiments, the UDP-N-acetylglucosamine-diphosphorylase is from Escherichia coli.

In some embodiments, the host cells (e.g., yeast cells) of the disclosure are capable of producing lacto-N-neotetraose. In addition to one or more heterologous nucleic acids encoding one or more of the aforementioned enzymes, the host cell may further include one or more heterologous nucleic acids encoding one or more of β-1,3-N-acetylglucosaminyltransferase, e.g., from Neisseria meningitidis, β-1,4-galactosyltransferase, e.g., from N. meningitidis, and UDP-N-acetylglucosamine-diphosphorylase, e.g., from E. coli.

In some embodiments, the host cells (e.g., yeast cells) of the disclosure may include a heterologous nucleic acid encoding an enzyme that can catalyze the conversion of UDP-galactose and lacto-N-triose II to lacto N-neotetraose and UDP, e.g., α-1,4-galactosyltransferase. In some embodiments, the β-1,4-galactosyltransferase is from Neisseria meningitidis. Other suitable β-1,4-galactosyltransferase sources include, for example and without limitation, Homo sapiens, Neisseria gonorrhoeae, Haemophilus influenzae, Acanthamoeba polyphaga mimivirus, Haemophilus influenzae Rd KW20, Haemophilus ducreyi 35000HP, Moraxella catarrhalis, [Haemophilus] ducreyi, Aeromonas salmonicida subsp. salmonicida A449, and Helicobacter pylori 26695.

In some embodiments, the host cells (e.g., yeast cells) of the disclosure are capable of producing 3′-sialyllactose. In addition to heterologous nucleic acids encoding one or more of the aforementioned enzymes, the host cells may further include heterologous nucleic acids encoding CMP-Neu5Ac synthetase, e.g., from Campylobacter jejuni, sialic acid synthase, e.g., from C. jejuni, UDP-N-acetylglucosamine 2-epimerase, e.g., from C. jejuni, UDP-N-acetylglucosamine-diphosphorylase, e.g., from E. coli, and CMP-N-acetylneuraminate-β-galactosamide-α-2,3-sialyltransferase, e.g., from N. meningitides MC58.

In some embodiments, the host cells (e.g., yeast cells) of the disclosure may include a heterologous nucleic acid encoding an enzyme that can catalyze the conversion of UDP-N-acetyl-α-D-glucosamine to N-acetyl-mannosamine and UDP, e.g., a UDP-N-acetylglucosamine 2-epimerase. In some embodiments, the UDP-N-acetylglucosamine 2-epimerase is from Campylobacter jejuni. Other suitable UDP-N-acetylglucosamine 2-epimerase sources include, for example and without limitation, Homo sapiens, Rattus norvegicus, Mus musculus, Dictyostelium discoideum, Plesiomonas shigelloides, Bacillus subtilis subsp. subtilis str. 168, Bacteroides fragilis, Geobacillus kaustophilus HTA426, Synechococcus sp. CC9311, Sphingopyxis alaskensis RB2256, Synechococcus sp. RS9916, Moorella thermoacetica ATCC 39073, Psychrobacter sp. 1501 (2011), Zunongwangia profunda SM-A87, Thiomicrospira crunogena XCL-2, Polaribacter sp. MED152, Vibrio campbellii ATCC BAA-1116, Thiomonas arsenitoxydans, Nitrobacter winogradskyi Nb-255, Raphidiopsis brookii D9, Thermoanaerobacter italicus Ab9, Roseobacter litoralis Och 149, Halothiobacillus neapolitanus c2, Halothiobacillus neapolitanus c2, Bacteroides vulgatus ATCC 8482, Zunongwangia profunda SM-A87, Moorella thermoacetica ATCC 39073, Paenibacillus polymyxa E681, Desulfatibacillum alkenivorans AK-01, Magnetospirillum magneticum AMB-1, Thermoanaerobacter italicus Ab9, Paenibacillus polymyxa E681, Prochlorococcus marinus str. MIT 9211, Subdoligranulum variabile DSM 15176, Kordia algicida OT-1, Bizionia argentinensis JUB59, Tannerella forsythia 92A2, Thiomonas arsenitoxydans, Synechococcus sp. BL107, Escherichia coli, Vibrio campbellii ATCC BAA-1116, Rhodopseudomonas palustris HaA2, Roseobacter litoralis Och 149, Synechococcus sp. CC9311, Subdoligranulum variabile DSM 15176, Bizionia argentinensis JUB59, Selenomonas sp. oral taxon 149 str. 67H29BP, Bacteroides vulgatus ATCC 8482, Kordia algicida OT-1, Desulfatibacillum alkenivorans AK-01, Thermodesulfovibrio yellowstonii DSM 11347, Desulfovibrio aespoeensis Aspo-2, Synechococcus sp. BL107, and Desulfovibrio aespoeensis Aspo-2.

In some embodiments, the host cells (e.g., yeast cells) of the disclosure may include a heterologous nucleic acid encoding an enzyme that can catalyze the conversion of N-acetyl-mannosamine and phosphoenolpyruvate to N-acetylneuraminate, e.g., a sialic acid synthase. In some embodiments, the sialic acid synthase is from Campylobacter jejuni. Other suitable sialic acid synthase sources include, for example and without limitation, Homo sapiens, groundwater metagenome, Prochlorococcus marinus str. MIT 9211, Rhodospirillum centenum SW, Rhodobacter capsulatus SB 1003, Aminomonas paucivorans DSM 12260, Ictalurus punctatus, Octadecabacter antarcticus 307, Octadecabacter arcticus 238, Butyrivibrio proteoclasticus B316, Neisseria meningitidis serogroup B., Idiomarina loihiensis L2TR, Butyrivibrio proteoclasticus B316, and Campylobacter jejuni.

In some embodiments, the host cells (e.g., yeast cells) of the disclosure may include a heterologous nucleic acid encoding an enzyme that can catalyze the conversion of N-acetylneuraminate and CTP to CMP-N-acetylneuraminate, e.g., a CMP-Neu5Ac synthetase. In some embodiments, the CMP-Neu5Ac synthetase is from Campylobacter jejuni. Other suitable CMP-Neu5Ac synthetase sources include, for example and without limitation, Neisseria meningitidis, Streptococcus agalactiae NEM316, Homo sapiens, Mus musculus, Bacteroides thetaiotaomicron, Pongo abelii, Danio rerio, Oncorhynchus mykiss, Bos taurus, Drosophila melanogaster, and Streptococcus suis BM407.

In some embodiments, the host cells (e.g., yeast cells) of the disclosure may include a heterologous nucleic acid encoding an enzyme that can catalyze the conversion of CMP-N-acetylneuraminate and lactose to 3′-siallyllactose and CMP, e.g., a CMP-N-acetylneuraminate-β-galactosamide-α-2,3-sialyltransferase. In some embodiments, the CMP-N-acetylneuraminate-β-galactosamide-α-2,3-sialyltransferase is from N. meningitides MC58. Other suitable CMP-N-acetylneuraminate-β-galactosamide-α-2,3-sialyltransferase sources include, for example and without limitation, Homo sapiens, Neisseria meningitidis alpha14, Pasteurella multocida subsp. multocida str. Pm70, Pasteurella multocida, and Rattus norvegicus.

In some embodiments, the host cells (e.g., yeast cells) of the disclosure are capable of producing 6′-sialyllactose. In addition to one or more heterologous nucleic acids encoding one or more of the aforementioned enzymes, the host cell may further include one or more heterologous nucleic acids encoding one or more of CMP-Neu5Ac synthetase, e.g., from Campylobacter jejuni, sialic acid synthase, e.g., from C. jejuni, UDP-N-acetylglucosamine 2-epimerase, e.g., from C. jejuni, UDP-N-acetylglucosamine-diphosphorylase, e.g., from E. coli, and β-galactoside α-2,6-sialyltransferase, e.g., from Photobacterium sp. JT-ISH-224.

In some embodiments, the host cells (e.g., yeast cells) of the disclosure may include a heterologous nucleic acid encoding an enzyme that can catalyze the conversion of CMP-N-acetylneuraminate and lactose to 3′-sialyllactose and CMP, e.g., a β-galactoside-α-2,6-sialyltransferase. In some embodiments, the β-galactoside-α-2,6-sialyltransferase is from Photobacterium sp. JT-ISH-224. Other suitable β-galactoside-α-2,6-sialyltransferase sources include, for example and without limitation, Homo sapiens, Photobacterium damselae, Photobacterium leiognathi, and Photobacterium phosphoreum ANT-2200.

### Exemplary Host Cell Strains

In some embodiments, the host cell is a yeast cell, such as Saccharomyces cerevisiae. Saccharomyces cerevisiae strains suitable for genetic modification and cultivation to produce HMOs as disclosed herein include, but are not limited to, Baker's yeast, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, CEN.PK, CEN.PK2, and AL-1. In some embodiments, the host cell is a strain of Saccharomyces cerevisiae selected from the group consisting of PE-2, CAT-1, VR-1, BG-1, CR-1, and SA-1. In certain aspects, the strain of Saccharomyces cerevisiae is PE-2. In certain embodiments, the strain of Saccharomyces cerevisiae is CAT-1. In some aspects, the strain of Saccharomyces cerevisiae is BG-1.

In some embodiments, the host cell is Kluyveromyces marxianus. Kluyveromyces marxianus can provide several advantages for industrial production, including high temperature tolerance, acid tolerance, native uptake of lactose, and rapid growth rate. Beneficially, this yeast has sufficient genetic similarity to Saccharomyces cerevisiae such that similar or identical promoters and codon optimized genes can be used among the two yeast species. Furthermore, because Kluyveromyces marxianus has a native lactose permease, it is not necessary to introduce a heterologous nucleic acid to introduce this functionality. In some embodiments, at least a portion of the β-galactosidase gene (LAC4) required for metabolizing lactose is deleted in the genetically modified yeast. Thus, the modified Kluyveromyces marxianus strain is capable of importing lactose without consuming it. In some embodiments, the expression of the β-galactosidase gene in the genetically modified yeast is decreased relative to the expression in wild-type Kluyveromyces marxianus. Thus, the modified Kluyveromyces marxianus strain has reduced consumption of imported lactose.

### Gene Expression Regulatory Elements

In some embodiments, the host cells (e.g., yeast cells) of the disclosure may include a promoter that regulates the expression and/or stability of at least one of the heterologous nucleic acids described herein. In certain aspects, the promoter negatively regulates the expression and/or stability of the at least one heterologous nucleic acid. The promoter can be responsive to a small molecule that may be present in a culture medium containing the host cell. In some embodiments, the small molecule is maltose or an analog or derivative thereof. In some embodiments, the small molecule is lysine or an analog or derivative thereof. Maltose and lysine can be attractive selections for the small molecule as they are relatively inexpensive, non-toxic, and stable.

In some embodiments, the promoter is not responsive to a small molecule that may be present in the culture medium. For example, the promoter may not be responsive to maltose. In some embodiments, one or more of the heterologous nucleic acids are not regulated by a maltose regulon.

In some embodiments, the promoter that regulates expression of a heterologous nucleic acid described herein is a relatively weak promoter, or an inducible promoter. Illustrative promoters include, for example, lower-strength GAL pathway promoters, such as GAL10, GAL2, and GAL3 promoters. Additional illustrative promoters for use in conjunction with the heterologous nucleic acids of the disclosure include constitutive promoters from S. cerevisiae, such as the promoter from the native TDH3 gene. In some embodiments, a lower strength promoter provides a decrease in expression of at least 25%, or at least 30%, 40%, or 50%, or more, when compared to a GAL1 promoter.

Expression of a heterologous nucleic acid molecule described herein may be accomplished by introducing the heterologous nucleic acid into the host cells under the control of regulatory elements that permit expression in the host cell. In some embodiments, the heterologous nucleic acid is an extrachromosomal plasmid. In some embodiments, the heterologous nucleic acid is a chromosomal integration vector that can integrate the nucleotide sequence of interest into the chromosome of the host cell.

Introduction of Heterologous Nucleic Acids into a Host Cell

In some embodiments, a heterologous nucleic acid of the disclosure is introduced into a host cell (e.g., yeast cell) by way of a gap repair molecular biology technique. In these methods, if the host cell has non-homologous end joining (NHEJ) activity, as is the case for Kluyveromyces marxianus, then the NHEJ activity in the host cell can be first disrupted in any of a number of ways. Further details related to genetic modification of host cells (e.g., yeast cells) through gap repair can be found in U.S. Pat. No. 9,476,065, the disclosure of which is incorporated herein by reference in its entirety.

In some embodiments, a heterologous nucleic acid of the disclosure is introduced into the host cell by way of one or more site-specific nucleases capable of causing breaks at designated regions within selected nucleic acid target sites. Examples of such nucleases include, but are not limited to, endonucleases, site-specific recombinases, transposases, topoisomerases, zinc finger nucleases, TAL-effector DNA binding domain-nuclease fusion proteins (TALENs), CRISPR/Cas-associated RNA-guided endonucleases, and meganucleases. Further details related to genetic modification of host cells through site specific nuclease activity can be found in U.S. Pat. No. 9,476,065, the disclosure of which is incorporated herein by reference in its entirety.

### Nucleic Acid and Amino Acid Sequence Optimization

Described herein are specific genes and proteins useful in the methods, compositions, and organisms of the disclosure; however, it will be recognized that absolute identity to such genes is not necessary. For example, changes in a particular gene or polynucleotide including a sequence encoding a polypeptide or enzyme can be performed and screened for activity. Typically, such changes include conservative mutations and silent mutations. Such modified or mutated polynucleotides and polypeptides can be screened for expression of a functional enzyme using methods known in the art. Due to the inherent degeneracy of the genetic code, other polynucleotides which encode substantially the same or functionally equivalent polypeptides can also be used to clone and express the polynucleotides encoding such enzymes.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24:216-8).

Those of skill in the art will recognize that, due to the degenerate nature of the genetic code, a variety of DNA molecules differing in their nucleotide sequences can be used to encode a given heterologous polypeptide of the disclosure. A native DNA sequence encoding the biosynthetic enzymes described above is referenced herein merely to illustrate an embodiment of the disclosure, and the disclosure includes DNA molecules of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties, e.g., charge or hydrophobicity. In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (See, e.g., Pearson W. R., 1994, Methods in Mol. Biol. 25:365-89).

Furthermore, any of the genes encoding an enzyme described herein (or any of the regulatory elements that control or modulate expression thereof) can be optimized by genetic/protein engineering techniques, such as directed evolution or rational mutagenesis, which are known to those of ordinary skill in the art. Such action allows those of ordinary skill in the art to optimize the enzymes for expression and activity in yeast.

In addition, genes encoding these enzymes can be identified from other fungal and bacterial species and can be expressed for the modulation of this pathway. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorpha, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia. coli, Zymomonas mobilis, Staphylococcus aureus, Bacillus spp., Clostridium spp., Corynebacterium spp., Pseudomonas spp., Lactococcus spp., Enterobacter spp., Salmonella spp., or X. dendrorhous.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art can be suitable to identify analogous genes and analogous enzymes. Techniques include, but are not limited to, cloning a gene by PCR using primers based on a published sequence of a gene/enzyme of interest, or by degenerate PCR using degenerate primers designed to amplify a conserved region among a gene of interest. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity, e.g., as described herein or in Kiritani, K., Branched-Chain Amino Acids Methods Enzymology, 1970; then isolating the enzyme with said activity through purification; determining the protein sequence of the enzyme through techniques such as Edman degradation; design of PCR primers to the likely nucleic acid sequence; amplification of said DNA sequence through PCR; and cloning of said nucleic acid sequence. To identify homologous or similar genes and/or homologous or similar enzymes, suitable techniques also include comparison of data concerning a candidate gene or enzyme with databases such as BRENDA, KEGG, or MetaCYC. The candidate gene or enzyme can be identified within the above mentioned databases in accordance with the teachings herein.

### Methods of Producing HMOs

Provided herein are host cells capable of producing one or more HMOs and methods of producing one or more HMOs (e.g., one or more of 2′-FL, LNnT, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II). For example, provided herein are methods for producing 2′-FL. In some embodiments, provided herein are methods of producing 6′-SL. The methods may include, for example, providing a population of host cells (e.g., yeast cells) capable of producing one or more HMOs and subsequently introducing one or more heterologous nucleic acids encoding one or more enzymes of the HMO biosynthetic pathway.

In some embodiments, the host cells of the disclosure are cultured under conditions suitable for the production of a desired HMO. The culturing can be performed in a suitable culture medium in a suitable container, such as a cell culture plate, a flask, or a fermentor. Any suitable fermentor may be used, including, but not limited to, a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric et al., in Ullmann's Encyclopedia of Industrial Chemistry, Sixth Edition, Volume 12, pages 398-473, Wiley-VCH Verlag GmbH & Co. KDaA, Weinheim, Germany. Further, the methods can be performed at any scale of fermentation known in the art to support industrial production of microbial products. Materials and methods for the maintenance and growth of cell cultures are well known to those skilled in the art of microbiology or fermentation science (see, for example, Bailey et al., Biochemical Engineering Fundamentals, second edition, McGraw Hill, New York, 1986). Consideration should be given to appropriate culture medium, pH, temperature, and requirements for aerobic, microaerobic, or anaerobic conditions, depending on the specific requirements of the host cell, the fermentation, and the process.

In some embodiments, the culturing is carried out for a period of time sufficient for the transformed population to undergo a plurality of doublings until a desired cell density is reached. In some embodiments, the culturing is carried out for a period of time sufficient for the host cell population to reach a cell density (OD600) of between 0.01 and 400 in the fermentation vessel or container in which the culturing is being carried out. The culturing can be carried out until the cell density is, for example, between 0.1 and 14, between 0.22 and 33, between 0.53 and 76, between 1.2 and 170, or between 2.8 and 400. In terms of upper limits, the culturing can be carried until the cell density is no more than 400, e.g., no more than 170, no more than 76, no more than 33, no more than 14, no more than 6.3, no more than 2.8, no more than 1.2, no more than 0.53, or no more than 0.23. In terms of lower limits, the culturing can be carried out until the cell density is greater than 0.1, e.g., greater than 0.23, greater than 0.53, greater than 1.2, greater than 2.8, greater than 6.3, greater than 14, greater than 33, greater than 76, or greater than 170. Higher cell densities, e.g., greater than 400, and lower cell densities, e.g., less than 0.1, are also contemplated.

In other embodiments, the culturing is carried for a period of time, for example, between 12 hours and 92 hours, e.g., between 12 hours and 60 hours, between 20 hours and 68 hours, between 28 hours and 76 hours, between 36 hours and 84 hours, or between 44 hours and 92 hours. In some embodiments, the culturing is carried out for a period of time, for example, between 5 days and 20 days, e.g., between 5 days and 14 days, between 6.5 days and 15.5 days, between 8 days and 17 days, between 9.5 days and 18.5 days, or between 11 days and 20 days. In terms of upper limits, the culturing can be carried out for less than 20 days, e.g., less than 18.5 days, less than 17 days, less than 15.5 days, less than 14 days, less than 12.5 day, less than 11 days, less than 9.5 days, less than 8 days, less than 6.5 days, less than 5 day, less than 92 hours, less than 84 hours, less than 76 hours, less than 68 hours, less than 60 hours, less than 52 hours, less than 44 hours, less than 36 hours, less than 28 hours, or less than 20 hours. In terms of lower limits, the culturing can be carries out for greater than 12 hours, e.g., greater than 20 hours, greater than 28 hours, greater than 36 hours, greater than 44 hours, greater than 52 hours, greater than 60 hours, greater than 68 hours, greater than 76 hours, greater than 84 hours, greater than 92 hours, greater than 5 days, greater than 6.5 days, greater than 8 days, greater than 9.5 days, greater than 11 days, greater than 12.5 days, greater than 14 days, greater than 15.5 days, greater than 17 days, or greater than 18.5 days. Longer culturing times, e.g., greater than 20 days, and shorter culturing times, e.g., less than 5 hours, are also contemplated.

In certain embodiments, the production of the one or more HMOs by the population of host cells is inducible by an inducing compound. Such host cells can be manipulated with ease in the absence of the inducing compound. The inducing compound is then added to induce the production of one or more HMOs by the host cells. In other embodiments, production of the one or more HMOs by the host cells is inducible by changing culture conditions, such as, for example, the growth temperature, media constituents, and the like.

In certain embodiments, an inducing agent is added during a production stage to activate a promoter or to relieve repression of a transcriptional regulator associated with a biosynthetic pathway to promote production of one or more HMOs. In certain embodiments, an inducing agent is added during a build stage to repress a promoter or to activate a transcriptional regulator associated with a biosynthetic pathway to repress the production of one or more HMOs, and an inducing agent is removed during the production stage to activate a promoter or to relieve repression of a transcriptional regulator to promote the production of one or more HMOs.

As discussed above, in some embodiments, the host cells may include a promoter that regulates the expression and/or stability of a heterologous nucleic acid described herein. Thus, in certain embodiments, the promoter can be used to control the timing of gene expression and/or stability of proteins.

In some embodiments, when fermentation of a host cell capable of producing a desired HMO is carried out in the presence of a small molecule, e.g., at least about 0.1% maltose or lysine, HMO production is substantially reduced or eliminated. When the small molecule is removed from the fermentation culture medium, HMO production is stimulated. Such a system enables the use of the presence or concentration of a selected small molecule in a fermentation medium as a switch for the production of a HMO. Controlling the timing of non-catabolic compound production so as to occur only when production is desired redirects the carbon flux during the non-production phase into cell maintenance and biomass. This more efficient use of carbon can greatly reduce the metabolic burden on the host cells, improve cell growth, increase the stability of the heterologous genes, reduce strain degeneration, and/or contribute to better overall health and viability of the cells.

In some embodiments, the fermentation method includes a two-step process that utilizes a small molecule as a switch to affect the “off” and “on” stages. In the first step, i.e., the “build” stage, wherein production of the compound is not desired, the host cells are grown in a growth or “build” medium including the small molecule in an amount sufficient to induce the expression of genes under the control of a responsive promoter, and the induced gene products act to negatively regulate production of the non-catabolic compound. In the second step, i.e., the “production” stage, the fermentation is carried out in a culture medium including a carbon source wherein the small molecule is absent or present in sufficiently low amounts such that the activity of a responsive promoter is reduced or inactive. As a result, the production of the desired non-catabolic compound by the host cells is stimulated.

In some embodiments, the culture medium is any culture medium in which a host cell (e.g., yeast cell) can subsist, i.e., maintain growth and viability. In some embodiments, the culture medium is an aqueous medium including assimilable carbon, nitrogen, and phosphate sources. Such a medium can also include appropriate salts, minerals, metals, and other nutrients. In some embodiments, the carbon source and each of the essential cell nutrients are added incrementally or continuously to the fermentation media, and each required nutrient is maintained at essentially the minimum level needed for efficient assimilation by growing cells, for example, in accordance with a predetermined cell growth curve based on the metabolic or respiratory function of the cells, which convert the carbon source to a biomass.

In some embodiments, the method of producing one or more HMOs includes culturing host cells in separate build and production culture media. For example, the method can include culturing the host cells in a build stage, wherein the cells are cultured under non-producing conditions, e.g., non-inducing conditions, thereby producing an inoculum. The inoculum may then be transferred into a second fermentation medium under conditions suitable to induce production of one or more HMOs, e.g., inducing conditions. Steady state conditions may then be maintained in the second fermentation stage so as to produce a cell culture containing one or more desired HMOs.

In some embodiments, the culture medium includes sucrose and lactose. In some embodiments, the carbon sources in the culture medium consist essentially of sucrose and lactose. In some embodiments, the carbon sources in the culture medium consist of sucrose and lactose. In some embodiments, the mass ratio of the sucrose to the lactose is selected to influence, adjust, or control the relative production rates of HMO(s) produced by the yeast cells. Controlling the composition of the produced HMO(s) in this way can advantageously permit the increasing of desired products, the decreasing of undesired products, the targeting of a desired product ratio, and the simplification of downstream product separation processes.

The mass ratio of the sucrose to the lactose in the culture medium can be, for example, between 3 and 40, e.g., between 3 and 25.6, between 7.6 and 29.2, between 11.2 and 32.8, between 14.8 and 36.4, between 18.4 and 40, between 3 and 10, between 3 and 5, or between 3 and 4. In terms of upper limits, the mass ratio of the sucrose to the lactose can be less than 40, e.g., less than 36.4, less than 32.8, less than 29.2, less than 25.6, less than 22, less than 18.4, less than 14.8, less than 11.2, less than 7.6, or less than 5. In terms of lower limits, the mass ratio of the sucrose to the lactose can be greater than 3, e.g., greater than 7.6, greater than 11.2, greater than 14.8, greater than 18.4, greater than 22, greater than 25.6, greater than 29.2, greater than 32.8, or greater than 36.4. Higher ratios, e.g., greater than 40, and lower ratios, e.g., less than 3, are also contemplated.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources in the culture medium is greater than about 0.1 g/L, preferably greater than about 0.25 g/L, and more preferably greater than about 1.0 g/L. In some embodiments, the addition of a nitrogen source to the culture medium beyond a certain concentration is not advantageous for the growth of the yeast. As a result, the concentration of the nitrogen sources in the culture medium can be less than about 20 g/L, e.g., less than about 10 g/L or less than about 5 g/L. Further, in some instances it may be desirable to allow the culture medium to become depleted of the nitrogen sources during culturing.

The effective culture medium can contain other compounds, such as inorganic salts, vitamins, trace metals, or growth promoters. Such other compounds can also be present in carbon, nitrogen or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate and mixtures thereof. Typically, the concentration of phosphate in the culture medium is greater than about 1.0 g/L, e.g., greater than about 2.0 g/L or greater than about 5.0 g/L. In some embodiments, the addition of phosphate to the culture medium beyond certain concentrations is not advantageous for the growth of the yeast. Accordingly, the concentration of phosphate in the culture medium can be less than about 20 g/L, e.g., less than about 15 g/L or less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, e.g., greater than about 1.0 g/L or greater than about 2.0 g/L. In some embodiments, the addition of magnesium to the culture medium beyond certain concentrations is not advantageous for the growth of the yeast. Accordingly, the concentration of magnesium in the culture medium can be less than about 10 g/L, e.g, less than about 5 g/L or less than about 3 g/L. Further, in some instances it may be desirable to allow the culture medium to become depleted of a magnesium source during culturing.

In some embodiments, the culture medium can also include a biologically acceptable chelating agent, such as the dihydrate of trisodium citrate. In such instance, the concentration of a chelating agent in the culture medium can be greater than about 0.2 g/L, e.g., greater than about 0.5 g/L or greater than about 1 g/L. In some embodiments, the addition of a chelating agent to the culture medium beyond certain concentrations is not advantageous for the growth of the yeast. Accordingly, the concentration of a chelating agent in the culture medium can be less than about 10 g/L, e.g., less than about 5 g/L or less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, e.g., within the range of from about 20 mg/L to about 1000 mg/L or in the range of from about 50 mg/L to about 500 mg/L.

The culture medium can also include sodium chloride. Typically, the concentration of sodium chloride in the culture medium is within the range of from about 0.1 g/L to about 5 g/L, e.g., within the range of from about 1 g/L to about 4 g/L or in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the volume of such a trace metal solution added to the culture medium is greater than about 1 mL/L, e.g., greater than about 5 mL/L, and more preferably greater than about 10 mL/L. In some embodiments, the addition of a trace metals to the culture medium beyond certain concentrations is not advantageous for the growth of the host cells (e.g., yeast cells). Accordingly, the amount of such a trace metals solution added to the culture medium may desirably be less than about 100 mL/L, e.g., less than about 50 mL/L or less than about 30 mL/L. It should be noted that, in addition to adding trace metals in a stock solution, the individual components can be added separately, each within ranges corresponding independently to the amounts of the components dictated by the above ranges of the trace metals solution.

The culture media can include other vitamins, such as pantothenate, biotin, calcium, inositol, pyridoxine-HCl, thiamine-HCl, and combinations thereof. Such vitamins can be added to the culture medium as a stock solution that, for convenience, can be prepared separately from the rest of the culture medium. In some embodiments, the addition of vitamins to the culture medium beyond certain concentrations is not advantageous for the growth of the host cells (e.g., yeast cells).

The fermentation methods described herein can be performed in conventional culture modes, which include, but are not limited to, batch, fed-batch, cell recycle, continuous, and semi-continuous. In some embodiments, the fermentation is carried out in fed-batch mode. In such a case, some of the components of the medium are depleted during culture, e.g., during the production stage of the fermentation. In some embodiments, the culture may be supplemented with relatively high concentrations of such components at the outset, for example, of the production stage, so that growth and/or HMO production (e.g., HMO production) is supported for a period of time before additions are required. The preferred ranges of these components can be maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those of ordinary skill in the art, the rate of consumption of nutrient increases during culture as the cell density of the medium increases. Moreover, to avoid introduction of foreign microorganisms into the culture medium, addition can be performed using aseptic addition methods, as are known in the art. In addition, a small amount of anti-foaming agent may be added during the culture.

The temperature of the culture medium can be any temperature suitable for growth of the host cells (e.g., yeast cells). For example, prior to inoculation of the culture medium with an inoculum, the culture medium can be brought to and maintained at a temperature in the range of from about 20° C. to about 45° C., e.g., to a temperature in the range of from about 25° C. to about 40° C., such as from about 28° C. to about 32° C. For example, the culture medium can be brought to and maintained at a temperature of 25° C., 25.5° C., 26° C., 26.5° C., 27° C., 27.5° C., 28° C., 28.5° C., 29° C., 29.5° C., 30° C., 30.5° C., 31° C., 31.5° C., 32° C., 32.5° C., 33° C., 33.5° C., 34° C., 34.5° C., 35° C., 35.5° C., 36° C., 36.5° C., 37° C., 37.5° C., 38° C., 38.5° C., 39° C., 39.5° C., or 40° C.

The pH of the culture medium can be controlled by the addition of acid or base to the culture medium. In such cases, when ammonia is used to control pH, it also conveniently serves as a nitrogen source in the culture medium. In some embodiments, the pH is maintained at from about 3.0 to about 8.0, e.g., at from about 3.5 to about 7.0 or from about 4.0 to about 6.5.

In some embodiments, the host cells (e.g., yeast cells) produce 2′-FL. The concentration of produced 2′-FL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 2′-FL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 2′-FL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 2′-FL can be 40 g/l or greater, e.g., 50 g/l, 60 g/l, 70 g/l, 80 g/l, 90 g/l, or greater. For example, in some embodiments, concentrations of produced 2′-FL in the culture medium can be 100 g/l or greater.

The yield of produced 2′-FL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 2′-FL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNnT. The concentration of produced LNnT in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNnT in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNnT concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNnT can be 40 g/l or greater, e.g., 50 g/l, 60 g/l, 70 g/l, 80 g/l, 90 g/l, or greater. For example, in some embodiments, concentrations of produced LNnT in the culture medium can be 100 g/l or greater.

The yield of produced LNnT on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNnT on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNT. The concentration of produced LNT in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNT in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNT concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNT can be 40 g/l or greater, e.g., 50 g/l, 60 g/l, 70 g/l, 80 g/l, 90 g/l, or greater. For example, in some embodiments, concentrations of produced LNT in the culture medium can be 100 g/l or greater.

The yield of produced LNT on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNT on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce 3-FL. The concentration of produced 3-FL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 3-FL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 3-FL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 3-FL can be 40 g/l or greater, e.g., 50 g/l, 60 g/l, 70 g/l, 80 g/l, 90 g/l, or greater. For example, in some embodiments, concentrations of produced 3-FL in the culture medium can be 100 g/l or greater.

The yield of produced 3-FL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 3-FL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce 6′-SL. The concentration of produced 6′-SL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 6′-SL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 6′-SL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 6′-SL can be 40 g/l or greater, e.g., 50 g/l, 60 g/l, 70 g/l, 80 g/l, 90 g/l, or greater. For example, in some embodiments, concentrations of produced 6′-SL in the culture medium can be 100 g/l or greater.

The yield of produced 6′-SL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 6′-SL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP I. The concentration of produced LNFP I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP I can be 40 g/l or greater, e.g., 50 g/l, 60 g/l, 70 g/l, 80 g/l, 90 g/l, or greater. For example, in some embodiments, concentrations of produced LNFP I in the culture medium can be 100 g/l or greater.

The yield of produced LNFP I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP II. The concentration of produced LNFP II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP II can be 40 g/l or greater, e.g., 50 g/l, 60 g/l, 70 g/l, 80 g/l, 90 g/l, or greater. For example, in some embodiments, concentrations of produced LNFP II in the culture medium can be 100 g/l or greater.

The yield of produced LNFP II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP III. The concentration of produced LNFP III in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP III in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP III concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP III can be 40 g/l or greater, e.g., 50 g/l, 60 g/l, 70 g/l, 80 g/l, 90 g/l, or greater. For example, in some embodiments, concentrations of produced LNFP III in the culture medium can be 100 g/l or greater.

The yield of produced LNFP III on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP III on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP V. The concentration of produced LNFP V in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP V in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP V concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP V can be 40 g/l or greater, e.g., 50 g/l, 60 g/l, 70 g/l, 80 g/l, 90 g/l, or greater. For example, in some embodiments, concentrations of produced LNFP V in the culture medium can be 100 g/l or greater.

The yield of produced LNFP V on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP V on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNFP VI. The concentration of produced LNFP VI in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNFP VI in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNFP VI concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNFP VI can be 40 g/l or greater, e.g., 50 g/l, 60 g/l, 70 g/l, 80 g/l, 90 g/l, or greater. For example, in some embodiments, concentrations of produced LNFP VI in the culture medium can be 100 g/l or greater.

The yield of produced LNFP VI on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNFP VI on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce 3′-SL. The concentration of produced 3′-SL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced 3′-SL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the 3′-SL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced 3′-SL can be 40 g/l or greater, e.g., 50 g/l, 60 g/l, 70 g/l, 80 g/l, 90 g/l, or greater. For example, in some embodiments, concentrations of produced 3′-SL in the culture medium can be 100 g/l or greater.

The yield of produced 3′-SL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of 3′-SL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNDFH I. The concentration of produced LNDFH I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNDFH I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNDFH I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNDFH I can be 40 g/l or greater, e.g., 50 g/l, 60 g/l, 70 g/l, 80 g/l, 90 g/l, or greater. For example, in some embodiments, concentrations of produced LNDFH I in the culture medium can be 100 g/l or greater.

The yield of produced LNDFH I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNDFH I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNDFH II. The concentration of produced LNDFH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNDFH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNDFH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNDFH II can be 40 g/l or greater, e.g., 50 g/l, 60 g/l, 70 g/l, 80 g/l, 90 g/l, or greater. For example, in some embodiments, concentrations of produced LNDFH II in the culture medium can be 100 g/l or greater.

The yield of produced LNDFH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNDFH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNH. The concentration of produced LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNH can be 40 g/l or greater, e.g., 50 g/l, 60 g/l, 70 g/l, 80 g/l, 90 g/l, or greater. For example, in some embodiments, concentrations of produced LNH in the culture medium can be 100 g/l or greater.

The yield of produced LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LNnH. The concentration of produced LNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LNnH can be 40 g/l or greater, e.g., 50 g/l, 60 g/l, 70 g/l, 80 g/l, 90 g/l, or greater. For example, in some embodiments, concentrations of produced LNnH in the culture medium can be 100 g/l or greater.

The yield of produced LNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LNH I. The concentration of produced F-LNH I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LNH I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LNH I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LNH I can be 40 g/l or greater, e.g., 50 g/l, 60 g/l, 70 g/l, 80 g/l, 90 g/l, or greater. For example, in some embodiments, concentrations of produced F-LNH I in the culture medium can be 100 g/l or greater.

The yield of produced F-LNH I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LNH I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LNH II. The concentration of produced F-LNH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LNH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LNH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LNH II can be 40 g/l or greater, e.g., 50 g/l, 60 g/l, 70 g/l, 80 g/l, 90 g/l, or greater. For example, in some embodiments, concentrations of produced F-LNH II in the culture medium can be 100 g/l or greater.

The yield of produced F-LNH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LNH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFL. The concentration of produced DFL in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFL in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFL concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFL can be 40 g/l or greater, e.g., 50 g/l, 60 g/l, 70 g/l, 80 g/l, 90 g/l, or greater. For example, in some embodiments, concentrations of produced DFL in the culture medium can be 100 g/l or greater.

The yield of produced DFL on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFL on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFLNH I. The concentration of produced DFLNH I in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFLNH I in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFLNH I concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFLNH I can be 40 g/l or greater, e.g., 50 g/l, 60 g/l, 70 g/l, 80 g/l, 90 g/l, or greater. For example, in some embodiments, concentrations of produced DFLNH I in the culture medium can be 100 g/l or greater.

The yield of produced DFLNH I on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFLNH I on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFLNH II. The concentration of produced DFLNH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFLNH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFLNH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFLNH II can be 40 g/l or greater, e.g., 50 g/l, 60 g/l, 70 g/l, 80 g/l, 90 g/l, or greater. For example, in some embodiments, concentrations of produced DFLNH II in the culture medium can be 100 g/l or greater.

The yield of produced DFLNH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFLNH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DFLNnH. The concentration of produced DFLNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DFLNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DFLNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DFLNnH can be 40 g/l or greater, e.g., 50 g/l, 60 g/l, 70 g/l, 80 g/l, 90 g/l, or greater. For example, in some embodiments, concentrations of produced DFLNnH in the culture medium can be 100 g/l or greater.

The yield of produced DFLNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DFLNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DF-para-LNH. The concentration of produced DF-para-LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DF-para-LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DF-para-LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DF-para-LNH can be 40 g/l or greater, e.g., 50 g/l, 60 g/l, 70 g/l, 80 g/l, 90 g/l, or greater. For example, in some embodiments, concentrations of produced DF-para-LNH in the culture medium can be 100 g/l or greater.

The yield of produced DF-para-LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DF-para-LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DF-para-LNnH. The concentration of produced DF-para-LNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DF-para-LNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DF-para-LNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DF-para-LNnH can be 40 g/l or greater, e.g., 50 g/l, 60 g/l, 70 g/l, 80 g/l, 90 g/l, or greater. For example, in some embodiments, concentrations of produced DF-para-LNnH in the culture medium can be 100 g/l or greater.

The yield of produced DF-para-LNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DF-para-LNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce TF-LNH. The concentration of produced TF-LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced TF-LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the TF-LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced TF-LNH can be 40 g/l or greater, e.g., 50 g/l, 60 g/l, 70 g/l, 80 g/l, 90 g/l, or greater. For example, in some embodiments, concentrations of produced TF-LNH in the culture medium can be 100 g/l or greater.

The yield of produced TF-LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of TF-LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LST a. The concentration of produced LST a in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LST a in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LST a concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LST a can be 40 g/l or greater, e.g., 50 g/l, 60 g/l, 70 g/l, 80 g/l, 90 g/l, or greater. For example, in some embodiments, concentrations of produced LST a in the culture medium can be 100 g/l or greater.

The yield of produced LST a on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LST a on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LST b. The concentration of produced LST b in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LST b in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LST b concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LST b can be 40 g/l or greater, e.g., 50 g/l, 60 g/l, 70 g/l, 80 g/l, 90 g/l, or greater. For example, in some embodiments, concentrations of produced LST b in the culture medium can be 100 g/l or greater.

The yield of produced LST b on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LST b on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce LST c. The concentration of produced LST c in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced LST c in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the LST c concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced LST c can be 40 g/l or greater, e.g., 50 g/l, 60 g/l, 70 g/l, 80 g/l, 90 g/l, or greater. For example, in some embodiments, concentrations of produced LST c in the culture medium can be 100 g/l or greater.

The yield of produced LST c on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of LST c on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce DS-LNT. The concentration of produced DS-LNT in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced DS-LNT in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the DS-LNT concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced DS-LNT can be 40 g/l or greater, e.g., 50 g/l, 60 g/l, 70 g/l, 80 g/l, 90 g/l, or greater. For example, in some embodiments, concentrations of produced DS-LNT in the culture medium can be 100 g/l or greater.

The yield of produced DS-LNT on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of DS-LNT on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LST a. The concentration of produced F-LST a in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LST a in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LST a concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LST a can be 40 g/l or greater, e.g., 50 g/l, 60 g/l, 70 g/l, 80 g/l, 90 g/l, or greater. For example, in some embodiments, concentrations of produced F-LST a in the culture medium can be 100 g/l or greater.

The yield of produced F-LST a on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LST a on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce F-LST b. The concentration of produced F-LST b in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced F-LST b in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the F-LST b concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced F-LST b can be 40 g/l or greater, e.g., 50 g/l, 60 g/l, 70 g/l, 80 g/l, 90 g/l, or greater. For example, in some embodiments, concentrations of produced F-LST b in the culture medium can be 100 g/l or greater.

The yield of produced F-LST b on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of F-LST b on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce FS-LNH. The concentration of produced FS-LNH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced FS-LNH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the FS-LNH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced FS-LNH can be 40 g/l or greater, e.g., 50 g/l, 60 g/l, 70 g/l, 80 g/l, 90 g/l, or greater. For example, in some embodiments, concentrations of produced FS-LNH in the culture medium can be 100 g/l or greater.

The yield of produced FS-LNH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of FS-LNH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce FS-LNnH. The concentration of produced FS-LNnH in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced FS-LNnH in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the FS-LNnH concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced FS-LNnH can be 40 g/l or greater, e.g., 50 g/l, 60 g/l, 70 g/l, 80 g/l, 90 g/l, or greater. For example, in some embodiments, concentrations of produced FS-LNnH in the culture medium can be 100 g/l or greater.

The yield of produced FS-LNnH on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of FS-LNnH on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

In some embodiments, the host cells (e.g., yeast cells) produce FDS-LNH II. The concentration of produced FDS-LNH II in the culture medium can be, for example, between 1 g/l and 125 g/l, e.g., between 5 g/l and 115 g/l, between 10 g/l and 110 g/l, between 15 g/l and 100 g/l, between 20 g/l and 100 g/l, or between 25 g/l and 100 g/l. In some embodiments, the concentration of produced FDS-LNH II in the culture medium can be, for example, between 5 g/l and 100 g/l, e.g., between 5 g/l and 90 g/l, between 10 g/l and 80 g/l, between 10 g/l and 75 g/l, between 20 g/l and 80 g/l, or between 20 g/l and 80 g/l. In some embodiments, the FDS-LNH II concentration can be greater than 5 g/l, e.g., greater than 8.5 g/l, greater than 12 g/l, greater than 15.5 g/l, greater than 19 g/l, greater than 22.5 g/l, greater than 26 g/l, greater than 29.5 g/l, greater than 33 g/l, or greater than 36.5 g/l. In some embodiments, concentrations of produced FDS-LNH II can be 40 g/l or greater, e.g., 50 g/l, 60 g/l, 70 g/l, 80 g/l, 90 g/l, or greater. For example, in some embodiments, concentrations of produced FDS-LNH II in the culture medium can be 100 g/l or greater.

The yield of produced FDS-LNH II on the sucrose in the culture medium can be, for example, between 0.01 g/g and 0.4 g/g, e.g., between 0.01 g/g and 0.3 g/g, between 0.01 g/g and 0.2 g/g, between 0.02 g/g and 0.2 g/g, between 0.03 g/g and 0.2 g/g, between 0.04 g/g and 0.2 g/g, or between 0.04 g/g and 0.2 g/g. In terms of lower limits, the yield of FDS-LNH II on sucrose can be greater than 0.01 g/g, e.g., greater than 0.02 g/g, greater than 0.03 g/g, greater than 0.04 g/g, greater than 0.05 g/g, greater than 0.06 g/g, greater than 0.07 g/g, greater than 0.08 g/g, or greater than 0.09 g/g. Higher yields, e.g., greater than 0.1 g/g, or greater than 0.15, or greater than 0.2 g/g, are also contemplated. For example, in some embodiments, yields are at least 0.25 g/g, e.g., 0.25 g/g, 0.26 g/g, or greater.

### Fermentation Compositions

Also provided are fermentation compositions including a population of host cells. The host cells may include any of the yeast cells disclosed herein and discussed above. In some embodiments, the fermentation composition further includes at least one HMO. The HMO may be a reducing sugar. In some embodiments, the HMO contains a fucose residue. In some embodiments, the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

### Methods of Recovering HMOs

Also provided are methods of recovering one or more HMOs from a host cell or fermentation composition described herein. The method may include separating at least a portion of a population of host cells from a culture medium. In some embodiments, the separating includes centrifugation. In some embodiments, the separating includes filtration.

The provided recovery methods may further include contacting the separated host cells with a heated wash liquid. In some embodiments, the heated wash liquid is a heated aqueous wash liquid. In some embodiments, the heated wash liquid consists of water. In some embodiments, the heated wash liquid includes one or more other liquids or dissolved solid components.

In some embodiments, the method may further include removing the wash liquid from the host cells. In some embodiments, the removed wash liquid is combined with the separated culture medium and further processed to isolate the one or more HMOs. In some embodiments, the removed wash liquid and the separated culture medium are further processed independently of one another. In some embodiments, the removal of the wash liquid from the yeast cells is accomplished by way of centrifugation. In some embodiments, the removal of the wash liquid from the yeast cells is accomplished by way of filtration.

### Infant Formula

Additionally described herein is an infant formula, particularly an infant formula produced by: (i) culturing any one of the host cells of the disclosure in a culture medium, thereby producing a desired HMO, (ii) extracting the HMO, and (iii) formulating the HMO for administration to an infant human subject. The infant formula may be in a liquid form as a concentrate or a ready-to-drink liquid. Alternatively, the infant formula may be in the form of a dry powder that may be reconstituted by the addition of water. The infant formula may be used as a human milk replacement or supplement. In some embodiments, the infant formula is formulated such that it is suitable for consumption by an infant of less than 2 years of age, such as an infant of 23 months or less, 22 months or less, 21 months or less, 20 months or less, 19 months or less, 18 months or less, 17 months or less, 16 months or less, 15 months or less, 14 months or less, 13 months or less, 12 months or less, 11 months or less, 10 months or less, 9 months or less, 8 months or less, 7 months or less, 6 months or less, 5 months or less, 4 months or less, 3 months or less, 2 months or less, or 1 month or less.

Also provided herein are methods of producing an infant formula using the host cells and fermentation compositions of the disclosure. The methods may include, for example, (i) culturing any one of the host cells of the disclosure in a culture medium, thereby producing a desired HMO, (ii) extracting the HMO, and (iii) formulating the HMO for administration to an infant human subject.

In some embodiments, the infant formula of the disclosure includes one or more HMOs selected from LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, and FDS-LNH II.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Screening of Enzymes for 2′-Fucosyllactose Production

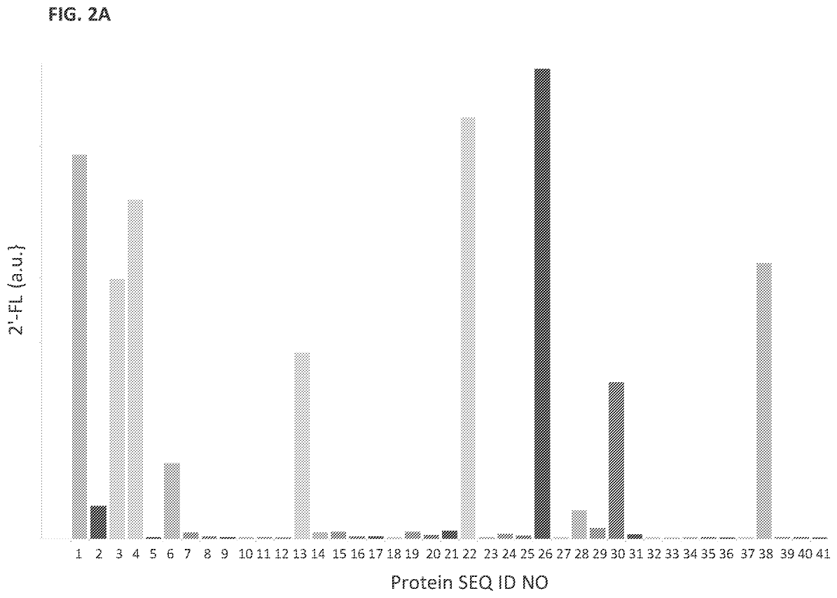

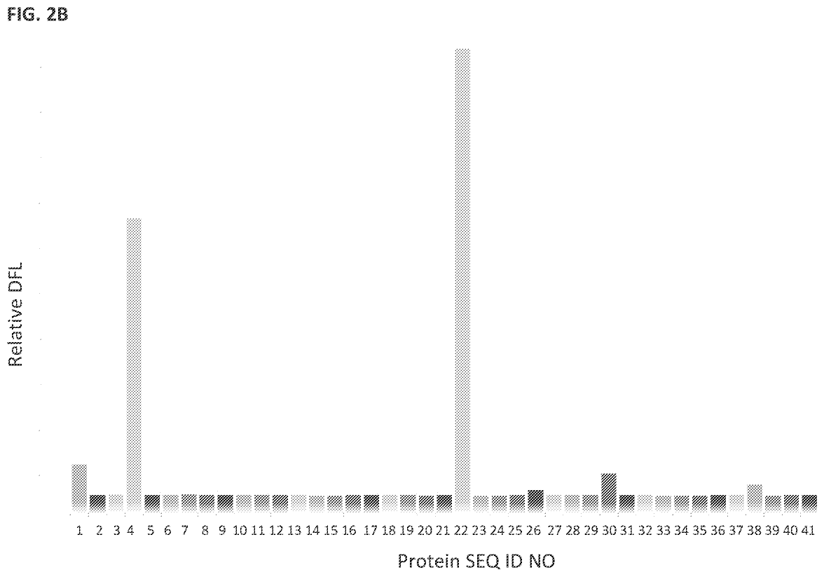

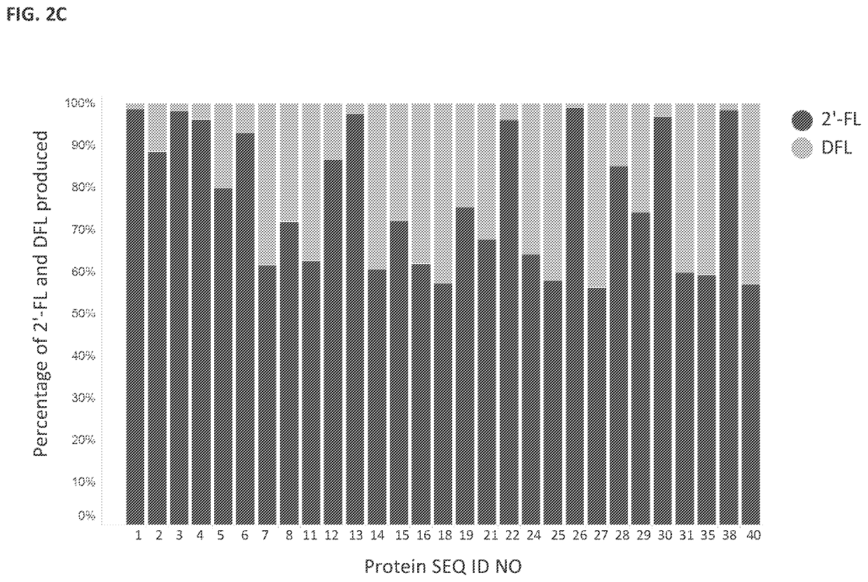

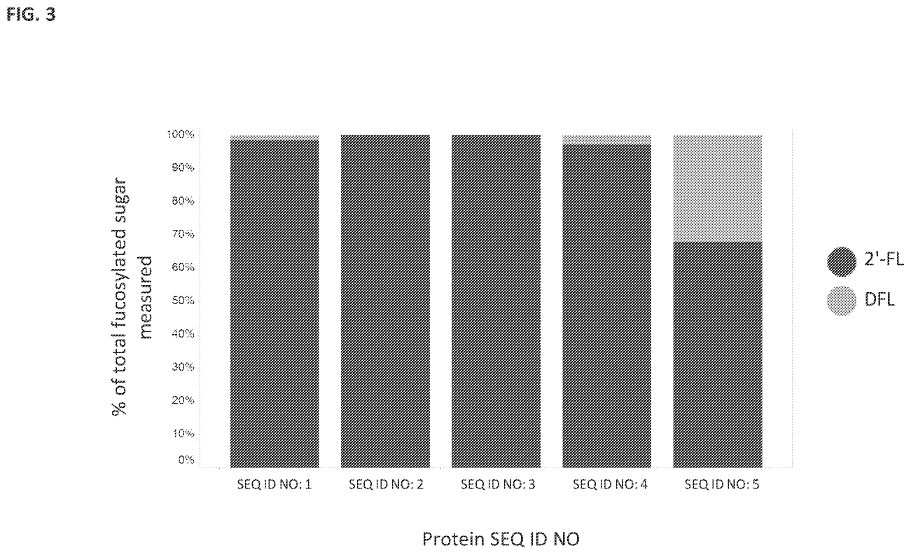

Previously published fucosyltransferases, such as the fucosyltransferases from Helicobacter pylori (SEQ ID NO: 4 and 5), accumulate difucosyllactose (DFL), an undesirable byproduct, in addition to showing the desired activity of producing 2′-fucosyllactose (2′-FL). In this Example, 41 putative fucosyltransferases were identified and expressed separately in a yeast strain capable of lactose uptake and GDP-fucose generation. These strains were grown in microtiter plates and tested for 2′-FL production by mass spectrometry, as described below. Several newly identified enzymes from this screen resulted in the production of 2′-FL with very low levels of DFL byproduct (FIG. 2A-FIG. 2C). The three top-performing strains from the initial diversity search were further characterized via microfermentor screening (FIG. 3) prior to bench-scale tank fermentation screening. Fucosyltransferase enzymes having the amino acid sequences of SEQ ID NOS: 1, 3, and 4 resulted in high 2′-FL production. However, SEQ ID NO: 4 also produced large amounts of DFL byproduct.

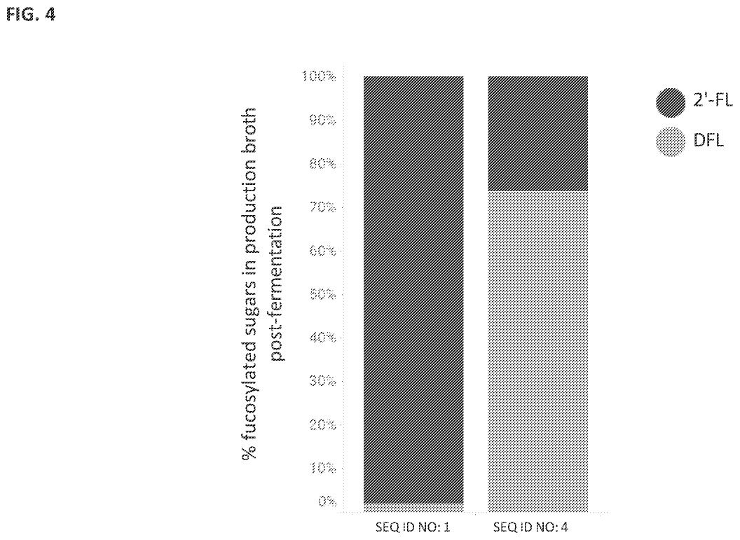

Of the 41 putative fucosyltransferase genes, three new fucosyltransferases were identified that yielded 2′-FL with little to no detectable DFL or 3-FL byproduct formation. The protein sequences for these enzymes were derived from Candidata moranbacterium, Herbaspirillum sp. YR522 and Sulfurovum lithotrophicum from an enzyme diversity search for proteins with putative fucosyltransferase activity. This is the first reported instance of expression of these proteins in a heterologous host or for 2′-FL production. The highly specific product profile of these fucosyltransferases is surprising, particularly since high, specific activity for 2′-FL production was rare in the genes tested (FIG. 2A-FIG. 2C and FIG. 3) as compared to known fucosyltransferase enzymes from H. pylori (SEQ ID NOS: 4 and 5). Bench-scale fermentation with the strain expressing SEQ ID NO: 1 did not generate byproduct fucosylated sugar DFL (FIG. 4). In the same fermentation conditions, strains expressing the H. pylori fucosyltransferase (SEQ ID NO: 4) produced large quantities of DFL.

To assess HMO production, 2′-FL production strains were cultured for 2 days in growth media in 96-well shake plates and diluted into 96-well shake plates containing a sucrose/lactose minimal nutrient medium for oligosaccharide production. Cultures were shaken for 3 days, to sucrose exhaustion, and the wells were extracted, analyzed by mass spectrometer, and quantified by comparison to known standards.

Each putative fucosyltransferase was expressed separately in a yeast base strain capable of lactose uptake and GDP-fucose generation. These strains were tested in batch sugar media in microtiter plates for 2′-FL and DFL production. A subset of these strains were then grown in small scale fed-batch microfermentors to generate the data shown in FIG. 3. In microfermentors, strains received minimal media containing a mixed sucrose to lactose feed at 1:40 ratio w/w at a rate of 2 microliters/hour. After 5 days, whole cell broth from each well was extracted and 2′-FL and DFL were quantified using ion chromatography.

Feeding lactose led to increased DFL titers in the strain expressing the H. pylori fucosyltransferase (SEQ ID NO: 4). The C. moranbacterium fucosyltransferase (SEQ ID NO: 1) continued to produce 2′-FL specifically even as lactose feeding increased and 2′-FL titers rose. Negligible DFL and 3-FL were present in the final product stream, a particularly beneficial result given that these impurities are complicated and expensive to remove via downstream processing. Discovery of this highly specific fucosyltransferase was therefore a significant advancement in generating 2′-FL via fermentation.

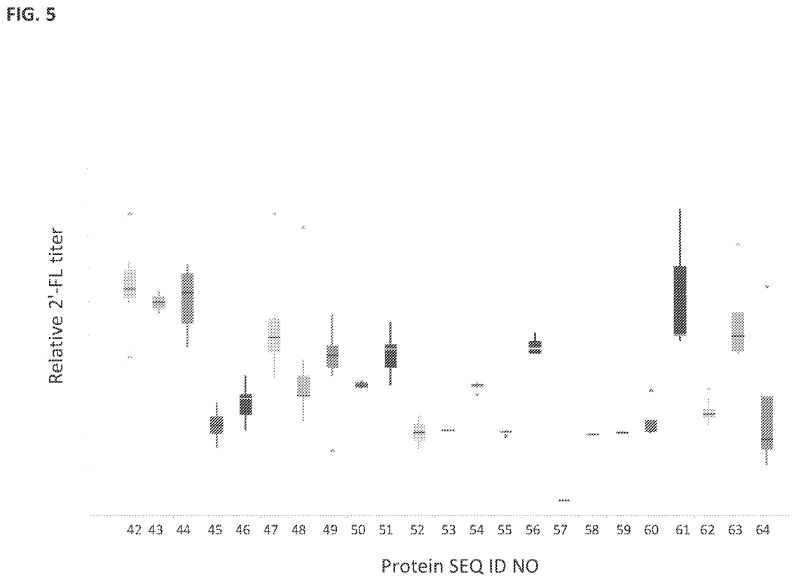

Additionally, 23 GDP-mannose dehydratase (GMD) gene sequences were screened by expressing each individually in a background strain containing GDP-fucose synthetase (GFS), fucosyltransferase, and lactose permease. Several genes were active in S. cerevisiae as measured by 2′-FL production, and three enzymes (SEQ ID NOS: 42, 43, and 44) produced the highest 2′-FL titers (FIG. 5).

The GMD screen was performed by transforming a strain expressing GFS, fucosyltransferase, and lactose permease to express each GMD sequence from the GMD diversity search individually. The resulting strains were assayed in 96-well plates with minimal media containing sucrose and lactose to generate 2′-FL. The three most active candidate enzymes from 96-well plate screening were derived from the organisms E. coli, C. briggsae, and C. elegans with sequences SEQ ID NOS: 42, 43, and 44, respectively.

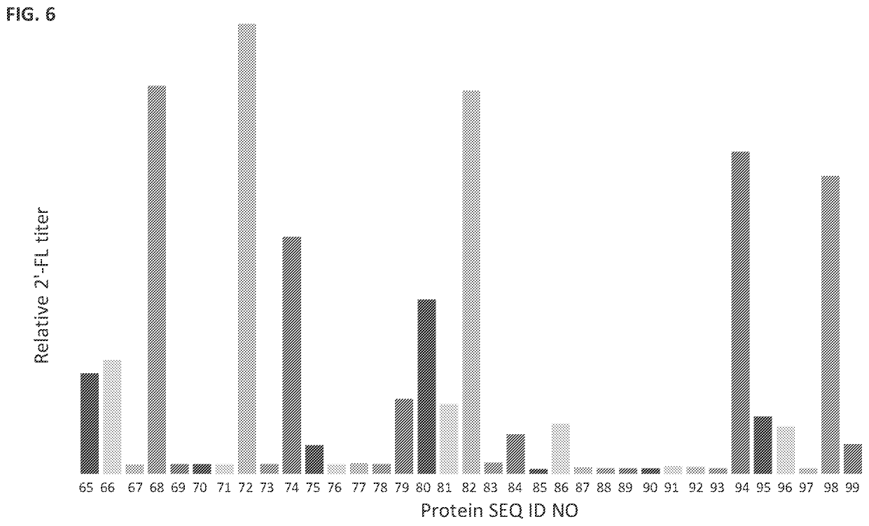

Additionally, 35 putative lactose permease genes were expressed in strains expressing a 2′-FL pathway to assay for lactose uptake via 2′-FL production (FIG. 6). The parent strain did not produce 2′-FL in the absence of a functional lactose permease. Genomic integration was confirmed by PCR.

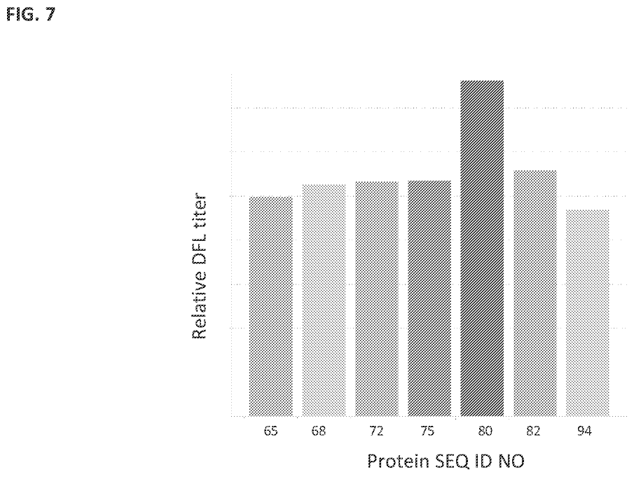

Selected lactose permease genes were expressed in strains containing the 2′-FL biosynthetic pathway with a promiscuous, DFL-producing fucosyltransferase. Strains were fed 0.1% w/v 2′-FL in minimal medium containing no lactose in a 96-well shake plate assay. DFL titer was assessed by mass spectrometry. Strains that took up 2′-FL produced DFL; therefore, DFL titer was used as proxy for 2′-FL uptake. Depending on the permease expressed, more DFL was generated in some instances, indicating a higher affinity for 2′-FL import (FIG. 7). 2′-FL import is metabolically undesirable as it sets up a futile cycle between import and export.

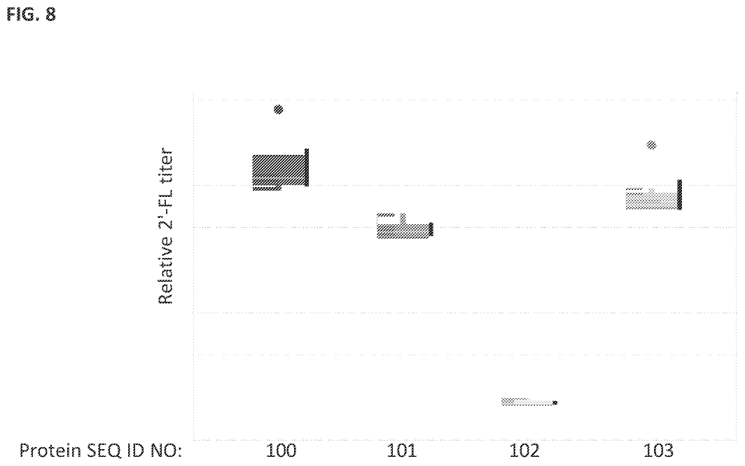

A strain containing GMD, fucosyltransferase, and lactose permease was transformed with each of the candidate FS genes and assessed for resulting 2′-FL production in 96-well microtiter plates. Three out of 4 enzymes, including SEQ ID NOS: 100, 101, and 103, showed FS activity and produced 2′-FL (FIG. 8).

### Materials and Methods

### Yeast Cells

The yeast cells used in these experiments were derived from the well-characterized CEN.PK family of Saccharomyces cerevisiae strains.

### Microtiter Plate Growth Conditions

Pre-culture growth: Strains were incubated in an aerobic, pre-culture, 96-well, 1.1-ml microtiter shakeplate at 28° C., shaking at 1,000 RPM for 48h to reach carbon exhaustion. Pre-culture media conditions were 360 μl/well of minimal complete media with 2% carbon (1.9% maltose+0.1% glucose) with 1 g/L Lysine.

Production growth: After pre-culture, strains were diluted ˜10× (14.4 μl) into a 130 μl/well, 96-well, 1.6-ml microtiter shakeplate containing 4% sucrose+0.1% or 0.5% lactose. Production plates were incubated at 33.5° C. shaking at 1,000 RPM for 72h.

### Extraction and Analysis Conditions for Mass Spectrometry

After 4 days in production conditions, carbon-exhausted whole cell broth was extracted in mass spectroscopy-grade methanol and H₂O. First, 225 μl/well of methanol (5× dilution) was added, with 15 minutes of shaking at 1500 RPM. Next, 900 μl/well of H₂O (21×) was added, followed by shaking for an additional 5 minutes at 1200 RPM. Plates were centrifuged for 5 minutes at 2000 rpm, and 6 μl/well of the top layer were added to a new 1.1-ml plate containing 294 μl/well of 30% MeOH containing xylotriose ISTD (50×). The total dilution from whole cell broth is 880×.

### Extraction and Analysis Conditions for Ion Chromatography

After 4 days in production conditions, carbon-exhausted whole cell broth was extracted using a hot water extraction. Following the addition of 300 μl/well of sterile water (5×), plates were heated and mixed at 1000 RPM for 30 minutes. Plates were then centrifuged for 5 minutes at 2000 RPM. Finally, 25 μl/well of the supernatant is added to 175 μl/well of sterile water into a 1.6 ml plate (8×). Total dilution from whole cell broth was 40×.

This method was intended for quantitation of 2′-FL and DFL in g/kg, in samples of fermentation broth by ion chromatography. 2′-FL and DFL were quantified by using external calibration and ion chromatography pulse amperometric detection with a Dionex CarboPac™ PA1 column.

### Example 2. Modified Yeast Strains for Producing HMOs

Using the compositions and methods described herein, host cells (e.g., yeast cells) can be engineered to produce a HMO, such as lacto-N-neotetraose (LNnT), 2′-fucosyllactose (2′-FL), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, or fucosyl-disialyllacto-N-hexaose (FDS-LNH) II, among others.

To produce the desired HMO, the yeast cell may be genetically modified by introducing into the cell one or more heterologous nucleic acids encoding a fucosyltransferase, a GMD, a lactose permease, and/or a fucose synthase. The fucosyltransferase may have, for example, an amino acid sequence that is at least 90% identical to the amino acid sequence of any one of SEQ ID NOS: 1-41. The GMD may have, for example, an amino acid sequence that is at least 90% identical to the amino acid sequence of any one of SEQ ID NOS: 42-64. The lactose permease may have, for example, an amino acid sequence that is at least 90% identical to the amino acid sequence of any one of SEQ ID NOS: 65-99. The fucose synthase may have, for example, an amino acid sequence that is at least 90% identical to the amino acid sequence of any one of SEQ ID NOS: 100-103. The one or more heterologous nucleic acids encoding the fucosyltransferase, a GMD, a lactose permease, and/or fucose synthase may be integrated into the genome of the yeast cell or they may be present within one or more plasmids.

The yeast cells may be further engineered to express a β-1,3-N-acetylglucosaminyltransferase (LgtA), a β-1,4-galactosyltransferase (LgtB), a fucosidase, a lactose transporter, and/or a UDP-N-acetylglucosamine diphosphorylase. The LgtA may have an amino acid sequence having at least 85% sequence identity to any one of SEQ ID NOS: 104-120. The LgtB may have an amino acid sequence having at least 85% sequence identity to SEQ ID NO: 121 or 122. The heterologous nucleic acids introduced into the yeast cell may be driven by an inducible promoter or may be negatively regulated by the activity of a promoter that is responsive to a small molecule.

The engineered yeast cells may be cultured under conditions suitable for the production of a desired HMO in a suitable culture medium and in a suitable container, for example, a cell culture plate, a flask, or a fermentor. The culturing may be carried out for a period of time sufficient for the transformed population of yeast cells to undergo a plurality of doublings until a desired cell density is reached, for example, the period of time required for the yeast cell population to reach a cell density (OD600) of between 0.01 and 400.

### Example 3. Engineered Yeast Strains Capable of Producing HMOs for Infant Formula

The yeast strains described in Example 2 may be used to produce one or more HMOs that can, in turn, be incorporated into an infant formula. Suitable HMOs for use in an infant formula of the disclosure include, without limitation, LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, and FDS-LNH II.

The infant formula may be produced by culturing host cells as described in Example 2 in a culture medium, thereby producing a desired HMO. The HMO produced may then be extracted and formulated for administration to an infant human subject. The infant formula may be formulated in a liquid form, as a concentrate, or a as a ready-to-drink liquid. Alternatively, the infant formula may be formulated as a dry powder that may be reconstituted by the addition of water. The infant formula may be used as a human milk replacement or supplement. Exemplary infant formulas produced in accordance with this Example may are those that are suitable for consumption by an infant of less than 2 years of age. For example, the infant formula may be formulated for consumption by an infant of 23 months, 22 months, 21 months, 20 months, 19 months, 18 months, 17 months, 16 months, 15 months, 14 months, 13 months, 12 months, 11 months, 10 months, 9 months, 8 months, 7 months, 6 months, 5 months, 4 months, 3 months, 2 months, 1 month of age, or less.

### Example 4. Overexpression of Proteins to Improve Strain Health and Fermentation Yield and Productivity

These experiments were aimed at increasing expression of genes in the native GDP-mannose biosynthesis pathway to improve cell health and strain stability in fermentation. Overexpression of prostate specific antigen-1 (PSA1) and phosphomannomutase SEC53 (SEC53) are both proteins in the GDP-mannose pathway were tested to observe effects on strain health productivity. IMD3 and GUA1, genes from the GTP regeneration pathway, also were overexpressed and showed improved strain performance in earlier lineages when tested in a strain with additional YOR1.

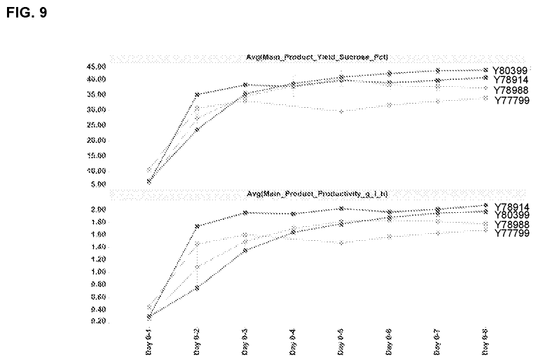

Two genes in the native yeast GDP-mannose biosynthetic pathway PSA1 and SEC53 were upregulated by overexpression. Strains with these genes overexpressed were consistently better performers than their parent strains (FIG. 9).

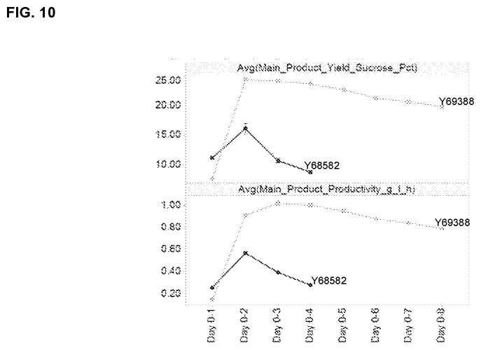

Overexpression of IMD3, GUA1 and additional YOR1 also showed improved strain performance (FIG. 10). The parent strain (Y68582) crashed early in fermentation under the same conditions as its child with additional copies of these native genes (Y69388).

### Example 5. Engineering for Higher Respiration and ATP Levels Improves Fermentation Yield and Productivity

Engineering aimed at increasing ATP levels and pushing metabolism toward respiratory growth was performed to increase 2′-FL production. The specific engineering that was performed that improved strain performance included HEM12 and SAK1 overexpression as well as downregulation of ROX1 through promoter swap (Tables 2 and 3).

Overexpression of HEM3/HEM12 and deletion/downregulation of ROX1 was tested in an effort to improve 2′-FL production. In plates, these strains did not show improvement in 2′-FL titer compared to their parent. However, some strains such as pGAL1>HEM12, ROX1 deletion and pSLN1>ROX1 showed higher ssOD in 1% lactose PR plates.

HEM12 and SAK1 overexpression were tested in multiple lineages and showed repeated benefits (Error! Reference source not found.). In the earlier lineage, HEM12 alone improved performance and fermentation stability (Y74452). In the later lineage, HEM12 alone did not improve performance but when overexpressed together with SAK1, tank stability and performance improved (Y80019).

SAK1 overexpression alone was not tested in the later lineage due to time constraints.

TABLE 2
Engineered strains with HEM12 and SAK1 overexpression

| Strain | Genotype | Hermes |
|---|---|---|
| Y74452 | | H11190 |
| Y76837 | Y74452 + pGAL1 > HEM12 | H11190 |
| Y76828 | | H11484 |
| Y78836 | Y76828 + HEM12 o/e | H11484 |
| Y78914 | | H11672 |
| Y80018 | Y78914 + HEM12 o/e | H11672 |
| Y80019 | Y78914 + HEM12 + SAK1 | H11672 |
| | o/e | |

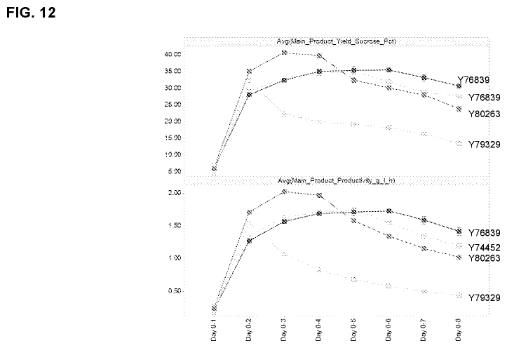

Exchanging the native promoter on ROX1 with pSLN1, an assumed downregulation, also showed repeated benefit in tanks (FIG. 12).

TABLE 3
Engineered strains with ROX1 and pSLN1

| Strain | Genotype | Hermes |
|---|---|---|
| Y74452 | | H11190 |
| Y76839 | Y74452 + pROX1::pSLN1 | H11190 |
| Y76828 | | H11484 |
| Y78837 | Y76828 + pROX1::pSLN1 | H11484 |
| Y79329 | | H11703 |
| Y80263 | Y79329 + pROX1::pSLN1 | Y11703 |

### Example 6. Deletion of MAL Regulon and Mal23 Knockout Greatly Increase Cell Density in Fermentation

Identification and elimination of overexpressed proteins/pathways that are not beneficial for 2′-FL or biomass production benefits cell health and fermentation performance. More specifically, 1) sugar uptake and metabolism genes are upregulated by lactose feeding due to molecular crosstalk (i.e., maltose and lactose are structurally very similar) but do not lead to increased biomass or 2′-FL production and 2) elimination of the maltose and isomaltose genes frees up additional cellular resources for ATP generation.

The maltose switch was removed from the strains. This allowed for the deletion of the maltose regulon expression, which could decrease protein burden from the tremendous upregulation of the MAL regulon, and potentially generate other physiological gains related to decreasing uncleaved-sucrose uptake, shown previously to waste ATP and skew metabolism to a more fermentative state.

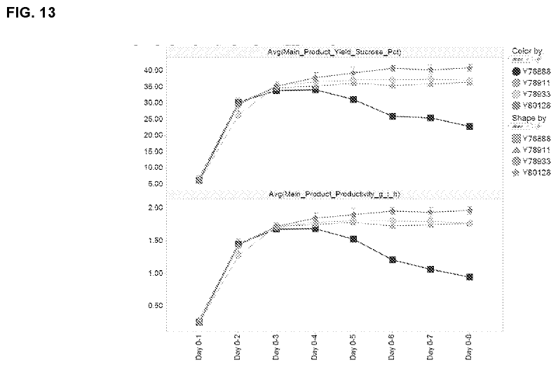

The main benefit of deletion of the MAL regulon was a pronounced boost in cell health (FIG. 13). This engineering, when combined with expression of the YOR1 transport variants, generated very healthy strains with high cell density and good OUR throughout the 8-day fermentation, at the most aggressive feed-rates. The only issue with these strains is that they continued to leave a basal level of lactose in the medium.

A strain with MAL11 and MAL13 deleted (Y78933) has substantially higher cumulative 0-8 day yield (37.0% g 2′-FL/g sucrose compared to 22.8%, a 62% increase) and productivity (1.77 g/L/hr compared to 0.94 g/L/hr, an 88% increase) than its parent strain (Y76888, Error! Reference source not found. 13). Deletion of these genes greatly reduced expression of the maltose regulon and isomaltase genes in CEN.PK yeast; this strain experienced little to no growth with maltose as its sole carbon source. Expression of a specifically engineered 2′-FL export protein (YOR1 I1127A) improved performance of both strain lineages, with the MAL regulon deletion lineage (Y80128) continuing to outperform the strain with intact maltose genes (Y78911).

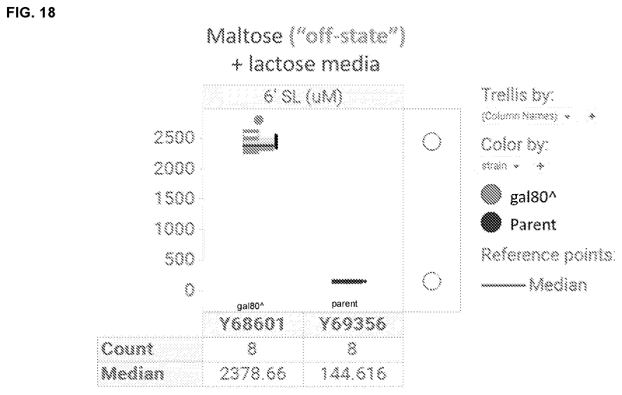

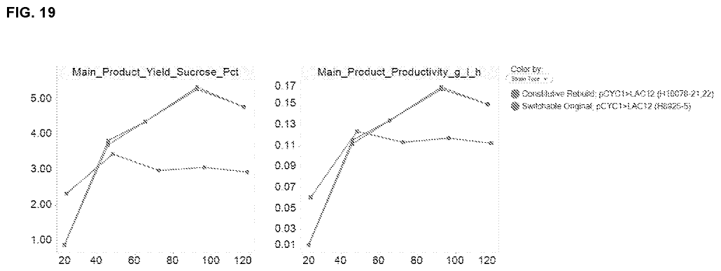

In a 6′-SL producing strain, deletion of the MAL regulon showed similar benefits in terms of improvements in yield and productivity (FIG. 18, FIG. 19).

### Example 7. Overexpression of ACS1 and DAN1

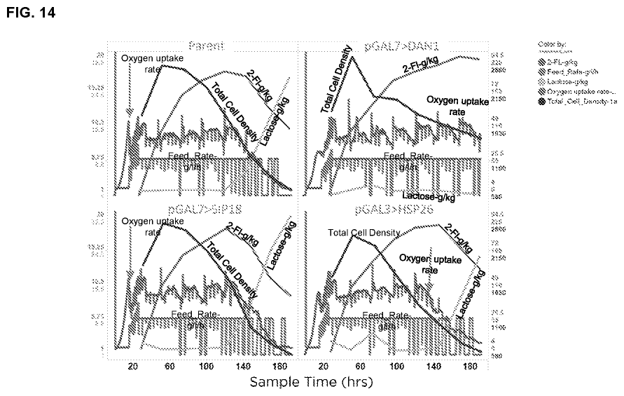

The first experiment performed in an effort to increase conversion of acetate to acetyl-CoA was related to overfeeding lactose and taking RNA-seq measurements as lactose concentration was ramped in the medium. As lactose was still ramping and the culture was healthy, but being challenged by increasing lactose, the differential expression of genes between earlier and later time points led to identification of several genes that are upregulated very highly as lactose levels become problematic (Error! Reference source not found. 4). All of these highly upregulated genes could be part of an osmotic pressure response. The genes in Error! Reference source not found.4 were overexpressed in a top strain and although all the leads generated 2′-FL gains in 96-well plate screening, only DAN1 overexpression translated to tanks. DAN1 overexpression, however, generated cell health gains in a number of lineages, exemplified in FIG. 14.

TABLE 4
Highly upregulated genes across multiple lactose challenge fermentation experiments

| | | Gene¤ | Description¤ | Biological·Role¤ |
|---|---|---|---|---|
| Log2-fold·change¤ | [symbol] | SIP18¤ HSP26¤ GRE1¤ DAN1¤ | Phospholipid-binding·hydrophilin; ·To·overcome· desiccation-rehydration; ·induced·by·osmotic·stress; ·paralog·of·GRE1¤ Small·heat·shock·protein· (sHSP)·with·chaperone·activity; ·suppresses·unfolded· protein·aggregation¤ Hydrophilin·essential·in· desiccation-rehydration; stress·induced; regulated·by· HOG·pathway; ·paralog· of·SIP18¤ Cell·wall·mannoprotein, ·normally·expressed·under· anaerobic·conditions¤ | Osmoregulation·and· balancing¤ Protect·proteins·during· stress¤ Osmoregulation·and· balancing¤ Unknown, ·cells-surface composition¤ |

Another experiment was performed by comparing the transcriptional response (RNA-seq) of strains at pH 5, 5.5 and 6. When the transcriptome of the less healthy pH 5-grown strains to pH 5.5 or pH 6-grown strains were compared, a common gene-enrichment signature emerged. Statistical clustering to identify over-represented differentially expressed biological processes and the specific genes being affected, was carried out.

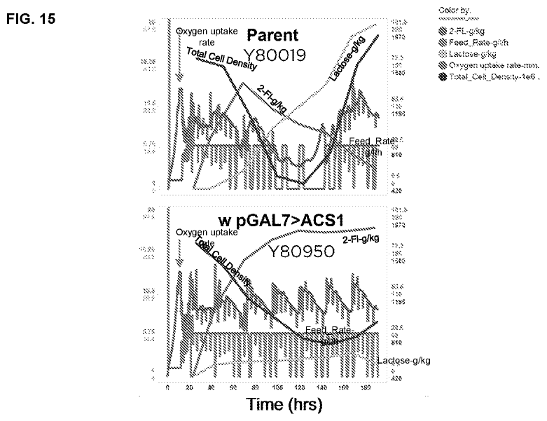

Table 5Error! Reference source not found. shows the major upregulated biological processes when the transcript abundance of strains grown in pH 5 were compared to pH 5.5. The major processes affected were related to small organic acids and metabolites, along with secondary metabolism and pull towards acetyl-CoA-associated processes. The bolded gene, ACS1, was an integral node in most of the enriched upregulated gene clusters and was chosen for upregulation, among other leads. ACS1 was of particular interest due to its role in acetic acid metabolism, it was noted that up to 40 g/L of acetic acid would accumulate as the fermentation cultures died and this observation, along with the ATP requirement of generating acetyl-CoA from acetate, made engineering this node attractive. Upregulation of ACS1 generated a marked increase in cell health as exemplified by the data in FIG. 15Error! Reference source not found.

TABLE 5
Over-represented upregulated biological processes by
gene between pH 5 and pH 5.5-grown cultures.

| Biological process | Genes |
|---|---|
| Acetate metabolism | ACS1, STL1, ADY2, ENA1, FAA1, |
| | JEN1 |
| Pyruvate metabolism | ACS1, ADY2, CAT2, JEN1, DLD1 |
| Oleic acid metabolism | STL1, PXA2, CAT2, FAA1 |
| Glyoxylate metabolism | ACS1, STL1, JEN1, CAT2 |
| Ethanol response | ACS1, DLD1, ENA1, FAA1, JEN1 |
| Fatty acid beta oxidation | PXA2, CAT2, FAA1, YEF1 |
| Glycerol metabolism | ENA1, STL1, JEN1, DLD1 |
| Coenzyme_A metabolism | ACS1, PXA2, CAT2, FAA1 |

### Example 7. Overexpression of NADH Diphosphatase Gene NPY1 Improved Cell Health and Fermentation Performance

A native gene overexpression library with 283 designs (243 unique genes) was screened in strain Y77307. Each design overexpressed just one gene natively found in S. cerevisiae. From the initial plate screening data, 21 hits were banked and 4 were run in AMBRs in the strain H11470. There was one clear hit in the pGAL1>NPY1 design with improvements in strain health, productivity, and yield over both parent Y77307 and process control Y74452.

283 designs containing overexpression of 243 S. cerevisiae genes were compiled from 4 overexpression libraries. Four top hits were run in H11470 at n=2 replication including:
Y78300-pGAL1>NPY1,
Y78309-pGAL7>RFT1,
Y78306-pATP18>FZO1, and
Y78310-pATP18>OLE1.
   The experiments were performed using the AMBR Process of QUESST v3 with sucrose only and at a pH 6 with the strains described in Table 6.

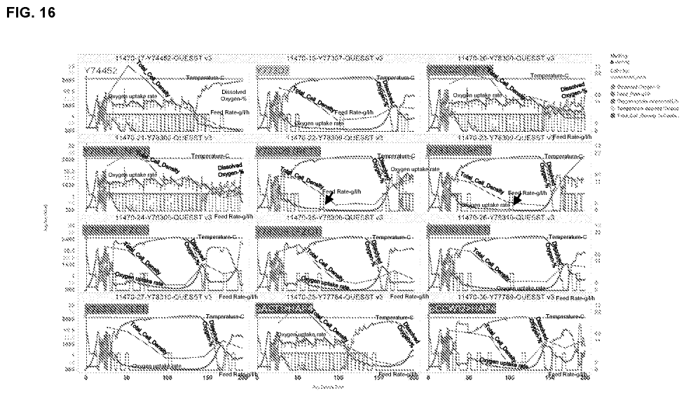

Overexpression of the NADH diphosphatase gene NPY1 was a hit when tested in the fermentation tank. The experiment showed that pGAL1>NPY1 rescues Y77307 from crashing in QUESST v3 conditions (Error! Reference source not found. Y78300) (FIG. 16). The yield and productivity for Y78300 (pGAL1>NPY1) were higher than both parent and process control Y74452 (FIG. 16) Error! Reference source not found. The parent strain Y77307 crashed immediately after growth pulses, which was unexpected given previous runs with its parent Y77163. Y77307 was a plasmid-cured version of Y77163. The results showed that NYP1 improved cell health and fermentation performance the most in comparison to the other designs. This was surprising given that 2′-FL vs SSOD plate data from the overexpression library did not show any indication that NPY1 would perform so much better than the other hits in tanks.

TABLE 6
Top strains nominated for AMBR tanks.

| | | | | | | 2′-FL | SSOD |
|---|---|---|---|---|---|---|---|
| | | | | | | normalized | normalized |
| Y# | Parent | Gene | Promoter | Terminator | Keywords | to Y77307 | to Y77307 |
| Y78300 | Y77307 | NPY1 | pGAL1_24trunc | tTIP1_m2k | NADH | 1.71 | 0.93 |
| | | | | | pathway, | | |
| | | | | | cytoplasmic | | |
| Y78309 | Y77307 | RFT1 | pGAL7_24trunc | tSDH3 | Protein | 1.39 | 0.93 |
| | | | | | secretion | | |
| Y78304 | Y77307 | DGA1 | pGAL1_24trunc | native | Increases | 1.49 | 0.88 |
| | | | | terminator | lipid | | |
| | | | | | accumulation | | |
| Y78298 | Y77307 | PAN5 | pGAL1_24trunc | tTIP1_m2k | Pantothenate / | 1.99 | 0.79 |
| | | | | | CoA | | |
| | | | | | biosynthesis | | |
| Y78299 | Y77307 | MCR1 | pGAL1_24trunc | tTIP1_m2k | Mitochondrial | 1.96 | 0.77 |
| | | | | | NADH | | |
| | | | | | pathway | | |
| Y78306 | Y77307 | FZO1 | pATP18 | native | Mitochondrial | 1.46 | 0.98 |
| | | | | terminator | fusion | | |
| | | | | | (strong | | |
| | | | | | promoters | | |
| | | | | | made cells | | |
| | | | | | sick) | | |
| Y78302 | Y77307 | MGM1 | pGAL3_24trunc_{—} | native | Mitochondrial | 1.50 | 1.01 |
| | | | ase | terminator | fusion | | |
| | | | | | (strong | | |
| | | | | | promoters | | |
| | | | | | made cells | | |
| | | | | | sick) | | |
| Y78310 | Y77307 | OLE1 | pATP18 | native | Membrane | 1.17 | 1.43 |
| | | | | terminator | fluidity | | |
| Y78305 | Y77307 | OLE1 | pGAL1_24trunc | tTIP1_m2k | Membrane | 1.47 | 0.68 |
| | | | | | fluidity | | |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

SEQUENCE APPENDIX

SEQ ID NO: 1 A0A0G0EK12-Fucosyltransferase
MIIVKLMGGMGNQLFQYAIGRSLAIRNESEFKMDILGYADQGELLTPRLYALNIFSVQENFASEKEIEKL
KSNTSGSVVLALQRFGFFKKSNSFVIEPHFNFSSEILESGNNIYLQGYWQTEKYFKDVEDVIRKEFTLK
EKFSIEEKEITKEIKNSNAVSLHIRRGDYVSSATTSKFHGICSLDYYEKAVRHIAEKTENPVFYIFSDDIA
WVKENLKIDFPTKYVSDGILKDYEELTLMSYCKHNIIANSSFSWWGAWLNANPEKIVIAPKQWFADQS
VNTSDVVPETWVKM
SEQ ID NO: 2 J2V5D8-Fucosyltransferase
MIATRLIGGLGNQMFQYAAGRALALRVGSPLLLDVSGFANYELRRYELDGFRIDATAASAQQLARLGV
NATPGTSLLARVLRKVWPQPADRILREASFTYDARIEQASAPVYLDGYWQSERYFARIRQHLLDEFTL
KGDWGSDNAAMAAQIATAGAGAVSLHVRRGDYVSNAHTAQYHGVCSLDYYRDAVAHIGGRVEAPH
FFVFSDDHEWVRENLQIGHPATFVQINSADHGIYDMMLMKSCRHHIIANSSFSWWGAWLNPAEDKIV
VAPQRWFKDATNDTRDLIPAAWVRL
SEQ ID NO: 3 A0A0F6Z144-Fucosyltransferase
MIIINILGGLGNQMFQYAFAYSMAHKTDAVVKLDIEDFSNYDLREYELSLYNISLDLADIDEIDKLKYEQE
TLFKKVARKLQRTSRPLSSYYYKESGFSYDSHVYELKDNVYFQGYWQSEKYFLDYRDALLKEFLLKD
GLHQESRAYEKKINQSVSVSLHIRRGDYVSNAHTNSVHGTCSLEYYKGAVRYLQSNSHPTHFFIFSD
DLDWAKENLNFIENITFVSLDKDTPDHEEMYLMSQCKHNIIANSSFSWWGAWLNQNEDKIVVAPKKW
FNDTTINTNDLVPKEWIRL
SEQ ID NO: 4 Q9X3N7-Fucosyltransferase
MAFKVVQICGGLGNQMFQYAFAKSLQKHLNTPVLLDTTSFDWSNRKMQLELFPIDLPYANAKEIAIAK
MQHLPKLVRDALKYIGFDRVSQEIVFEYEPKLLKPSRLTYFFGYFQDPRYFDAISSLIKQTFTLPPPPEN
NKNNNKKEEEYQRKLSLILAAKNSVFVHIRRGDYVGIGCQLGIDYQKKALEYMAKRVPNMELFVFCED
LKFTQNLDLGYPFTDMTTRDKEEEAYWDMLLMQSCKHGIIANSTYSWWAAYLMENPEKIIIGPKHWLF
GHENILCKEWVKIESHFEVKSQKYNA*
SEQ ID NO: 5 Q9X435-Fucosyltransferase
MAFKVVQICGGLGNQMFQYAFAKSLQKHSNTPVLLDITSFDWSNRKMQLELFPIDLPYASEKEIAIAK
MQHLPKLVRNVLKCMGFDRVSQEIVFEYEPKLLKTSRLTYFYGYFQDPRYFDAISPLIKQTFTLPPPPE
NGNNKKKEEEYHRKLALILAAKNSVFVHIRRGDYVGIGCQLGIDYQKKALEYMAKRVPNMELFVFCED
LEFTQNLDLGYPFMDMTTRDKEEEAYWDMLLMQSCKHGIIANSTYSWWAAYLINNPEKIIIGPKHWLF
GHENILCKEWVKIESHFEVKSQKYNA*
SEQ ID NO: 6 A0A017N3H4-Fucosyltransferase
MKIVQIIGGLGNQMFQFAFYLALKEKYVNVKLDTSSFGAYTHNGFELDKVFHVEYLKASIRERIKLSYQ
GSEIWIRVLRKLLKRKKTEYVEPYLCFDENAISLSCDKYYIGYWQSYKYFTNIEAAIRGQFHFSKVLSDK
NEFIKKQMQNSNSVSLHVRLGDYVNNPAYSNICTSAYYNKAINIIQSKVSEPKFFVFSDDTVWCKDHL
KIPNCHIIDWNNKEESYWDMCLMTYCKHNIIANSSFSWWGAWLNTNPERIVIAPGKWINDDRVQVSDI
IPSDWICV
SEQ ID NO: 7 A0A127VHN4-Fucosyltransferase
MKIIRFLGGLGNQMFQYACYKALSKKYPDVKVDLNSFNFDTAHNGYELEDIFQVSTNKVSPFTGGIYDI
KNRKWIYRKIRRVLNLKKYHKAEEKDFTYDPAIFSNSKSRYYSGFWQNENYFIDIADEIRKDFKFSPLT
AQQNIDTLKKIEQTNSVGVHVRRGDYVNHPAFGGLCEKEYYDQALQIIQSRTEAAKFFLFSNDIDWCV
NNLNIKNCEFISWNKGTQSYIDMQLMSACKHNIIANSSFSWWAAWLNNNPEKIVIGPKKWLHGDQYD
TSALLPAGWIKI
SEQ ID NO: 8 A0A1V6DZU5-Fucosyltransferase
MKIVKIIGGLGNQLFQYAFSRALALKTGDQVLLDITSYNEAQSRIHNGFELPGIFPVHYEVANERDVQR
LSTQPRSALSKVRRKYFTKRTHYIDKIFRFNPQVFDLKGDWYLEGWWQDARYFDFCSDLIRKELTFIA
DPGTENRELAAIFEKSRYSLVPVSLHVRRGDALPNPDTWVCTPIYYRHAIEAARQAVRVLGRMARPY
FLVFSDDLAWCKANLALEPSEAIYVDWNRGANSWRDMWLMSQCRIHVIANSTFSWWGAWLDQHPD
KIVYAPEHWSLAHPRRFAYYHYTFDDVVPAAWKRLPIL
SEQ ID NO: 9 H0XQY2-Fucosyltransferase
MWLARHRHLCLAFLLVCVLSAISFLHFHQDIFRHGLDLSILCPDRHLMTAPVAIFCLEGTPLDPNTSTS
CPQHPASLSGTWTIYPDGRFGNQMGQYATLLALAQLNGRPAFILPAMHATLAPVFRITLPVLSPEVDS
HTPWQELQLHDWMSEEYAHLSDPFLKLSGFPCSWTFFHHLREQIRREFTLHDHLREKAQSLLSQLRL
GLTGDRPRTFVGVHVRRGDYLQVMPQRWRGVVGDQAYLQQAMDWFRARYEAPIFVVTSNGMEWC
RENIDTSQGDVFFAGNGQEGAPGQDFALLTQCNHTIMTIGTFGFWAAYLAGGDTVYLANFTLPDSEF
LKIFKPKAAFLPEWVGINADLS
SEQ ID NO: 10 A0A176TAP2-Fucosyltransferase
MIVVRILGGLGNQMFQYAYARALSLNGYNVKLDLSKIKKYKLHGGYQLDKYNIDLEEADSFSILLGKTG
LKGNKKEKSLLFDNNLKLLNGNEYLKGYFQTEKYFKEIRNTLLTEFVIKQKASKEMLSITKQIEAAKNSC
SLHIRRGDYISNKKANSVHGTCDLEYYKKAIKVISNKYSNITFFVFSDDISWTKENLLIESVTYIDIKSIPH
EDMYLMSLCNHNITANSSFSWWGAWLNKNETKTVIAPKQWYIDKENEIACLNWIKI
SEQ ID NO: 11 A0A1Y0M416-Fucosyltransferase
MITVRIVGGLGNQMFQYAYAKALEQKGYAVKIDTTKFKKYKLHGGYQLDKFNIDLNSTSSLPSILSKIGI
VKSIKEKNLLFDKQLTSLRGNKYVKGYFQTEKYFNEIRAVLLNQFIIKNSISEDTNKVAKTIFSSINSCSL
HIRRGDYISDKKANSVHGTCALEYYEGAIKIMNDTYKNSTFFVFSDDIPWTKENLKIENAFYVDTKTIPH
EDMYLMSLCKNNITANSSFSWWGAWLNKNHTKTVIAPKNWFVNKENEVACENWIKL
SEQ ID NO: 12 C3XIB3-Fucosyltransferase
MGDYKIVELTCGLGNQMFQYAFAKALQKHLQVPVLLDKTWYDTQDNSTQFSLDIFNVDLEYATNTQIE
KAKARVSKLPGLLRKMFGLKKHNIAYSQSFDFHDEYLLPNDFTYFSGFFQNAKYLKGLEQELKSIFYY
DSNNFSNFGKQRLELILQAKNSIFIHIRRGDYCKIGWELGMDYYKRAIQYIMDRVEEPKFFIFGATDMS
FTEQFQKNLGLNENNSANLSEKTITQDNQHEDMFLMCYCKHAILANSSYSFWSAYLNNDANNIVIAPT
PWLLDNDNIICDDWIKISSK
SEQ ID NO: 13 Q8DGK1-Fucosyltransferase
MIIVRLCGGLGNQMFQYAAGLAAAHRIGSEVKFDTHWFDATCLHQGLELRRVFGLELPEPSSKDLRK
VLGACVHPAVRRLLSRRLLRALRPKSLVIQPHFHYWTGFEHLTDNVYLEGYWQSERYFSNIADIIRQQ
FRFVEPLDPHNAALMDEMQSGVSVSLHIRRGDYFNNPQMRRVHGVDLSEYYPAAVATMIEKTNAER
FYVFSDDPQWVLEHLKLPVSYTVVDHNRGAASYRDMQLMSACRHHIIANSTFSWWGAWLNPRPDK
VVIAPRHWFNVDVFDTRDLYCPEWIVL
SEQ ID NO: 14 A0A0G0E0Q0-Fucosyltransferase
MIITRLQGGIGNQMFQYALGRALSVKNNVPLGLDLTFLLDRTPIPNFANFTFRNYDLDVFNIEAVIVSKK
DIPFLYRKHNLGIFMRYIDYFRRKLISTPGKEKMNCSFDASILQLGSDAYLEGWWQSYKYFESIEDIIRK
DFTFKNKLPLHIENLNEVIKKENSLCVHVRRGDYVGNFHHEVVGKDYYDRGIERIKSLTNIDKIYVFSDD
VKWCEGNMKFDLPTMFVGEEYAGTKAEGHMALMSACHNFIIPNSSFSWWGAWLADYKDKVVIVPK
QWFVDASINSDDLIPSGWIRI
SEQ ID NO: 15 A0A1C3GQF1-Fucosyltransferase
MIIAHIIGGLGNQMFQYAAARALSVEKNTGLFLDVTSFESYALHQGFELNKIFSADFKTASYSDIQKILG
WQAPAVVRSILHRPRLAWLRKTTLSIEPSFQYWRGVQDLSDNTYLSGYWQSERYFKNIESIIRKDFSF
KLPMDNENSRIANLISNTEAVSLHIRRGDYVNNSAYSACSLDYYHAAISHFTHMNKPPTFFIFSDDINW
VKEHLKIEHPHCYVDHNNGAASYNDMRLMSLCKHNIIANSSFSWWGAWLNANNDKIVITPKSWFNTN
NHIDDLIPPTWISL
SEQ ID NO: 16 A0A221K4F3-Fucosyltransferase
MIFSRLHGRLGNQMFQYAAARALAEHHCTRVVLDDRTALHKDEGSLLRVFDLPDLAQAPLPPAKHER
PLAYAAWRALGLRPRIRRENGLGYDRAFTQWSDDSYLHGYWQSERYFAAIAQDIRSAFAFRTPMSA
QNTEMAARIASGPSVALHVRRGDYVAVNAMALCDQAYYDAALTSVRKRMENDPTVFVFSDDPAWAK
ENLPLPFEKVVVDFNGPDADYEDLRLMSQCQHNIIANSSFSWWAAWLGETPDSIVAGPAQWFADTA
MSNPDILPARWISVDTSG
SEQ ID NO: 17 C6XYA5-Fucosyltransferase
MKIIRFLGGLGNQMFQYAFYKSLQHRFPHVKADLQGYQEYTLHNGFELEHIFNIKVNSVSSFTSDLFY
NKKWLYRKLRRILNLRNTYIEEKKLFSFDPSLLNNPKSAYYWGYWQNFQYFEHIADDLRKDFQFRAPL
SAQNQEVLDQTKLSNSISLHIRRGDYIKDPLLGGLCGPEYYQTAINYITSKVNAARFFIFSDDIDWCIAN
LKLQDCSFISWNKGTSSYIDMQLMSSCKHHIVANSSFSWWAAWLNPNPDKIVIAPEKWTNDKDINVR
MSFPQGWISL
SEQ ID NO: 18 M5SIY0-Fucosyltransferase
MIVTRLIGGLGNQLFQYAFGHSLARSTYQTLLIDDSAFIDYRLHPLAIDHFTISASRLSDADRSRVPGKF
LRTPVGRALDKVSRFVPGYQGVLPVRREKPFGFRESLLARESDLYLDGYWQSEKFFPGLRGSLREE
FQLREQPSETTRRLSAQMKSENSVAIHVRRGDYVTSAKAKQIYRTLDADYYRRCLLDLAAHETDLKLY
LFSNDVPWCESNLDVGIPFTPVQHTDGATAHEDLHLIAQCRHVVIANSTFSWWGAYLGQLHPTRRVY
YPEPWFHPGTLDGSAMGCDDWISEASLEEQSSLKSSRRAA
SEQ ID NO: 19 A0A1D9LK57-Fucosyltransferase
MIVTRLCGGLGNQLFQYAAARMLAQIHKAKIFVDLGWFENIPNKNTSRYYELDHYRLPIEKLVLESSIQ
KKMLEAPFFNLIPIKRLGLKIYREQSYCFDTNFYNALDNSYLRGYWQSHLYFTDIRDILVKELQPVTPPS
PMDVAIMDMIENSENSISIHVRRGDYVSLKSASNTHGTCSLDYYKKSINFFAEHVSDPHFFVFSDDINW
CRENLSFPHKSTFVSHNNAATAFQDLRLMAHCKHNVIANSSFSWWGAWLNSNDHKIVIAPMNWFNQ
ASHDTKDLLPLNWVRL
SEQ ID NO: 20 A0LYU7-Fucosyltransferase
MSNKNPVIVEIMGGLGNQMFQFAVAKLLAEKNSSVLLVDTNFYKEISQNLKDFPRYFSLGIFDISYKMG
TENGMVNFKNLSFKNRVSRKLGLNYPKIFKEKSYRFDADLFNKKTPIYLKGYFQSYKYFIGVESKIRQ
WFEFPYENLGVGNEEIKSKILEKTSVSVHIRRGDYVENKKTKEFHGNCSLEYYKNAITYFLDIVKEFNIV
FFSDDISWVRDEFKDLPNEKVFVTGNLHENSWKDMYLMSLCDHNIIANSSFSWWAAWLNNNSEKNVI
APKKWFADIDQEQKSLDLLPPSWIRM
SEQ ID NO: 21 F1MS89-Fucosyltransferase
MPGPAPDARAWPDSKHPQTPEWKREKSTDRSIRIQHGSCKLDLSVHEKMRLLGRPAMWAPGHRHL
CLIFLLTCVFACVFFLLIHQNLFHSGLDLFLLCPDRSRVRSPVAILCLSGTPMNPNATFTCPRHSASVS
GTWTIDPKGRFGNQMGQYATLLALAQLNGRQAFIQPSMHAVLAPVFRITLPVLAPEVDRHAPWQELE
LHDWMSEEYAHLKEPWLKLTGFPCSWTFFHHLRDQIRSEFTLHEHLRQEAQRSLSGLRFPRTGGRP
STFVGVHVRRGDYLQVMPLHWKGVVGDRAYLQQAMDWFRARHKAPIFVVTSNGMKWCRENIDTSR
GDVIFAGDGQEGAPNKDFALLTQCNHTIMTIGTFGFWAAYLAGGDTIYLANFTLPDSSFLKIFKPEAAF
LPEWVGINADLSPLQ
SEQ ID NO: 22 R5YN07-Fucosyltransferase
MAVSPQESKYSAHVSPDKPLRIVRLGGGLGNQMFQYAFGLAAGDVLWDNTSFLTNHYRSFDLGLYNI
SGDFASNEQIKKCKNEIRFKNILPRSIRKKFNLGKFIYLKTNRVCERQINRYEPELLSKDGDVYYDGVF
QTEKYFKPLRERLLHDFTLTKPLDAANLDMLAKIRAADAVAVHIRRGDYLNPRSPFTYLDKDYFLNAM
DYIGKRVDKPHFFIFSSDTDWVRTNIQTAYPQTIVEINDEKHGYFDLELMRNCRHNIIANSTFSWWGA
WLNTNPDKIVVAPKQWFRPDAAEYSGDIVPNDWIKL
SEQ ID NO: 23 A0A0G0XT03-Fucosyltransferase
MIIVKIEGGLGNQLFQYAFARGISSRLNTEFKIDKSPFDIYYKYHKYALDNFNLKGSLAKDSDFFGFMW
LKKQHKLFTFFYNHLRFRKKLLPFYFREQAFHFDSSVFSKDNTYFEGYWQTEKYFRELESELQEEITL
NKPLSDYSKGILNQIKSSVAISLHVRRGDYVTGSTISNVPLIHGTCSMDYYKSAIAYISERISNPHFFIFS
DDYDWSVENFKSLKYPTVCIKNGADKNYEDLILMASCKHNIIANSSFSWWGAWLNRNKEKIVIAPKKW
FNMPKQGTNTDDIIPDTWIKL
SEQ ID NO: 24 A0A1E3AA34-Fucosyltransferase
MCCRRIKARNEREVISQLFSEEYIFIMIIIEISGGLGNQMFQYALGQKFISMGKKVKYDLSFYNERVQTL
REFELDIFHIDCPVATSNELFYFGKGFSLASRFKQRIGWDKRNVYEEDLDLGYQPQIFGLDNIYLSGY
WQSEKYFENIRQRILELYTFSGKLGYENKRFLDKIENSNSVSLHVRRGDYLNEENVKIYGGICTINYYK
NAIKYISDRFEKPVFFVFTNDLEWVKNELDIPNKVIVDCNSGSLSYWDMYLMSKCKANIVANSSFSWW
GAWLNQHSNRVVVSPRRWFNNHEQTSTLCDDWVRCGG
SEQ ID NO: 25 A4IFH1-Fucosyltransferase
MWAPGHRHLCLIFLLTCVFACVFFLLIHQNLFHSGLDLFLLCPDRSRVRSPVAILCLSGTPMNPNATFT
CPRHSASVSGTWTIDPKGRFGNQMGQYATLLALAQLNGRQAFIQPSMHAVLAPVFRITLPVLAPEVD
RHAPWQELELHDWMSEEYAHLKEPWLKLTGFPCSWTFFHHLRDQIRSEFTLHEHLRQEAQRSLSGL
RFPRTGGRPSTFVGVHVRRGDYLQVMPLHWKGVVGDRAYLQQAMDWFRARHKAPIFVVTSNGMK
WCRENIDTSRGDVIFAGDGQEGAPNKDFALLTQCNHTIMTIGTFGFWAAYLAGGDTIYLANFTLPDSS
FLKIFKPEAAFLPEWVGINADLSPLQ
SEQ ID NO: 26 F3BFP1-Fucosyltransferase
MIKVKAIGGLGNQLFQYATARAIAEKRGDGVVVDMSDFSSYKTHPFCLNKFRCKATYESKPKLINKLL
SNEKIRNLLQKLGFIKKYYFETQLPFNEDVLLNNSINYLTGYFQSEKYFLSIRECLLDELTLIEDLNIAETA
VSKAIKNAKNSISIHIRRGDYVSNEGANKTHGVCDSDYFKKALNYFSERKLLDEHTELFIFSDDIEWCR
NNLSFDYKMNFVDGSSERPEVDMVLMSQCKHQVISNSTFSWWGAWLNKNDEKVVVAPKEWFKSTD
LDSTDIVPNQWIKL
SEQ ID NO: 27 R7XCZ4-Fucosyltransferase
MIVTRVIGGLGNQMFQYAAGRALARRLGVPLKIDSSGFADYPLHNYGLHHFALKAVQAGDREIPSGR
AENRWAKALRRFGLGTELRVFRERGFAVDPEVMKLPDGTYLDGYWQSESYFAEMTQELRRDFQIAT
PPTSENAEWLARIGGDEGAVSIHVRRGDYVTNASANAVHGICSLDYYMRAARYVAENIGVKPTFYVF
SDDPDWVAGNLHLGHETRYVRHNDSARNYEDLRLMSACRHHIIANSTFSWWGAWLNASEKKVVIAP
AQWFRDEKYDTRDLLPPTWTKL
SEQ ID NO: 28 A0A0G1JR48-Fucosyltransferase
MIIVRLKGGLGNQMFQYATGLAVASRRGQELKLDSTGYDDPRVINSDIPRKYALYAFSISGSIAVRDEV
GKARNPYGVFSKAVRFFNQKVLRKYYADYDPAFFKKNNKYIEGYFQSEKNFGNIKEKVVKEFTLKKEF
ESEFFLTEKNKIDRTKSVSVHIRRGDYVYDPKINSVHGVCSREYYERAINLMKSKIEAPAFYFFSDDIE
WVKKEFGGHSDFRYISNPNLKEYEELILMSLCAHNIIANSSFSWWGAYLNQNPNKIVIAPKKWMNMEP
DPHPNIIPEWWMRI
SEQ ID NO: 29 A0A111R1D7-Fucosyltransferase
MVIVKLIGGLGNQMFQYAAAKALALHTKQELRLDLSGFDDYKLRAFDLHHFNINAKPFRQKSKWIRKL
ENKLKLTTYYNEQSFRFNPEVFNIITKNILLQGYFQAEDYFITYRNDILNDFKIVSPLKKQTQNLLVEMSK
TNAVSIHIRRGDFLTHEVHNTSKEEYYREAMIVIENKIEQPTYYVFSDDMDWVKANFKTKYNTVYVDF
NDASTAFEDIKLMSNCQHNIIANSSFSWWSAWLNTNPNKIVIAPKQWFNGEQYDYTDVVPKRWIKL
SEQ ID NO: 30 A5GEL9-Fucosyltransferase
MIIARLQGGLGNQMFQYAVGLHLALTHNVELKIDITMFSDYKWHTYSLRPFNIRESIATEEEIKALTDVK
MDRPYKKIDNFLCRLLRKSQKISATHVKEKHFHYDPDILKLPDNVYLDGYWQSEKYFKEIENIIRQTFIIK
NPQLGRDKELACKILSTESVCLHIRRGNYVTDKTTNSVLGPCDLSYYSNCIKSLAGNNKDPHFFVFSN
DHEWVSKNLKLDYPTIYVDHNNEDKDYEDLRLMSQCKHHIIANSTFSWWSAWLCSNPDKVIYAPQK
WFRVDEYNTKDLLPSNWLIL
SEQ ID NO: 31 F6ZAB4-Fucosyltransferase
MWAPSRRHLCLIFLLVCVLSSIAFLYVHQGLFHDGVDLFALCPSHHLGTPHVAIFCLSGTTMTSNASLS
CPQQPASLTGTWTIHPDGRFGNQMGQYATLLALAQLNGRQAFILPAMHATLAPVFRITLPVLSPQVD
SQTSWLKLQLHDWMSEEYARVEHPVLKLTGFPCSWTFFHHIREQIRSQFTLHDHLRQDAQGFLSQL
RLGRTGGRPSTFVGVHVRRGDYLQVMPQLWKGVVGDRAYLQQAMDWFRARHEAPIFVVTSNGMD
WCRQNIDTSRGDVIFAGNGLEASPGKDFALLTQCNHTIMTIGTFGFWAAYLAGGDTVYLANFTLPDSN
FLKIFKPEAAFLPEWVGINADLSPLRTPAGR
SEQ ID NO: 32 S9XM23-Fucosyltransferase
MWAPSRRHLCLTFLLVCVSAAILFFHIHQDLLHDALDLSALCPDYNLVTSPVAIFCLSGTPINPNASISC
PKHPASSSGTWTIYPDGRFGNQMGQYATLLALAQLNGRQAFIQPAMHAALAPMFRITLPVLAPEVDR
HAPWRELELHDWMSEEYAHLEEPWLKLTGFPCSWTFFHHLREQILREFTLHDYLRQEAQRLLSRLRL
RRTGKRPSTFVGVHVRRGDYLEVMPHRWKGVVGDRSYLQQAMDWFRARHEAPIFVVTSNGMGWC
RKNIDTSQGDVIFAGNGQEDAPGKDFALLVQCNHTIMTIGTFGFWAAYLAGGDTVYLANFTLPNSKFL
KIFKPKAAFLPEWVGINADLSPLHM
SEQ ID NO: 33 A0A0G1VFP6-Fucosyltransferase
MKIHGGLGNQMFQYALGRNLSLIHKVPVKIDYSYLKTENQSGRRFELDGFRIQAVEATENDIRRYGST
FQKMVDRIRPEAKRKKITEQGDGFHSDVLERFDAYFDGHWQNERYFKAHEKTIREDFSLKNRFGPAS
EAMARKIQSEKNPTSVHIRRGDYVSIEKIADTHGTLPVSYYRTACDKILEKLPDARFFVSSDDIDWAKE
NFPREYPATFISAPEITDCEELTLMSLCKHNIIANSTFSWWGAWLNTNPEKIVIAPKLWFVNPNRVPKD
LIPSSWIPLEAY
SEQ ID NO: 34 A0A1J4UCF9-Fucosyltransferase
MIFDKLSGGLGNQMFQYAAGYSLSLNNRIPLNLDLSSFEHKKTGITHRYFLLNKFNIDRNILINHMEKIS
GYRKFLSKFITKFFGENFYYNITFLSSKYLDGYFQSEKYFKNIEDILRKEFTLKNEMSVVARQVESKISN
SINSVSLHIRRGDYVLDNKTNSYHGICDLDYYKKAVEYFKNKLGELNIFVFSDDIVWVKENLRFENLYF
VSSPDIKDYEELILMSRCKHNIIANSSFSWWGAWLNANKNKTVITPKKWFQKYNINQKHIVPKSWIRL
SEQ ID NO: 35 F8EQF5-Fucosyltransferase
MIIVKLSGGLGNQLFQYAFGRHLATVNQKELKLDTSALTKTSDWTNRSYALDAFNIRAQEATPEEIKAL
AGKPNRLLQRVGRKVGITPIQYFQEPHFHFYSSALSIKSSHYLEGYWQSEKYFEAITPILREEFAFTISP
STHAQTIKEKISNGTSVSIHLRRGDYVKTSKANRYLRPLTMDYYQKAIDYINQRVKNPNFFLFSDDIKW
AKSQVTFPPTTHFSTGTSAHEDLWLMTHCRHHIIANSTFSWWGAWLNQQPDKIVIAPQKWFSTERFD
TKDLLPEPWIQL
SEQ ID NO: 36 Q0V8Q6-Fucosyltransferase
MPGPAPDARAWPDSKHPQTPEWKREKSTDRSIRIQHGSCKLDLSVHEKMRLLGRPAMWAPGHRHL
CLIFLLTCVFACVFFLLIHQNLFHSGLDLFLLCPDRSRVRSPVAILCLSGTPMNPNATFTCPRHSASVS
GTWTIDPKGRFGNQMGQYATLLALAQLNGRQAFIQPSMHAVLAPVFRITLPVLAPEVDRHAPWQELE
LHDWMSEEYAHLKEPWLKLTGFPCSWTFFHHLRDQIRSEFTLHEHLRQEAQRSLSGLRFPRTGGRP
STFVGVHVRRGDYLQVMPLHWKGVVGDRAYLQQAMDWFRARHKAPIFVVTSNGMKWCRENIDTSR
GDVIFAGDGQEGAPNKDFALPTQCNHTIMTIGTFGFWAAYLAGGDTIYLANFTLPDSSFLKIFKPEAAF
LPEWVGINADLSPLQ
SEQ ID NO: 37 W5PRG1-Fucosyltransferase
MWSALAAGALHSSPSRLWAATRQELSGLDLFLLCPDRSRVTSPVAILCLSGTPVNTNATFSCPKHPA
SISGTWTIDPKGRFGNQMGQYATLLALAQLNGRQAFIQPSMHAILAPVFRITLPVLAPEVDRHAPWQE
LELHDWMSEEYAHLKEPWLKLTGFPCSWTFFHHLREQIRSEFTLHEHLRQEAQRSLSGLRFPRTGD
RPSTFVGVHVRRGDYLQVMPLHWKGVVGDHAYLQQAMDWFRARHKAPIFVVTSNGMEWCRENIDT
SRGDVIFAGDGQEGAPHKDFALLTQCNHTIMTIGTFGFWAAYLAGGDTVYLANFTLPDSSFLKIFKPE
AAFLPEWVGINADLSPLQGKAES
SEQ ID NO: 38 A0A0P0L737-Fucosyltransferase
MQITKKGQRNMRLIKVTGGLGNQMFIYAFYLRMKKYYPKVRIDLSDMMHYKVHYGYEMHRVFNLPHT
EFCINQPLKKVIEFLFFKKIYERKQAPNSLRAFEKKYFWPLLYFKGFYQSERFFADIKDEVRESFTFDK
NKANSRSLNMLEILDKDENAVSLHIRRGDYLQPKHWATTGSVCQLPYYQNAIAEMSRRVASPSYYIFS
DDIAWVKENLPLQNAVYIDWNTDEDSWQDMMLMSHCKHHIICNSTFSWWGAWLNPNMDKTVIVPSR
WFQHSEAPDIYPTGWIKVPVS
SEQ ID NO: 39 A0A1U7N023-Fucosyltransferase
MKGKCMGVVIVRLSGGLGNQMFQYAIGRKIALVNNVQLKLDISSFEHDLLRMYNLYWFQIKQAFASSE
ELAALKSLRQTKESNPVIIRLRQVMKRFASWKVFREEQLMPFNPNIMTSSDKIYLDGYWQSEKYFLDI
EDVIRREYTCKYEPNAQSKKIAEMIANSHSVSIHVRRGDYVSNPANNQLHGTCSLTYYQQCVEQIAKE
VLHPHFFVFSDHPIWVKENLCLDYPMTFVTHNNHLRDYEDLWLMSHCQHHIIANSSFSWWAAWLNP
NLNKKVFAPKKWFNDPRLDTRDLLPDNWIKV
SEQ ID NO: 40 F8WY73-Fucosyltransferase
MVTVLLSGGLGNQMFQYAAAKSLAIRLNTALSVDLYTFSKKTQATVRPYELGIFNIEDVVETSSLKAKA
VIKARPFIQRHRSFFQRFGVFTDTYAILYQPTFEALTGGVIMSGYFQNESYFKNISELLRKDFSFKYPLI
GENKDVAGQISENQSVAVHIRRGDYLNKNSQSNFAILEKDYYEKAINYISAHVKNPEFYVFSEDFDWIK
DNLNFKEFPVTFIDWNKGKDSYIDMQLMSLCKHNIIANSSFSWWSAWLNNSEERKIVAPERWFVDEQ
KNELLDCFYPQGWIKI
SEQ ID NO: 41 Q47WH3-Fucosyltransferase
MKVVRVCGGFGNQLFQYAFYLAVKHKFNETTKLDIHDMASYELHNGYELERIFNLNENYCSAEEKLA
VQSTKNIFTKLLKEIKKYTPFIPRTYIKEKKHLHFSYQEVDLGTKDTSIYYRGSWQNPQYFNSIASEIREK
LTFPEFTEPKSLALHQEISEHETVAVHIRRGDYLKHKALGGICDLPYYQNAIKEIEGLVEKPLFVIFSDDI
TWCRANINVEKVRFVDWNSGEQSFQDMHLMSLCTHNIIANSSFSWWGAWLNANPNKIVISPNKWIH
YTDSMGIVPSEWIKVETSI
SEQ ID NO: 42 P0AC88-GDP-mannose dehydratase
MSKVALITGVTGQDGSYLAEFLLEKGYEVHGIKRRASSFNTERVDHIYQDPHTCNPKFHLHYGDLSDT
SNLTRILREVQPDEVYNLGAMSHVAVSFESPEYTADVDAMGTLRLLEAIRFLGLEKKTRFYQASTSEL
YGLVQEIPQKETTPFYPRSPYAVAKLYAYWITVNYRESYGMYACNGILFNHESPRRGETFVTRKITRAI
ANIAQGLESCLYLGNMDSLRDWGHAKDYVKMQWMMLQQEQPEDFVIATGVQYSVRQFVEMAAAQL
GIKLRFEGTGVEEKGIVVSVTGHDAPGVKPGDVIIAVDPRYFRPAEVETLLGDPTKAHEKLGWKPEITL
REMVSEMVANDLEAAKKHSLLKSHGYDVAIALES
SEQ ID NO: 43 A8Y0L5-GDP-mannose dehydratase
MEGLEACIGQSHEVMTTPAAELAAFRARKVALITGISGQDGSYLAELLLSKGYKVHGIIRRSSSFNTARI
EHLYSNPMTHNGDSSFSLHYGDMTDSSCLIKLISTIEPTEVYHLAAQSHVKVSFDLPEYTAEVDAVGTL
RLLDAIHACRLTEKVRFYQASTSELYGKVQEIPQSEKTPFYPRSPYAVAKMYGYWIVVNYREAYKMFA
CNGILFNHESPRRGETFVTRKITRSVAKISLGQQESIELGNLSALRDWGHAREYVEAMWRILQHDAPD
DFVIATGKQFSVREFCNLAFAEIGEVLEWEGEGVEEVGKNKDGIVRVKVSPKYYRPTEVETLLGNPEK
AKKTLGWEAKVTVPELVKEMVASDIALMKANPMA
SEQ ID NO: 44 O45583-GDP-mannose dehydratase
MEARNAEGLESCIEKIQEVKLSSFAELKAFRERKVALITGITGQDGSYLAELLLSKGYKVHGIIRRSSSF
NTARIEHLYGNPVTHNGSASFSLHYGDMTDSSCLIKLISTIEPTEIYHLAAQSHVKVSFDLPEYTAEVDA
VGTLRLLDAIHACRLTEKVRFYQASTSELYGKVQEIPQSELTPFYPRSPYAVAKMYGYWIVVNYREAY
KMFACNGILFNHESPRRGETFVTRKITRSVAKISLRQQEHIELGNLSALRDWGHAKEYVEAMWRILQQ
DTPDDFVIATGKQFSVREFCNLAFAEIGEQLVWEGEGVDEVGKNQDGVVRVKVSPKYYRPTEVETLL
GNPAKARKTLGWEPKITVPELVKEMVASDIALMEADPMA
SEQ ID NO: 45 P93031-GDP-mannose dehydratase
MASENNGSRSDSESITAPKADSTVVEPRKIALITGITGQDGSYLTEFLLGKGYEVHGLIRRSSNFNTQR
INHIYIDPHNVNKALMKLHYADLTDASSLRRWIDVIKPDEVYNLAAQSHVAVSFEIPDYTADVVATGALR
LLEAVRSHTIDSGRTVKYYQAGSSEMFGSTPPPQSETTPFHPRSPYAASKCAAHWYTVNYREAYGLF
ACNGILFNHESPRRGENFVTRKITRALGRIKVGLQTKLFLGNLQASRDWGFAGDYVEAMWLMLQQEK
PDDYVVATEEGHTVEEFLDVSFGYLGLNWKDYVEIDQRYFRPAEVDNLQGDASKAKEVLGWKPQVG
FEKLVKMMVDEDLELAKREKVLVDAGYMDAKQQP
SEQ ID NO: 46 O60547-GDP-mannose dehydratase
MAHAPARCPSARGSGDGEMGKPRNVALITGITGQDGSYLAEFLLEKGYEVHGIVRRSSSFNTGRIEH
LYKNPQAHIEGNMKLHYGDLTDSTCLVKIINEVKPTEIYNLGAQSHVKISFDLAEYTADVDGVGTLRLL
DAVKTCGLINSVKFYQASTSELYGKVQEIPQKETTPFYPRSPYGAAKLYAYWIVVNFREAYNLFAVNGI
LFNHESPRRGANFVTRKISRSVAKIYLGQLECFSLGNLDAKRDWGHAKDYVEAMWLMLQNDEPEDF
VIATGEVHSVREFVEKSFLHIGKTIVWEGKNENEVGRCKETGKVHVTVDLKYYRPTEVDFLQGDCTKA
KQKLNWKPRVAFDELVREMVHADVELMRTNPNA
SEQ ID NO: 47 Q18801-GDP-mannose dehydratase
MPTGKSESSDISEVVGNMEISKVEGLEACIGMSHEVSTTPAAELAAFRARKVALITGISGQDGSYLAEL
LLSKGYKVHGIIRRSSSFNTARIEHLYSNPITHHGDSSFSLHYGDMTDSSCLIKLISTIEPTEVYHLAAQS
HVKVSFDLPEYTAEVDAVGTLRLLDAIHACRLTEKVRFYQASTSELYGKVQEIPQSEKTPFYPRSPYA
VAKMYGYWIVVNYREAYNMFACNGILFNHESPRRGETFVTRKITRSVAKISLGQQESIELGNLSALRD
WGHAREYVEAMWRILQHDSPDDFVIATGKQFSVREFCNLAFAEIGEVLQWEGEGVEEVGKNKDGVI
RVKVSPKYYRPTEVETLLGNAEKAKKTLGWEAKVTVPELVKEMVASDIILMKSNPMA
SEQ ID NO: 48 Q8K3X3-GDP-mannose dehydratase
MAHAPASCPSSRNSGDGDKGKPRKVALITGITGQDGSYLAEFLLEKGYEVHGIVRRSSSFNTGRIEHL
YKNPQAHIEGNMKLHYGDLTDSTCLVKIINEVKPTEIYNLGAQSHVKISFDLAEYTADVDGVGTLRLLD
AIKTCGLINSVKFYQASTSELYGKVQEIPQKETTPFYPRSPYGAAKLYAYWIVVNFREAYNLFAVNGILF
NHESPRRGANFVTRKISRSVAKIYLGQLECFSLGNLDAKRDWGHAKDYVEAMWLMLQNDEPEDFVIA
TGEVHSVREFVEKSFMHIGKTIVWEGKNENEVGRCKETGKIHVTVDLKYYRPTEVDFLQGDCSKAQQ
KLNWKPRVAFDELVREMVQADVELMRTNPNA
SEQ ID NO: 49 Q1ZXF7-GDP-mannose dehydratase
MSEERKVALITGITGQDGSYLTEFLISKGYYVHGIIQKIFHHFNTIVKNIYIKIDMLKEKESLTLHYGDLTD
ASNLHSIVSKVNPTEIYNLGAQSHVKVSFDMSEYTGDVDGLGCLRLLDAIRSCGMEKKVKYYQASTSE
LYGKVQEIPQSETTPFYPRSPYAVAKQYAYWIVVNYREAYDMYACNGILFNHESPRRGPTFVTRKITR
FVAGIACGRDEILYLGNINAKRDWGHARDYVEAMWLMLQQEKPEDFVIATGETHSVREFVEKSFKEID
IIIKWRGEAEKEEGYCEKTGKVYVKIDEKYYRPTEVDLLLGNPNKAKKLLQWQIKTSFGELVKEMVAKD
IEYIKNGDKYN
SEQ ID NO: 50 P55354-GDP-mannose dehydratase
MTDRKVALISGVTGQDGAYLAELLLDEGYIVHGIKRRSSSFNTQRIEHIYQERHDPEARFFLHYGDMT
DSTNLLRIVQQTQPHEIYNLAAQSHVQVSFETPEYTANADAIGTLRMLEAIRILGLTNRTRFYQASTSEL
YGLAQESPQNEKTPFYPRSPYAAAKLYAYWIVVNYREAYGMHASNGILFNHESPLRGETFVTRKITRA
AAAISLGKQEVLYLGNLDAQRDWGHAREYVRGMWMMCQQDRPGDYVLATGVTTSVRTFVEWAFE
ETGMTIEWVGEGIEERGIDAATGRCVVAVDPRYFRPTEVDLLLGDATKARQVLGWRHETSVRDLACE
MVREDLSYLRGTRQ
SEQ ID NO: 51 Q9SNY3-GDP-mannose dehydratase
MASRSLNGDSDIVKPRKIALVTGITGQDGSYLTEFLLEKGYEVHGLIRRSSNFNTQRLNHIYVDPHNVN
KALMKLHYGDLSDASSLRRWLDVIKPDEVYNLAAQSHVAVSFEIPDYTADVVATGALRLLEAVRSHNI
DNGRAIKYYQAGSSEMFGSTPPPQSETTPFHPRSPYAASKCAAHWYTVNYREAYGLYACNGILFNH
ESPRRGENFVTRKITRALGRIKVGLQTKLFLGNIQASRDWGFAGDYVEAMWLMLQQEKPDDYVVATE
ESHTVKEFLDVSFGYVGLNWKDHVEIDKRYFRPTEVDNLKGDASKAKEMLGWKPKVGFEKLVKMMV
DEDLELAKREKVLADAGYMDAQQQP
SEQ ID NO: 52 O85713-GDP-mannose dehydratase
MTDRKVALISGVTGQDGAYLAELLLDEGYIVHGIKRRSSSFNTQRIEHIYQERHDPEARFFLHYGDMT
DSTNLLRIVQQTQPHEIYNLAAQSHVQVSFETPEYTANADAIGTLRMLEAIRILGLIHRTRFYQASTSEL
YGLAQEIPQNEKTPFYPRSPYAAAKLYAYWIVVNYREAYGMHASNGILFNHESPLRGETFVTRKITRA
AAAISLGKQEVLYLGNLDAQRDWGHAREYVRGMWMMCQQDRPGDYVLATGVTTSVRTFVEWAFE
ETGMTIEWVGEGIEERGIDAATGKCVVAVDPRYFRPTEVDLLLGDATKARQVLGWRHETSVRDLACE
MVREDLSYLRGTRQ
SEQ ID NO: 53 Q8K0C9-GDP-mannose dehydratase
MAQAPAKCPSYPGSGDGEMGKLRKVALITGITGQDGSYLAEFLLEKGYEVHGIVRRSSSFNTGRIEHL
YKNPQAHIEGNMKLHYGDLTDSTCLVKIINEVKPTEIYNLGAQSHVKISFDLAEYTADVDGVGTLRLLD
AIKTCGLINSVKFYQASTSELYGKVQEIPQKETTPFYPRSPYGAAKLYAYWIVVNFREAYNLFAVNGILF
NHESPRRGANFVTRKISRSVAKIYLGQLECFSLGNLDAKRDWGHAKDYVEAMWLMLQNDEPEDFVIA
TGEVHSVREFVEKSFMHIGKTIVWEGKNENEVGRCKETGKVHVTVDLKYYRPTEVDFLQGDCSKAQ
QKLNWKPRVAFDELVREMVQADVELMRTNPNA
SEQ ID NO: 54 Q9SNY3-GDP-mannose dehydratase
MASRSLNGDSDIVKPRKIALVTGITGQDGSYLTEFLLEKGYEVHGLIRRSSNFNTQRLNHIYVDPHNVN
KALMKLHYGDLSDASSLRRWLDVIKPDEVYNLAAQSHVAVSFEIPDYTADVVATGALRLLEAVRSHNI
DNGRAIKYYQAGSSEMFGSTPPPQSETTPFHPRSPYAASKCAAHWYTVNYREAYGLYACNGILFNH
ESPRRGENFVTRKITRALGRIKVGLQTKLFLGNIQASRDWGFAGDYVEAMWLMLQQEKPDDYVVATE
ESHTVKEFLDVSFGYVGLNWKDHVEIDKRYFRPTEVDNLKGDASKAKEMLGWKPKVGFEKLVKMMV
DEDLELAKREKVLADAGYMDAQQQP
SEQ ID NO: 55 Q18801-GDP-mannose dehydratase
MPTGKSESSDISEVVGNMEISKVEGLEACIGMSHEVSTTPAAELAAFRARKVALITGISGQDGSYLAEL
LLSKGYKVHGIIRRSSSFNTARIEHLYSNPITHHGDSSFSLHYGDMTDSSCLIKLISTIEPTEVYHLAAQS
HVKVSFDLPEYTAEVDAVGTLRLLDAIHACRLTEKVRFYQASTSELYGKVQEIPQSEKTPFYPRSPYA
VAKMYGYWIVVNYREAYNMFACNGILFNHESPRRGETFVTRKITRSVAKISLGQQESIELGNLSALRD
WGHAREYVEAMWRILQHDSPDDFVIATGKQFSVREFCNLAFAEIGEVLQWEGEGVEEVGKNKDGVI
RVKVSPKYYRPTEVETLLGNAEKAKKTLGWEAKVTVPELVKEMVASDIILMKSNPMA
SEQ ID NO: 56 Q1ZXF7-GDP-mannose dehydratase
MSEERKVALITGITGQDGSYLTEFLISKGYYVHGIIQKIFHHFNTIVKNIYIKIDMLKEKESLTLHYGDLTD
ASNLHSIVSKVNPTEIYNLGAQSHVKVSFDMSEYTGDVDGLGCLRLLDAIRSCGMEKKVKYYQASTSE
LYGKVQEIPQSETTPFYPRSPYAVAKQYAYWIVVNYREAYDMYACNGILFNHESPRRGPTFVTRKITR
FVAGIACGRDEILYLGNINAKRDWGHARDYVEAMWLMLQQEKPEDFVIATGETHSVREFVEKSFKEID
IIIKWRGEAEKEEGYCEKTGKVYVKIDEKYYRPTEVDLLLGNPNKAKKLLQWQIKTSFGELVKEMVAKD
IEYIKNGDKYN
SEQ ID NO: 57 Q9VMW9-GDP-mannose dehydratase
MLNTRLIAMSTSDGAPETKKQRPESSSNGSKDQNGTEAGAEGDSRDKVALITGITGQDGSYLAEFLL
KKDYEVHGIIRRASTFNTTRIEHLYADPKAHKGGRMKLHYGDMTDSSSLVKIINMVKPTEIYNLAAQSH
VKVSFDLSEYTAEVDAVGTLRILDAIRTCGMEKNVRFYQASTSELYGKVVETPQNEQTPFYPRSPYAC
AKMYGFWIVINYREAYNMYACNGILFNHESPRRGENFVTRKITRSVAKIYHKQMEYFELGNLDSKRD
WGHASDYVEAMWMMLQRESPSDYVIATGETHSVREFVEAAFKHIDREITWKGKGVDEVGVENGTGI
VRVRINPKYFRPTEVDLLQGDASKANRELNWTPKVTFVELVSDMMKADIELMRKNPIA
SEQ ID NO: 58 Q9VMW9-GDP-mannose dehydratase
MLNTRLIAMSTSDGAPETKKQRPESSSNGSKDQNGTEAGAEGDSRDKVALITGITGQDGSYLAEFLL
KKDYEVHGIIRRASTFNTTRIEHLYADPKAHKGGRMKLHYGDMTDSSSLVKIINMVKPTEIYNLAAQSH
VKVSFDLSEYTAEVDAVGTLRILDAIRTCGMEKNVRFYQASTSELYGKVVETPQNEQTPFYPRSPYAC
AKMYGFWIVINYREAYNMYACNGILFNHESPRRGENFVTRKITRSVAKIYHKQMEYFELGNLDSKRD
WGHASDYVEAMWMMLQRESPSDYVIATGETHSVREFVEAAFKHIDREITWKGKGVDEVGVENGTGI
VRVRINPKYFRPTEVDLLQGDASKANRELNWTPKVTFVELVSDMMKADIELMRKNPIA
SEQ ID NO: 59 045583-GDP-mannose dehydratase
MEARNAEGLESCIEKIQEVKLSSFAELKAFRERKVALITGITGQDGSYLAELLLSKGYKVHGIIRRSSSF
NTARIEHLYGNPVTHNGSASFSLHYGDMTDSSCLIKLISTIEPTEIYHLAAQSHVKVSFDLPEYTAEVDA
VGTLRLLDAIHACRLTEKVRFYQASTSELYGKVQEIPQSELTPFYPRSPYAVAKMYGYWIVVNYREAY
KMFACNGILFNHESPRRGETFVTRKITRSVAKISLRQQEHIELGNLSALRDWGHAKEYVEAMWRILQQ
DTPDDFVIATGKQFSVREFCNLAFAEIGEQLVWEGEGVDEVGKNQDGVVRVKVSPKYYRPTEVETLL
GNPAKARKTLGWEPKITVPELVKEMVASDIALMEADPMA
SEQ ID NO: 60 A8Y0L5-GDP-mannose dehydratase
MEGLEACIGQSHEVMTTPAAELAAFRARKVALITGISGQDGSYLAELLLSKGYKVHGIIRRSSSFNTARI
EHLYSNPMTHNGDSSFSLHYGDMTDSSCLIKLISTIEPTEVYHLAAQSHVKVSFDLPEYTAEVDAVGTL
RLLDAIHACRLTEKVRFYQASTSELYGKVQEIPQSEKTPFYPRSPYAVAKMYGYWIVVNYREAYKMFA
CNGILFNHESPRRGETFVTRKITRSVAKISLGQQESIELGNLSALRDWGHAREYVEAMWRILQHDAPD
DFVIATGKQFSVREFCNLAFAEIGEVLEWEGEGVEEVGKNKDGIVRVKVSPKYYRPTEVETLLGNPEK
AKKTLGWEAKVTVPELVKEMVASDIALMKANPMA
SEQ ID NO: 61 Q8K0C9-GDP-mannose dehydratase
MAQAPAKCPSYPGSGDGEMGKLRKVALITGITGQDGSYLAEFLLEKGYEVHGIVRRSSSFNTGRIEHL
YKNPQAHIEGNMKLHYGDLTDSTCLVKIINEVKPTEIYNLGAQSHVKISFDLAEYTADVDGVGTLRLLD
AIKTCGLINSVKFYQASTSELYGKVQEIPQKETTPFYPRSPYGAAKLYAYWIVVNFREAYNLFAVNGILF
NHESPRRGANFVTRKISRSVAKIYLGQLECFSLGNLDAKRDWGHAKDYVEAMWLMLQNDEPEDFVIA
TGEVHSVREFVEKSFMHIGKTIVWEGKNENEVGRCKETGKVHVTVDLKYYRPTEVDFLQGDCSKAQ
QKLNWKPRVAFDELVREMVQADVELMRTNPNA
SEQ ID NO: 62 Q8K3X3-GDP-mannose dehydratase
MAHAPASCPSSRNSGDGDKGKPRKVALITGITGQDGSYLAEFLLEKGYEVHGIVRRSSSFNTGRIEHL
YKNPQAHIEGNMKLHYGDLTDSTCLVKIINEVKPTEIYNLGAQSHVKISFDLAEYTADVDGVGTLRLLD
AIKTCGLINSVKFYQASTSELYGKVQEIPQKETTPFYPRSPYGAAKLYAYWIVVNFREAYNLFAVNGILF
NHESPRRGANFVTRKISRSVAKIYLGQLECFSLGNLDAKRDWGHAKDYVEAMWLMLQNDEPEDFVIA
TGEVHSVREFVEKSFMHIGKTIVWEGKNENEVGRCKETGKIHVTVDLKYYRPTEVDFLQGDCSKAQQ
KLNWKPRVAFDELVREMVQADVELMRTNPNA
SEQ ID NO: 63 O85713-GDP-mannose dehydratase
MTDRKVALISGVTGQDGAYLAELLLDEGYIVHGIKRRSSSFNTQRIEHIYQERHDPEARFFLHYGDMT
DSTNLLRIVQQTQPHEIYNLAAQSHVQVSFETPEYTANADAIGTLRMLEAIRILGLIHRTRFYQASTSEL
YGLAQEIPQNEKTPFYPRSPYAAAKLYAYWIVVNYREAYGMHASNGILFNHESPLRGETFVTRKITRA
AAAISLGKQEVLYLGNLDAQRDWGHAREYVRGMWMMCQQDRPGDYVLATGVTTSVRTFVEWAFE
ETGMTIEWVGEGIEERGIDAATGKCVVAVDPRYFRPTEVDLLLGDATKARQVLGWRHETSVRDLACE
MVREDLSYLRGTRQ
SEQ ID NO: 64 P55354-GDP-mannose dehydratase
MTDRKVALISGVTGQDGAYLAELLLDEGYIVHGIKRRSSSFNTQRIEHIYQERHDPEARFFLHYGDMT
DSTNLLRIVQQTQPHEIYNLAAQSHVQVSFETPEYTANADAIGTLRMLEAIRILGLTNRTRFYQASTSEL
YGLAQESPQNEKTPFYPRSPYAAAKLYAYWIVVNYREAYGMHASNGILFNHESPLRGETFVTRKITRA
AAAISLGKQEVLYLGNLDAQRDWGHAREYVRGMWMMCQQDRPGDYVLATGVTTSVRTFVEWAFE
ETGMTIEWVGEGIEERGIDAATGRCVVAVDPRYFRPTEVDLLLGDATKARQVLGWRHETSVRDLACE
MVREDLSYLRGTRQ
SEQ ID NO: 65 P07921-lactose permease
MADHSSSSSSLQKKPINTIEHKDTLGNDRDHKEALNSDNDNTSGLKINGVPIEDAREEVLLPGYLSKQ
YYKLYGLCFITYLCATMQGYDGALMGSIYTEDAYLKYYHLDINSSSGTGLVFSIFNVGQICGAFFVPLM
DWKGRKPAILIGCLGVVIGAIISSLTTTKSALIGGRWFVAFFATIANAAAPTYCAEVAPAHLRGKVAGLY
NTLWSVGSIVAAFSTYGTNKNFPNSSKAFKIPLYLQMMFPGLVCIFGWLIPESPRWLVGVGREEEARE
FIIKYHLNGDRTHPLLDMEMAEIIESFHGTDLSNPLEMLDVRSLFRTRSDRYRAMLVILMAWFGQFSG
NNVCSYYLPTMLRNVGMKSVSLNVLMNGVYSIVTWISSICGAFFIDKIGRREGFLGSISGAALALTGLSI
CTARYEKTKKKSASNGALVFIYLFGGIFSFAFTPMQSMYSTEVSTNLTRSKAQLLNFVVSGVAQFVNQ
FATPKAMKNIKYWFYVFYVFFDIFEFIVIYFFFVETKGRSLEELEVVFEAPNPRKASVDQAFLAQVRATL
VQRNDVRVANAQNLKEQEPLKSDADHVEKLSEAESV*
SEQ ID NO: 66 Q7SCU1-lactose permease
MSSHGSHDGASTEKHLATHDIAPTHDAIKIVPKGHGQTATKPGAQEKEVRNAALFAAIKESNIKPWSK
ESIHLYFAIFVAFCCACANGYDGSLMTGIIAMDKFQNQFHTGDTGPKVSVIFSLYTVGAMVGAPFAAIL
SDRFGRKKGMFIGGIFIIVGSIIVASSSKLAQFVVGRFVLGLGIAIMTVAAPAYSIEIAPPHWRGRCTGFY
NCGWFGGSIPAACITYGCYFIKSNWSWRIPLILQAFTCLIVMSSVFFLPESPRFLFANGRDAEAVAFLV
KYHGNGDPNSKLVLLETEEMRDGIRTDGVDKVWWDYRPLFMTHSGRWRMAQVLMISIFGQFSGNG
LGYFNTVIFKNIGVTSTSQQLAYNILNSVISAIGALTAVSMTDRMPRRAVLIIGTFMCAAALATNSGLSAT
LDKQTQRGTQINLNQGMNEQDAKDNAYLHVDSNYAKGALAAYFLFNVIFSFTYTPLQGVIPTEALETTI
RGKGLALSGFIVNAMGFINQFAGPIALHNIGYKYIFVFVGWDLIETVAWYFFGVESQGRTLEQLEWVY
DQPNPVKASLKVEKVVVQADGHVSEAIVA*
SEQ ID NO: 67 OR74A-lactose permease
MSSHGSHDGASTEKHLATHDIAPTHDAIKIVPKGHGQTATKPGAQEKEVRNAALFAAIKESNIKPWSK
ESIHLYFAIFVAFCCACANGYDGSLMTGIIAMDKFQNQFHTGDTGPKVSVIFSLYTVGAMVGAPFAAIL
SDRFGRKKGMFIGGIFIIVGSIIVASSSKLAQFVVGRFVLGLGIAIMTVAAPAYSIEIAPPHWRGRCTGFY
NCGWFGGSIPAACITYGCYFIKSNWSWRIPLILQAFTCLIVMSSVFFLPESPRFLFANGRDAEAVAFLV
KYHGNGDPNSKLVLLETEEMRDGIRTDGVDKVWWDYRPLFMTHSGRWRMAQVLMISIFGQFSGNG
LGYFNTVIFKNIGVTSTSQQLAYNILNSVISAIGALTAVSMTDRMPRRAVLIIGTFMCAAALATNSGLSAT
LDKQTQRGTQINLNQGMNEQDAKDNAYLHVDSNYAKGALAAYFLFNVIFSFTYTPLQGVIPTEALETTI
RGKGLALSGFIVNAMGFINQFAGPIALHNIGYKYIFVFVGWDLIETVAWYFFGVESQGRTLEQLEWVY
DQPNPVKASLKVEKVVVQADGHVSEAIVA*
SEQ ID NO: 68 Q7SD12-lactose permease
MGIFNKKPVAQAVDLNQIQEEAPQFERVDWKKDPGLRKLYFYAFILCIASATTGYDGMFFNSVQNFET
WIKYFGDPRGSELGLLGALYQIGSIGSIPFVPLLTDNFGRKTPIIIGCVIMIVGAVLQATAKNLDTFMGGR
TMLGFGNSLAQIASPMLLTELAHPQHRARLTTIYNCLWNVGALVVSWLAFGTNYINNDWSWRIPALLQ
AFPSIIQLLGIWWVPESPRFLIAKDKHDEALHILAKYHANGDPNHPTVQFEFREIKETIRLEMESTKNSS
YLDFFKSRGNRYRLAILLSLGFFSQWSGNAIISNYSSKLYETAGVTDSTAKLGLSAGQTGLALIVSVTM
ALLVDKLGRRLAFLASTGGMCGTFVIWTLTAGLYGEHRLKGADKAMIFFIWVFGIFYSLAWSGLLVGY
AIEILPYRLRGKGLMVMNMSVQCALTLNTYANPVAFDYFGPDHSWKLYLIYTCWIAAEFVFVFFMYVE
TKGPTLEELAKVIDGDEADVAHIDIHQVEKEVEIHEHEGKSVA*
SEQ ID NO: 69 OR74A-lactose permease
MGIFNKKPVAQAVDLNQIQEEAPQFERVDWKKDPGLRKLYFYAFILCIASATTGYDGMFFNSVQNFET
WIKYFGDPRGSELGLLGALYQIGSIGSIPFVPLLTDNFGRKTPIIIGCVIMIVGAVLQATAKNLDTFMGGR
TMLGFGNSLAQIASPMLLTELAHPQHRARLTTIYNCLWNVGALVVSWLAFGTNYINNDWSWRIPALLQ
AFPSIIQLLGIWWVPESPRFLIAKDKHDEALHILAKYHANGDPNHPTVQFEFREIKETIRLEMESTKNSS
YLDFFKSRGNRYRLAILLSLGFFSQWSGNAIISNYSSKLYETAGVTDSTAKLGLSAGQTGLALIVSVTM
ALLVDKLGRRLAFLASTGGMCGTFVIWTLTAGLYGEHRLKGADKAMIFFIWVFGIFYSLAWSGLLVGY
AIEILPYRLRGKGLMVMNMSVQCALTLNTYANPVAFDYFGPDHSWKLYLIYTCWIAAEFVFVFFMYVE
TKGPTLEELAKVIDGDEADVAHIDIHQVEKEVEIHEHEGKSVA*
SEQ ID NO: 70 C8VN19-lactose permease
MERRTYGFETTISRDADKGVFSVNNAALHMATLRVKPRLLTKRMLKLYWCIGVAMLNSCINGYNGSL
MGSINSYRQYREYFGFDLEEGTSTTGIVYAIYTIGNIVGSFFAGPFTDFRGRRMGMAIGALWIIAGTIVQ
ATCHNLGGFMAGRELLGFGVATSATAGPAYVSEMAHPAYRGAMTGLYNVLWFGGGIPGTFIPWRTS
TIDGTQSWRIPVWLQMVFSGLVLLLCFTIPESPRWLISCDRHEAAIRVLAEYHGEGDRNSPLVQLEYR
EMLEDISNVGADKRWWDYRELFDSRETRYRSMLVVFMAFFGQWSGNGPVSYYYPQMLAGAGISSN
HTRLLLQGLQNIVQFTGAIFGALITDRVGRRPQLLVSTSIIVFLFVIITALNATNVQVAGDGGGVVAKSSV
TARAQIAMIFIFGFVYSAGWTPNQAMYPVECLRYESRAKGMGMNNFFINIASFYNTFVTGIAFTRIGWK
YYFLFIFWCTFEVLIIYFLFVETSKRTLEELTVIFQQKRPVQASLDKEEIFVSGDEIVEVRSW*
SEQ ID NO: 71 FGSC_A4-lactose permease
MERRTYGFETTISRDADKGVFSVNNAALHMATLRVKPRLLTKRMLKLYWCIGVAMLNSCINGYNGSL
MGSINSYRQYREYFGFDLEEGTSTTGIVYAIYTIGNIVGSFFAGPFTDFRGRRMGMAIGALWIIAGTIVQ
ATCHNLGGFMAGRFLLGFGVATSATAGPAYVSEMAHPAYRGAMTGLYNVLWFGGGIPGTFIPWRTS
TIDGTQSWRIPVWLQMVFSGLVLLLCFTIPESPRWLISCDRHEAAIRVLAEYHGEGDRNSPLVQLEYR
EMLEDISNVGADKRWWDYRELFDSRETRYRSMLVVFMAFFGQWSGNGPVSYYYPQMLAGAGISSN
HTRLLLQGLQNIVQFTGAIFGALITDRVGRRPQLLVSTSIIVFLFVIITALNATNVQVAGDGGGVVAKSSV
TARAQIAMIFIFGFVYSAGWTPNQAMYPVECLRYESRAKGMGMNNFFINIASFYNTFVTGIAFTRIGWK
YYFLFIFWCTFEVLIIYFLFVETSKRTLEELTVIFQQKRPVQASLDKEEIFVSGDEIVEVRSW*
SEQ ID NO: 72 R1EG36-lactose permease
MDTKDVALHREQSLDEKANVTAVKEITGNEAFNEALLKEPPQPFNGASIVLYLCCLVGFFCSTMNGYD
GSLLNGLLMSDDFKAYFGGSDKGIWAGIVTAMYQIGSVVALPFVGPAIDNFGRKGGMFIGALIIVIGTVI
NATTMFTASIGQFEAGRFILGFGVSIATAAGPMYVVEVTHPAYRGVMTALYNTFWFTGSILAAGAVRG
SLDSTLKHNWVIPVWLQLLFSGLIVLFVYFLPESPRWLYVNNKREQCRDILTKYHGNGNENSIWVSLQ
LREYEEYLEMDGADKRWWDYRALFRDRASRYRIACNIVISIFGQWAGNAVLTYFMSALLESAGYTTE
VSKANINLFYSCEQFLIAVAGALCVDKIGRRPLLLGAMLGCSLVWVGMTIATSQFDKTGSTDASKAAT
AMIFLFGAVFSFGVTPLQALYPVEVLSFEMRAKGMAFSSFALNAAMLLNQFAWPVSMEKIGWRTYIIF
VIWDSIQAGVIYFFIPETKNRTLEELDDIFHAKNPTKASLQKRKVALDANANVVGVEPLDSDSA*
SEQ ID NO: 73 R1EG36-lactose permease
MDTKDVALHREQSLDEKANVTAVKEITGNEAFNEALLKEPPQPFNGASIVLYLCCLVGFFCSTMNGYD
GSLLNGLLMSDDFKAYFGGSDKGIWAGIVTAMYQIGSVVALPFVGPAIDNFGRKGGMFIGALIIVIGTVI
NATTMFTASIGQFEAGRFILGFGVSIATAAGPMYVVEVTHPAYRGVMTALYNTFWFTGSILAAGAVRG
SLDSTLKHNWVIPVWLQLLFSGLIVLFVYFLPESPRWLYVNNKREQCRDILTKYHGNGNENSIWVSLQ
LREYEEYLEMDGADKRWWDYRALFRDRASRYRIACNIVISIFGQWAGNAVLTYFMSALLESAGYTTE
VSKANINLFYSCEQFLIAVAGALCVDKIGRRPLLLGAMLGCSLVWVGMTIATSQFDKTGSTDASKAAT
AMIFLFGAVFSFGVTPLQALYPVEVLSFEMRAKGMAFSSFALNAAMLLNQFAWPVSMEKIGWRTYIIF
VIWDSIQAGVIYFFIPETKNRTLEELDDIFHAKNPTKASLQKRKVALDANANVVGVEPLDSDSA*
SEQ ID NO: 74 V2XMN5-lactose permease
MSKESPVPSFSNDIKGSIEHVEDTIPSLEGQRKKRFSGPEERQAALEEAIKRDPGPGQWSWPTIHMCI
ITFIICCCSGDSGFDSTVMGGINGMHQFQEYFGMTGAGTKTSIVFGIYTIGQLCGTIPAAYFPDRFGRR
FSMFFGNCILICGAVITANAKSMSMFLGGRWMTGFGCTCAATSAKSYLAEIVPPRTRGAYLGFLNSFY
YVGQMSASGMMVATNLWPNELSWRLPLYIQTVPAAINALFVFTCPESPRWLASIGKHEQARKLLAKF
HSQDGNINSPVIEIEMDEIKEKIEINGRDKRWWDFRPLFRTRSDRYRSYMCIIIGAFGQLSGNGLITYFL
PILIKNAGIQSQSKQQTLNFINSVTSYIGALAGSFTVDRFGRRKNLFWATFTITCILAIVTGLLSQNGNAT
RSNAGISFIFLFMVCFSFGWTPMQALYPAEVLSYEARAKGLAFLNLVTQAASLINTFGLPVALEKIGWK
TYVIFVAWDAFECVIIYFFIVETKYLTLEEIGEVFEQPNPVQYSKELYRRRATGGNDEEAH*
SEQ ID NO: 75 V2XMN5-lactose permease
MSKESPVPSFSNDIKGSIEHVEDTIPSLEGQRKKRFSGPEERQAALEEAIKRDPGPGQWSWPTIHMCI
ITFIICCCSGDSGFDSTVMGGINGMHQFQEYFGMTGAGTKTSIVFGIYTIGQLCGTIPAAYFPDRFGRR
FSMFFGNCILICGAVITANAKSMSMFLGGRWMTGFGCTCAATSAKSYLAEIVPPRTRGAYLGFLNSFY
YVGQMSASGMMVATNLWPNELSWRLPLYIQTVPAAINALFVFTCPESPRWLASIGKHEQARKLLAKF
HSQDGNINSPVIEIEMDEIKEKIEINGRDKRWWDFRPLFRTRSDRYRSYMCIIIGAFGQLSGNGLITYFL
PILIKNAGIQSQSKQQTLNFINSVTSYIGALAGSFTVDRFGRRKNLFWATFTITCILAIVTGLLSQNGNAT
RSNAGISFIFLFMVCFSFGWTPMQALYPAEVLSYEARAKGLAFLNLVTQAASLINTFGLPVALEKIGWK
TYVIFVAWDAFECVIIYFFIVETKYLTLEEIGEVFEQPNPVQYSKELYRRRATGGNDEEAH*
SEQ ID NO: 76 A0A194XU26-lactose permease
MFAVLTSATNGYDGSMMNGLQALPQWEASFHNPGPSTRGLLNAIMSVGSIVALPITPYIADILGRRAG
VMTGCVIMIIGVVLQSIGINIQMFIAARFLIGFGVAIAHGSAPLLIAELVHPQHRAIFTTIYNSTWYFGSIVA
SWLTYGTFQLAGPWAWRIPSIVQAAPSCLQLIAIWMVPESPRYLIAKGKNEKALNILAKAHANGNVED
ELVQIEYREIRETLQLEKEFEQNGWLEFFQTKGNRHRLIILISLGFFSQWSGNGLVSYYMNQVLQGAG
VTSAKLRLEINGILNIINFLTAVTMCFFIDKFGRRPLFLFATAGMCASFCIWTICAAEFTKTAVAAAGQAE
VAFIFIYYVFYNCAWSGLLVGYAVEILPYKLRAKGLTLMFLAVDLALFFNSYVNPVALAALDWKYYIVYD
VWLFVELCVVFFFYIETRNTPLEEIVKYFDGEQALLGGDMATEKARAILIEEAGDHHNKTAIAHTEEVSS
PNSQDGKI*
SEQ ID NO: 77A0A194XU26-lactose permease
MFAVLTSATNGYDGSMMNGLQALPQWEASFHNPGPSTRGLLNAIMSVGSIVALPITPYIADILGRRAG
VMTGCVIMIIGVVLQSIGINIQMFIAARFLIGFGVAIAHGSAPLLIAELVHPQHRAIFTTIYNSTWYFGSIVA
SWLTYGTFQLAGPWAWRIPSIVQAAPSCLQLIAIWMVPESPRYLIAKGKNEKALNILAKAHANGNVED
ELVQIEYREIRETLQLEKEFEQNGWLEFFQTKGNRHRLIILISLGFFSQWSGNGLVSYYMNQVLQGAG
VTSAKLRLEINGILNIINFLTAVTMCFFIDKFGRRPLFLFATAGMCASFCIWTICAAEFTKTAVAAAGQAE
VAFIFIYYVFYNCAWSGLLVGYAVEILPYKLRAKGLTLMFLAVDLALFFNSYVNPVALAALDWKYYIVYD
VWLFVELCVVFFFYIETRNTPLEEIVKYFDGEQALLGGDMATEKARAILIEEAGDHHNKTAIAHTEEVSS
PNSQDGKI*
SEQ ID NO: 78 A0A1S8B7R5-lactose permease
MFKDLGGHPTLTTALVISVCVVDSVTVAYDGSLMGSLNAMPAYSDYFTLTTATTSLNTASTFIGAILLS
PFAALLINWRGRKCGIYVSALVQIAGTILQGAAQSIGMFIVGRLLIGAGSGLAQTSAATYVAETVPSKIR
ALALGLYFTCWAVGALLAAGVCYGTASMENSTWSWRVPSLIQAVPSILAILVLLALPESPRWLAYQGR
CDEALKVLCAINGREESEPEVQIQYQEIMDNIAFEKSDGQTLGFSEVIKNKSNARRLMLAVSVPPLAML
TGSNIITFYFSTMLEQAGITDASTQLQINVILSAWQLVVALSGSLLAERIGRRMSALSSLGSCTVFFYML
GGLTSKYGNSTNASGIYGTIACIFLFLGAYSFGITPLTVMYQPEVLSYSIRATGMSISTVTSNACGLLVT
FAFPFALDAIGWKTYMINATFNVFLWAFIAYFWVETKGLTLEEIDEIFDGTKHSDMPNLADFHAGRSDV
IGGVEVASPVNVRVPLKL*
SEQ ID NO: 79 G0RGH7-lactose permease
MKEPPKAWTKAQVLVYSFSIIAFFCSTMNGYDGSLINNLLQNPWFKAKYTVGNDGIWAGIVSSMYQIG
GVVALPFVGPAIDGFGRRIGMLLGAILIVVGTIIQGLSNSQGQFMGGRFLLGFGVSIAAAAGPMYVVEI
NHPAYRGRVGAMYNTLWFSGAIISAGAARGGLNVGGDYSWRLITWLQALFSGLIIIFCMFLPESPRWL
YVHHKKDAAKAVLTKYHGNGNPDSVWVQLQLFEYEQLLNMDGADKRWWDYRALFRSRAAVYRLLC
NVTITIFGQWAGNAVLSYFLGSVLDTAGYTGTIAQANITLINNCQQFAWAILGAFLVDRVGRRPLLLFSF
AACTVVWLGMTVASSEFAQSFIGNDANGDPIYSNPSASKAALAMIFIFGAVYSVGITPLQALYPVEVLS
FEMRAKGMAFSSFATNAAGLLNQFAWPVSMDKIGWKTYIIFTIWDLVQTVVVYFFIPETKGRTLEELD
EIFEAKNPVKTSTTKKAVAVDSHGDIVNIEKA*
SEQ ID NO: 80 G0RGH7-lactose permease
MKEPPKAWTKAQVLVYSFSIIAFFCSTMNGYDGSLINNLLQNPWFKAKYTVGNDGIWAGIVSSMYQIG
GVVALPFVGPAIDGFGRRIGMLLGAILIVVGTIIQGLSNSQGQFMGGRFLLGFGVSIAAAAGPMYVVEI
NHPAYRGRVGAMYNTLWFSGAIISAGAARGGLNVGGDYSWRLITWLQALFSGLIIIFCMFLPESPRWL
YVHHKKDAAKAVLTKYHGNGNPDSVWVQLQLFEYEQLLNMDGADKRWWDYRALFRSRAAVYRLLC
NVTITIFGQWAGNAVLSYFLGSVLDTAGYTGTIAQANITLINNCQQFAWAILGAFLVDRVGRRPLLLFSF
AACTVVWLGMTVASSEFAQSFIGNDANGDPIYSNPSASKAALAMIFIFGAVYSVGITPLQALYPVEVLS
FEMRAKGMAFSSFATNAAGLLNQFAWPVSMDKIGWKTYIIFTIWDLVQTVVVYFFIPETKGRTLEELD
EIFEAKNPVKTSTTKKAVAVDSHGDIVNIEKA*
SEQ ID NO: 81 R1GM85-lactose permease
MEKNATTLREESLNGPKEIKIISGTAAFEEAKLKEPPKPFAARSLILYLACLVGFLCSTANGYDGSLMNS
FLETPAFLEFFHIENKGLWSGIVANMYTIGGVVALPFVGPSLDQLGRRAGMFAGATLIIIGTIIQGTTSTD
ASRAQFMGGRFVLGFGVSFMTAGGPILVLEISHPAYRGVMTAWYNTFWFTGSILASGTARGTIGLHG
NNSWLIMTWLQLLFAGVVFLFAWILPESPRWLYTRGKRDKCREMLTYWHGHDNPDSVWVQLQLQE
YEEYLEMDGSDKRWWDYRSLFRNKPSVYRLCCNLVIVVFGQWAGNAVLSYYLSSALDTAGYHDELQ
QKNINLILNCVQFVTALIGARTVEWFGRRPLLLFANIGCAICWVCITGSTATLAKDETNNAAGTAAVAFI
FMFNIIFAFGFTPLQQLVPVEVLSFEMRAKGMAFSSFVMNLAMLMNNYAWPVSMEKIGWRTYIIFAVW
DVFQAIVIYFLIPETKNRTLEELDDIFHASNPVKASLEKKRIAVDSDRHVLEVEKS*
SEQ ID NO: 82 R1GM85-lactose permease
MEKNATTLREESLNGPKEIKIISGTAAFEEAKLKEPPKPFAARSLILYLACLVGFLCSTANGYDGSLMNS
FLETPAFLEFFHIENKGLWSGIVANMYTIGGVVALPFVGPSLDQLGRRAGMFAGATLIIIGTIIQGTTSTD
ASRAQFMGGRFVLGFGVSFMTAGGPILVLEISHPAYRGVMTAWYNTFWFTGSILASGTARGTIGLHG
NNSWLIMTWLQLLFAGVVFLFAWILPESPRWLYTRGKRDKCREMLTYWHGHDNPDSVWVQLQLQE
YEEYLEMDGSDKRWWDYRSLFRNKPSVYRLCCNLVIVVFGQWAGNAVLSYYLSSALDTAGYHDELQ
QKNINLILNCVQFVTALIGARTVEWFGRRPLLLFANIGCAICWVCITGSTATLAKDETNNAAGTAAVAFI
FMFNIIFAFGFTPLQQLVPVEVLSFEMRAKGMAFSSFVMNLAMLMNNYAWPVSMEKIGWRTYIIFAVW
DVFQAIVIYFLIPETKNRTLEELDDIFHASNPVKASLEKKRIAVDSDRHVLEVEKS*
SEQ ID NO: 83 M5G759-lactose permease
MAKHKPNPLSTSMLLLYPILLVAFMNSAANGFDGNTFGGVSAIADFQARFGTNVAASDGFLAAIYILGN
VIGSFVAGPAADWLGRKRGMILANIIVLIGTIVQAAAMQRRDMIAGRVVLGIGSVMLGPSATSYVVEMS
YPAYRGTIVGLYNGCYFIGAIVSTWLEYGLVDDTKGEINWRIPMAMQGIPCIIVLAFVWFLPESPRWLM
ARGREEEAKRILIKYHGEGDPDNELVQLEMEEMHEAIDTAGSDTRWWDYRELFNTRGARHRMFLVL
CVGFFGQIDLPPTSYYMPLMAQTAGIVSTKQQLLMNALQSPVMTIGTLLGVHTIDKYGRRPMLIVSSA
VCSLCVLIIIICSLKQEGHPSVGLAGISFVYVFLFAFAFVWTPMQALYPSEVLAYNARAKGLGMSGLWI
NIVSFINTYAAPVGITNSGWKFYFLYFVIDVVGIITIYFFFIETKDRSLEEIDEIFADPHPVRTSLRKQKISV
AIEKS*
SEQ ID NO: 84 M5G759-lactose permease
MAKHKPNPLSTSMLLLYPILLVAFMNSAANGFDGNTFGGVSAIADFQARFGTNVAASDGFLAAIYILGN
VIGSFVAGPAADWLGRKRGMILANIIVLIGTIVQAAAMQRRDMIAGRVVLGIGSVMLGPSATSYVVEMS
YPAYRGTIVGLYNGCYFIGAIVSTWLEYGLVDDTKGEINWRIPMAMQGIPCIIVLAFVWFLPESPRWLM
ARGREEEAKRILIKYHGEGDPDNELVQLEMEEMHEAIDTAGSDTRWWDYRELFNTRGARHRMFLVL
CVGFFGQIDLPPTSYYMPLMAQTAGIVSTKQQLLMNALQSPVMTIGTLLGVHTIDKYGRRPMLIVSSA
VCSLCVLIIIICSLKQEGHPSVGLAGISFVYVFLFAFAFVWTPMQALYPSEVLAYNARAKGLGMSGLWI
NIVSFINTYAAPVGITNSGWKFYFLYFVIDVVGIITIYFFFIETKDRSLEEIDEIFADPHPVRTSLRKQKISV
AIEKS*
SEQ ID NO: 85 A0A16216F8-lactose permease
MAIDEQKAHIAHDESIADEKMTGNVKTIGTGSVALAAAVASQKPRLLSKNMIQLYLIMGVGYLVSTMNG
FDSSLMGSINAMKPYQESFGLAGAGSTTGIIFIIYNLGQIAAFPFCGLLADGYGRRLCIFVGCLVVVAGT
AVQGSAHSLGQFVGGRFLLGFGAAIASAAGPAYTVELAHPAYRGFMAGMYNNFWWLGNILAGWTS
YASNKHLQSSWAWRVPTIVQAGLPGVVMVLILFFPESPRWLIANDRAEEALAILAKYHGDGDANSAIP
TLEYNEIVEMNRLGKDDNPWWDFRELWNTRAARYRLGMVVGMAFIGQWSGNNVVTYFMPEMIVQA
GITDTNKQLLLNAINPIFCMLGAVYGASLLDRLGRRTMMLAGLVGALASYCMLTAFTAEAERHASLAY
GVIASIYIFGIFFSWGFTPLQTLYAVECLENRTRAKGSGLNFLFLNIAMVVNTYGISVGMQQLGWKLYL
VYIGWICVEMLIIYFFFVETAGKTLEQLKDVFEAKNPVKASIAKTNVELDESGRIVGVVGEGV*
SEQ ID NO: 86 A0A194WU59-lactose permease
MASDEKDLGAASHVNDIDNSSTDAILLATEADTTTYSPWSRSMFRLYGVLAIAYLCGCLNGFDGSLM
GAINAMKPYQNYFGIGSTGSSTGIVFAIYNIGSIPAVVLTGPVNDYLGRRAGMFTGSVIIIIGTCIQAPAV
NMHMFLAGRFLLGFGVSFCCVSAPCYVSELAHPKWRGTLTGLYNTCWYIGSIIASWTCYGTAFLSTN
WSWRIPIWCQLLSSVVVAGGAFFLPESPRWLVGQDRVDEARAILAKYHGEGREDHPIVNLQISEMYY
QIQTDATDKRWWDYSGLVKSHNARRRLICVLGMAFFGQWSGNSVSSYYFPEMMATAGIADEHTQLK
LNGVFPVLCWLGAISGARMTDKIGRRPLLLWSILFSSICFAIITGTTKLAVEHNNKMASNVAITFVYLFGI
VFSFGWTPLQSMYIAETLPTETRAKGTAIGNFGSSVASTVIQYSSGPAFKNITYYFYLVFVAWDLIEFV
VIYFFFVETKDRTLEELDEVFSDLHPVKKSLQKRSVQTVAATVGVDVNEKSMVA*
SEQ ID NO: 87 A0A194WU59-lactose permease
MASDEKDLGAASHVNDIDNSSTDAILLATEADTTTYSPWSRSMFRLYGVLAIAYLCGCLNGFDGSLM
GAINAMKPYQNYFGIGSTGSSTGIVFAIYNIGSIPAVVLTGPVNDYLGRRAGMFTGSVIIIIGTCIQAPAV
NMHMFLAGRFLLGFGVSFCCVSAPCYVSELAHPKWRGTLTGLYNTCWYIGSIIASWTCYGTAFLSTN
WSWRIPIWCQLLSSVVVAGGAFFLPESPRWLVGQDRVDEARAILAKYHGEGREDHPIVNLQISEMYY
QIQTDATDKRWWDYSGLVKSHNARRRLICVLGMAFFGQWSGNSVSSYYFPEMMATAGIADEHTQLK
LNGVFPVLCWLGAISGARMTDKIGRRPLLLWSILFSSICFAIITGTTKLAVEHNNKMASNVAITFVYLFGI
VFSFGWTPLQSMYIAETLPTETRAKGTAIGNFGSSVASTVIQYSSGPAFKNITYYFYLVFVAWDLIEFV
VIYFFFVETKDRTLEELDEVFSDLHPVKKSLQKRSVQTVAATVGVDVNEKSMVA*
SEQ ID NO: 88 A0A1S8BLP4-lactose permease
MAFLPNFEATPLAPPHSSRLVIALLILSSCISSATLGYDGSMMSGLNILPAYNDYFHLTPATTALNTATV
YIGQVIPCFFYGAVSDRLGRKNAMAIAAVATIAAVVLQAAAQNVAMFAVSRILVGVGNGATSIAGPVWL
SECLPHRWRAWGLGFRIGYVGGLIASGITYGTGMMNSTWAWRLPSAVQGIFSVLCILILPFVPESPR
WLVYQNRGEEALYALALTHSHGDQKDAATLVEFQ*
SEQ ID NO: 89 E9EYU8-lactose permease
MAEKVLPVASAHINTDAKQVETRVVTGDEAFNEALLKEPPTPWSRGQLMIYLFSLVAFFNSTVNGYD
GSLINNLLQNPSFIAKYNGSNSGIWAGIVSSMYQIGNIVALPFLGPVCDHFGRRAGMASGSAIIIIGTIIQ
GTSNAAGQFMGGRFLLGFGVSLVATGGPMYVVEINHPAYRGVVGAMYNTLWFSGSILSSGAARGAA
NVGGDYSWRLITWLQQILFPSLVLIFSFLLPESPRWLYVHNRKEKAKAILVKYHGNGNDSSPWVSLQL
REYEECLNMNGADKRWWDYRVLFRRGNFYRLSCNLIVSAFGQLAGNAVLSYFLGSVLDSAGYTSYL
AQANITLINNCVQFLCAICGALLVDRIGRRPLLLFAFTSCTVVWLGMTIAASQFAASYAGETASGVYVY
TNGAASKAALAMIFLFGAAFSIGITPLQGLYIVEVLSFEMRAKGMAMSNLAVNLAGLLNQYAWSVSMK
NIGWKTYIIFTVWDAISVIIIYFTLPETKGRTLEELDQIFAAKNPVKASLSKKEILVSRDGDVVDVKEASP*
SEQ ID NO: 90 E9EYU8-lactose permease
MAEKVLPVASAHINTDAKQVETRVVTGDEAFNEALLKEPPTPWSRGQLMIYLFSLVAFFNSTVNGYD
GSLINNLLQNPSFIAKYNGSNSGIWAGIVSSMYQIGNIVALPFLGPVCDHFGRRAGMASGSAIIIIGTIIQ
GTSNAAGQFMGGRFLLGFGVSLVATGGPMYVVEINHPAYRGVVGAMYNTLWFSGSILSSGAARGAA
NVGGDYSWRLITWLQQILFPSLVLIFSFLLPESPRWLYVHNRKEKAKAILVKYHGNGNDSSPWVSLQL
REYEECLNMNGADKRWWDYRVLFRRGNFYRLSCNLIVSAFGQLAGNAVLSYFLGSVLDSAGYTSYL
AQANITLINNCVQFLCAICGALLVDRIGRRPLLLFAFTSCTVVWLGMTIAASQFAASYAGETASGVYVY
TNGAASKAALAMIFLFGAAFSIGITPLQGLYIVEVLSFEMRAKGMAMSNLAVNLAGLLNQYAWSVSMK
NIGWKTYIIFTVWDAISVIIIYFTLPETKGRTLEELDQIFAAKNPVKASLSKKEILVSRDGDVVDVKEASP*
SEQ ID NO: 91 A0A136IU29-lactose permease
MSSPTGETKPELHGEEKEFGEVRHVNAASIALAAAVAEQKPNPWSRNMISLYMIMAIGYLVSTMNGF
DSSLMGAINAMDEYTTMFNLQGDDGNSTGIIFIIYNIGQIASFPFCGLLADGLGRRWCIFIGCAIVLVGT
AIQTTAHERGQFIGGRFVLGFGASIASAAGPAYIVELAHPAYRGTMAGMYNNFWWLGNIIAGWTTYG
TEYHLKNSWAWRAPTVVQCIMPAIVMSLIMFFPESPRWLIHHDRTEEALAIFAKYHGDGDEQSALVQL
QYREIVAERAATQNPNAWWDLRELCNTKAARYRMFMVIGMSFFGQWSGNNVVSYFMPVMVEQAGI
KDKSTQLLINAINPIFSMIAAIYGATLLDKLGRRFMMLAGLGGALVFYCMLTGFTAGSETNKNLSYGVIV
SIYLFGVCFAWGFTPLQTLYAVECLENRTRAKGSGANFLFLNIAMVVNTYGISVGMKAIGWKLYIVYIV
WIMIEMVIIYFFFVETAGKTLEEMSEIFEAPNPRKASTKKTKMGLNQAGQVVGVDEESH*
SEQ ID NO: 92 A0A136IU29-lactose permease
MSSPTGETKPELHGEEKEFGEVRHVNAASIALAAAVAEQKPNPWSRNMISLYMIMAIGYLVSTMNGF
DSSLMGAINAMDEYTTMFNLQGDDGNSTGIIFIIYNIGQIASFPFCGLLADGLGRRWCIFIGCAIVLVGT
AIQTTAHERGQFIGGRFVLGFGASIASAAGPAYIVELAHPAYRGTMAGMYNNFWWLGNIIAGWTTYG
TEYHLKNSWAWRAPTVVQCIMPAIVMSLIMFFPESPRWLIHHDRTEEALAIFAKYHGDGDEQSALVQL
QYREIVAERAATQNPNAWWDLRELCNTKAARYRMFMVIGMSFFGQWSGNNVVSYFMPVMVEQAGI
KDKSTQLLINAINPIFSMIAAIYGATLLDKLGRRFMMLAGLGGALVFYCMLTGFTAGSETNKNLSYGVIV
SIYLFGVCFAWGFTPLQTLYAVECLENRTRAKGSGANFLFLNIAMVVNTYGISVGMKAIGWKLYIVYIV
WIMIEMVIIYFFFVETAGKTLEEMSEIFEAPNPRKASTKKTKMGLNQAGQVVGVDEESH*
SEQ ID NO: 93 A0A136IRI6-lactose permease
MGLFKRNKTEAETAVGPALAAASSSVVLRLPLVLPNDPRPFYKVRHLLLLNLLLLLTSLSSASIGFDGA
MMNGLQTVAQWRDYFGRPTPAILGVMNAIYPIGKLVGIFPTTWLADKYGRKAPMYLGFVMLVVGAGI
QGGSVHIGMFIASRFFLGFGTAFLAQPAPILVTELAYPTHRGKITAIYQTFFFFGAILAAWSTYGTLRIPS
TWSWRIPSLLQGAIPAFQLALFWFVPESPRWLVAKGREAEARVILTKWHSAGDESSPLVDYEMTQIK
ETVQLETEALSETSYLDLVRTPANRRRTFIAVIVGFFAQWNGAGVISYYLALVLNTIGITESRDQALING
LLQVFNWLAAIFAGALMVDRLGRRTLFLASTAGMFFSYVIWTALTGVFTTTLNQNMGNAVVAFIFIYYF
FYDIAWNPLLLAYPVEVFQFTLRARGVSVTYASTFIGLIIGQFVNPLAMAGLGWKYYIVFCVILACLFVVI
WFTFPETKGRTLEEIAEIFDGPGAGHGAAATDEEAAAAEAKASSIENEHEDVSVPRKA*
SEQ ID NO: 94 A3GIC4-lactose permease
MSTNSLNDSYNPSSTKEKDIVVQSEALADVAIETAFETDGYKKIFQEHPVPRWTKSRLSIYFTCLVIYLV
STTNGYDGSLLSSLITMPEFISHLNIKSASGTGIVFAIFQVGQMVATLFVWLGDFIGRRNAIFIGSVIVCL
GAIITSIANNTSTFIGGRFLLSFGSGISCALSTTYLLEITSPDERSALCAIYNSLYYIGSIIATWSSYATSISY
ANSVLSFRIPLWLQILCPALVVIGLLVGVAPESPRFYYLTGQPDKARAFFCKYHANGDEKHPIVEYEMA
QLELSLLEVPKLRVRDYFDARILFKTKSRIYRSLVCIAHSAFGQLSGNAVVGYYITNIFLELGITNPTTRL
LLNGVNSILGFIFAMSGSILVGRIGRRPILLYSTTGFVISFTIIAACIAAYTNNNNQVAAKVGIAFIYIFNNVF
FSFGYTPLQPLYPAEILSSEMRAKGMALFQITQGTASFINTYAAPVAMQNIKYWYYVFFVFWDTFEVIII
YLYFVETKNLTLEEIELIFESATPVKTSMIISKPGHAANEEKLRLANLKLGKNYVA*
SEQ ID NO: 95 A3GIC4-lactose permease
MSTNSLNDSYNPSSTKEKDIVVQSEALADVAIETAFETDGYKKIFQEHPVPRWTKSRLSIYFTCLVIYLV
STTNGYDGSLLSSLITMPEFISHLNIKSASGTGIVFAIFQVGQMVATLFVWLGDFIGRRNAIFIGSVIVCL
GAIITSIANNTSTFIGGRFLLSFGSGISCALSTTYLLEITSPDERSALCAIYNSLYYIGSIIATWSSYATSISY
ANSVLSFRIPLWLQILCPALVVIGLLVGVAPESPRFYYLTGQPDKARAFFCKYHANGDEKHPIVEYEMA
QLELSLLEVPKLRVRDYFDARILFKTKSRIYRSLVCIAHSAFGQLSGNAVVGYYITNIFLELGITNPTTRL
LLNGVNSILGFIFAMSGSILVGRIGRRPILLYSTTGFVISFTIIAACIAAYTNNNNQVAAKVGIAFIYIFNNVF
FSFGYTPLQPLYPAEILSSEMRAKGMALFQITQGTASFINTYAAPVAMQNIKYWYYVFFVFWDTFEVIII
YLYFVETKNLTLEEIELIFESATPVKTSMIISKPGHAANEEKLRLANLKLGKNYVA*
SEQ ID NO: 96 A3LPQ5-lactose permease
MSSEMLSKSEVKYEQNEMEGSQEKLALKDEDSKDFYKVNEAYNEKGFPLLSRPMIPLLLTCSVVYFV
STNTGFDGSLMSSIYTQQDYLDKFNLSINSSTSTGLVFSIYNVAQICAAFFCPLIDFWGRKKLILIGCWG
TVLGAIITAFAQNKETLIAGRFVLSFFTTLANTSASLYVTEIANTYNRSVVAGCYNTLWYIGSVLAAFTSY
GANVNLGGTELAFRLPLGIQAVFPGLVGIFGFFIPESPRWLVGVGREKEAEEMIAKYHONGDFSHPLL
EHEMVQINESFRGNKLAQSLKILDLRPIFQNNNAYRSILVILMAFFGQFSGNNVCSYYLPTMLRNIGMT
TVSTNVLMNAFYSLI*
SEQ ID NO: 97 A3LPQ5-lactose permease
MSSEMLSKSEVKYEQNEMEGSQEKLALKDEDSKDFYKVNEAYNEKGFPLLSRPMIPLLLTCSVVYFV
STNTGFDGSLMSSIYTQQDYLDKFNLSINSSTSTGLVFSIYNVAQICAAFFCPLIDFWGRKKLILIGCWG
TVLGAIITAFAQNKETLIAGRFVLSFFTTLANTSASLYVTEIANTYNRSVVAGCYNTLWYIGSVLAAFTSY
GANVNLGGTELAFRLPLGIQAVFPGLVGIFGFFIPESPRWLVGVGREKEAEEMIAKYHCNGDFSHPLL
EHEMVQINESFRGNKLAQSLKILDLRPIFQNNNAYRSILVILMAFFGQFSGNNVCSYYLPTMLRNIGMT
TVSTNVLMNAFYSLI*
SEQ ID NO: 98 G8XV57-lactose permease
MADEKTVQPSAEQEAVGEAEEVRVAHDALNAKYSPLTWSMFRLYLCLIIPYLCGTLNGYDGSLMGGL
NAMETYLDFFNMETSGSSTGIVFALYNIGSIPAVFFTGPVNDYWGRRCGMFVGALIIVIGTCIQSPSVN
RGMFLAGRFILGFGVSFCCVSAPCYVSEMAHPAWRGTITGLYNCTWYIGSILASWVVYGCSQLDNAN
SFRIPIWCQLISSALVVLGVWFIPESPRWLMAQDRAEDAAKILTRYHGENDPDHPLVHLQLKEMQQSI
ATDASDKKWWDYRELYTGHSARRRLICVLGMACFGQISGNSVTSYYLPVMLENAGIVSESRKLLENG
IYPPLSLIGAVVGARMTDTIGRRPLLIYSLLFCSVAFAIITGTSKLATDDPTNTAAANTTIAFIYLFGIVFSF
GWTPLQSMYIAETLTTTTRAKGTAVGNLASSIASTIIQYSSGPAFKDIQYYFYLVFVFWDLIEIVIMYFYF
PETKDRTLEELEEVFSAPNPVKRSLVKRDAATVLNTMQVEQRELVSKEAQV*
SEQ ID NO: 99 Q5B9G6-lactose permease
MGEINEEKHDISVTEGAKVATMHGMTAEKPGATTKSVFNAELFAAINETKIERWSKTSIHLYCAVCIFV
SFCCACANGYDGSLMGAVFAMDHYQATFNTGMTGQKVSVVTSLYTVGSMVATPFSAVISDNFGRRK
CMFVGGWVIIIGSIVIATASTLAHFIVGRFILGFGIQIMVVSAPAYAAEISPPHWRGRAVGLYNCGWFGG
SIPAACVTYGCNYIDSNWSWRVPFLLQCFASVIVIISVWFIPESPRWLIAHGKEEEAIAILAKYHGNGDP
NARLVRLEADEMREGIRQDGIDKRWWDYRPFLLSHNGRWRFAQVIMISIFGQWSGNGLGYFNPAIYE
ALGYTSSSMQLLLNLVNSIVGAIGALTAVYYCDRMPRRTVLVWGTLGCAICMAVNAGVSQPLIPQRNA
GETLDPTFGRTALAFYYLFQVVFSFTYTPLQGVVPAEALETTTRAKGLALSGFLVSGTSFISQYASPIA
LGNISTNYFWIFVGWDVVETACWYLFGVEAQGRTLEELEYIYNQPYPVKASKKRDRVVVQQDGHVTE
KISADEA*
SEQ ID NO: 100 Q9MLU0-Fucose synthase
MADNTGSEMKSGSFMLEKSAKIFVAGHRGLVGSAIVRKLQDQGFTNLVLRTHSELDLTSQ
SDVESFFATEKPVYVILAAAKVGGIHANNTYPADFIGVNLQIQTNVIHSAYTHGVKKLLF
LGSSCIYPKFAPQPIPESALLTGPLEPTNEWYAIAKIAGIKMCQAYRLQHQWDAISGMPT
NLYGQNDNFHPENSHVLPALMRRFHEAKANNADEVVVWGSGSPLREFLHVDDLADACVFL
MDQYSGFEHVNVGSGVEVTIKELAELVKEVVGFKGKLVWDTTKPDGTPRKLMDSSKLASL
GWTPKISLKDGLSQTYEWYLENVVQKKQ*
SEQ ID NO: 101 O49213- Fucose synthase
MAETIGSEVSSMSDKSAKIFVAGHRGLVGSAIVRKLQEQGFTNLVLKTHAELDLTRQADV
ESFFSQEKPVYVILAAAKVGGIHANNTYPADFIGVNLQIQTNVIHSAYEHGVKKLLFLGS
SCIYPKFAPQPIPESALLTASLEPTNEWYAIAKIAGIKTCQAYRIQHGWDAISGMPTNLY
GPNDNFHPENSHVLPALMRRFHEAKVNGAEEVVVWGTGSPLREFLHVDDLADACVFLLDR
YSGLEHVNIGSGQEVTIRELAELVKEVVGFEGKLGWDCTKPDGTPRKLMDSSKLASLGWT
PKVSLRDGLSQTYDWYLKNVCNR*
SEQ ID NO: 102 Q13630-Fucose synthase
MGEPQGSMRILVTGGSGLVGKAIQKVVADGAGLPGEDWVFVSSKDADLTDTAQTRALFEK
VQPTHVIHLAAMVGGLFRNIKYNLDFWRKNVHMNDNVLHSAFEVGARKVVSCLSTCIFPD
KTTYPIDETMIHNGPPHNSNFGYSYAKRMIDVQNRAYFQQYGCTFTAVIPTNVFGPHDNF
NIEDGHVLPGLIHKVHLAKSSGSALTVWGTGNPRRQFIYSLDLAQLFIWVLREYNEVEPI
ILSVGEEDEVSIKEAAEAVVEAMDFHGEVTFDTTKSDGQFKKTASNSKLRTYLPDFRFTP
FKQAVKETCAWFTDNYEQARK*
SEQ ID NO: 103 P32055-Fucose synthase
MSKQRVFIAGHRGMVGSAIRRQLEQRGDVELVLRTRDELNLLDSRAVHDFFASERIDQVY
LAAAKVGGIVANNTYPADFIYQNMMIESNIIHAAHQNDVNKLLFLGSSCIYPKLAKQPMA
ESELLQGTLEPTNEPYAIAKIAGIKLCESYNRQYGRDYRSVMPTNLYGPHDNFHPSNSHV
IPALLRRFHEATAQNAPDVVVWGSGTPMREFLHVDDMAAASIHVMELAHEVWLENTQPML
SHINVGTGVDCTIRELAQTIAKVVGYKGRVVFDASKPDGTPRKLLDVTRLHQLGWYHEIS
LEAGLASTYQWFLENQDRFRG*
SEQ ID NO: 104 Wildtype Neisseria meningitidis LgtA β-1,3-N-
acetylglucosaminyltransferase
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 105 Nme.LgtA_A258D
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFWYDVSKLGRLAYYPEALVKYRLHANQVSSKYSIRQHEIAQGIQKTDRNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 106 Nme.LgtA_c.945delA
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPMPVPG*
SEQ ID NO: 107 Nme.LgtA_E294N.c890addT
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPNEDFRKS*
SEQ ID NO: 108 Nme.LgtA_G179R
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFRNPIHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 109 Nme.LgtA_K242H
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFWYDVSKLGRLAYYPEALVKYRLHANQVSSHYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 110 Nme.LgtA_L229P
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFWYDVSKLGRLAYYPEAPVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 111 Nme.LgtA_M187P
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTPIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 112 Nme.LgtA_N185G
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNGTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 113 Nme.LgtA_P89T and G179R
MPSEAFRRHRAYRENKLQSLVSVLICAYNVEKYFAQSLAAVVNQTWRNLEILIVDDGSTDGTLAIAKD
FQKRDSRIKILAQAQNSGLITSLNIGLDELAKSGMGEYIARTDADDIAAPDWIEKIVGEMEKDRSIIAMG
AWLEVLSEEKDGNRLARHHRHGKIWKKPTRHEDIADFFPFRNPIHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 114 Nme.LgtA_Q211V
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYVFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 115 Nme.LgtA_S240V
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFWYDVSKLGRLAYYPEALVKYRLHANQVVSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 116 Nme.LgtA_W213N
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFGNPIHNNTMIMRRSVIDGGLRYNTERD
WAEDYQFNYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR*
SEQ ID NO: 117 Nme.LgtA_G179R_E170L
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHLDIADFFPFRNPIHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR
SEQ ID NO: 118 Nme.LgtA_G179R_I182V
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFRNPVHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR
SEQ ID NO: 119 Nme.LgtA_G179R_V216L
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFRNPIHNNTMIMRRSVIDGGLRYNTERD
WAEDYQFWYDLSKLGRLAYYPEALVKYRLHANQVSSKYSIRQHEIAQGIQKTARNDFLQSMGFKTRF
DSLEYRQIKAVAYELLEKHLPEEDFERARRFLYQCFKRTDTPPAGAWLDFAADGRMRRLFTLRQYFG
ILRRLLKNR
SEQ ID NO: 120 Nme.LgtA_G179R_K290Q
MPSEAFRRHRAYRENKLQSLVSVLICAYNVEKYFAQSLAAVVNQTWRNLEILIVDDGSTDGTLAIAKD
FQKRDSRIKILAQAQNSGLIPSLNIGLDELAKSGMGEYIARTDADDIAAPDWIEKIVGEMEKDRSIIAMG
AWLEVLSEEKDGNRLARHHRHGKIWKKPTRHEDIADFFPFRNPIHNNTMIMRRSVIDGGLRYNTERD
WAEDYQFWYDVSKLGRLAYYPEALVKYRLHANQVSSKYSIRQHEIAQGIQKTARNDFLQSMGFKTRF
DSLEYRQIKAVAYELLEQHLPEEDFERARRFLYQCFKRTDTPPAGAWLDFAADGRMRRLFTLRQYFG
ILRRLLKNR
SEQ ID NO: 121 LgtB from Pasteurella multocida
MSGEHYVISLSSAVERRQHIRNQFSQKNIPFQFFDAISPSPLLDQLVLQFFPRLADSSLTGGEKACFM
SHLSLWHKCVEENLPYIVVFEDDIVLGKDADKFLIGDEWLFSRFDPEEIFIIRLETFLQKVVCESTHIAPY
THRDFLSLKSAHFGTAGYVISQGAAKFLLDIFKNISNEHIAPIDELIFNQFLVKNSFNVYQLSPAICVQEL
QLNNESSALQSQLELERNKFRNKKSEELKRNRKNFIEKFIYILKKPKRMLDNNKRKREESKIENDKMIIE
FK
SEQ ID NO: 122 LgtB from Neisseria gonorrhoeae
MQNHVISLASAAERRAHIAATFGSRGIPFQFFDALMPSERLERAMAELVPGLSAHPYLSGVEKACFMS
HAVLWEQALDEGVPYIAVFEDDVLLGEGAEQFLAEDTWLQERFDPDSAFVVRLETMFMHVLTSPSG
VADYGGRAFPLLESEHCGTAGYIISRKAMRFFLDRFAVLPPERLHPVDLMMFGNPDDREGMPVCQL
NPALCAQELHYAKFHDQNSALGSLIEHDRRLNRKQQWRDSPANTFKHRLIRALTKIGREREKRRQRR
EQLIGKIIVPFQ

1. A host cell capable of producing a human milk oligosaccharide (HMO), wherein the host cell comprises one or more heterologous nucleic acids that each, independently, encode:
(a) a fucosyltransferase having an amino acid sequence that is at least 90% identical to the amino acid sequence of any one of SEQ ID NOS: 1-41; and/or
(b) a GDP-mannose dehydratase (GMD) having an amino acid sequence that is at least 90% identical to the amino acid sequence of any one of SEQ ID NOS: 42-64; and/or
(c) a lactose permease having an amino acid sequence that is at least 90% identical to the amino acid sequence of any one of SEQ ID NOS: 65-99; and/or
(d) a fucose synthase having an amino acid sequence that is at least 90% identical to the amino acid sequence of any one of SEQ ID NOS: 100-103.

2. A host cell capable of producing a HMO, wherein the host cell comprises one or more heterologous nucleic acids that each, independently, encode a fucosyltransferase, a GMD, a lactose permease, and/or a fucose synthase, wherein the host cell produces the HMO;
(a) at a yield of at least 20% (w/w): or
(b) at a productivity of at least 1 g/L/hr.

3-15. (canceled)

16. The host cell of claim 1, wherein the fucosyltransferase has the amino acid sequence of any one of SEQ ID NOS: 1-3.

17-21. (canceled)

22. The host cell of claim 1, wherein the GMD has the amino acid sequence of any one of SEQ ID NOS: 42-44.

23-24. (canceled)

25. The host cell of claim 1, wherein the lactose permease has the amino acid sequence of any one of SEQ ID NOS: 65-99.

26-27. (canceled)

28. The host cell of claim 1, wherein the fucose synthase has the amino acid sequence of any one of SEQ ID NOS: 100-103.

29-30. (canceled)

31. The host cell of claim 1, wherein the HMO is a reducing sugar or comprises a fucose residue.

32. (canceled)

33. The host cell of claim 1, wherein the HMO is lacto-N-neotetraose (LNnT), 2′-fucosyllactose (2′-FL), 3-fucosyllactose (3-FL), difucosyllactose (DFL), lacto-N-tetraose (LNT), lacto-N-fucopentaose (LNFP) I, LNFP II, LNFP III, LNFP V, LNFP VI, lacto-N-difucohexaose (LNDFH) I, LNDFH II, lacto-N-hexaose (LNH), lacto-N-neohexaose (LNnH), fucosyllacto-N-hexaose (F-LNH) I, F-LNH II, difucosyllacto-N-hexaose (DFLNH) I, DFLNH II, difucosyllacto-N-neohexaose (DFLNnH), difucosyl-para-lacto-N-hexaose (DF-para-LNH), difucosyl-para-lacto-N-neohexaose (DF-para-LNnH), trifucosyllacto-N-hexaose (TF-LNH), 3′-siallylactose (3′-SL), 6′-siallylactose (6′-SL), sialyllacto-N-tetraose (LST) a, LST b, LST c, disialyllacto-N-tetraose (DS-LNT), fucosyl-sialyllacto-N-tetraose (F-LST) a, F-LST b, fucosyl-sialyllacto-N-hexaose (FS-LNH), fucosyl-sialyllacto-N-neohexaose (FS-LNnH) I, or fucosyl-disialyllacto-N-hexaose (FDS-LNH) II.

34. The host cell of claim 1, wherein the host cell further comprises:
(a) one or more of a β-1,3-N-acetylglucosaminyltransferase (LgtA), a β-1,4-galactosyltransferase (LgtB), and a UDP-N-acetylglucosamine diphosphorylase;
(b) a fucosidase; or
(c) a protein that transports lactose into the host cell, optionally wherein the protein that transports lactose into the cell is an active transporter.

35-43. (canceled)

44. The host cell of claim 34, wherein;
(a) the LgtA has an amino acid sequence that is at least 85% identical to the amino acid sequence of any one of SEQ ID NOS: 105-120; or
(b) the LgtB has an amino acid sequence that is at least 85% identical to the amino acid sequence of SEQ ID NO: 121 or SEQ ID NO: 122.

45-58. (canceled)

59. The host cell of claim 1, wherein the host cell further comprises a heterologous nucleic acid encoding one or more of a prostate specific antigen-1 (PSA1), phosphomannomutase SEC53 (SEC53), uroporphyrinogen decarboxylase Hem12 (HEM12), SNF1-activating kinase 1 (SAK1), acetyl-coenzyme A synthetase 1 (ACS1), cell wall protein DAN1 (DAN1), or pro-neuropeptide Y (NYP1) proteins.

60-64. (canceled)

65. The host cell of claim 1, wherein the host cell produces the HMO at a yield of at least 20% (w/w), optionally wherein the host cell produces the HMO at a yield of between 20% (w/w) and 70% (w/w).

66-67. (canceled)

68. The host cell of claim 1, wherein the host cell produces the HMO at a productivity of between at least 1 g/L/hr, optionally wherein the host cell produces the HMO at a productivity of between 1 g/L/hr and 5 g/L/hr.

69-70. (canceled)

71. The host cell of claim 1, wherein the host cell is a yeast cell.

72-74. (canceled)

75. A method of producing a HMO, the method comprising culturing a population of host cells of claim 1 in a culture medium under conditions suitable for the host cells to produce the HMO.

76. A method of genetically modifying a host cell to be capable of producing a HMO, the method comprising introducing into the host cell one or more heterologous nucleic acids that each, independently, encode:
(a) a fucosyltransferase having an amino acid sequence that is at least 90% identical to the amino acid sequence of any one of SEQ ID NOS: 1-41; and/or
(b) a GMD having an amino acid sequence that is at least 90% identical to the amino acid sequence of any one of SEQ ID NOS: 42-64; and/or
(c) a lactose permease having an amino acid sequence that is at least 90% identical to the amino acid sequence of any one of SEQ ID NOS: 65-99; and/or
(d) a fucose synthase having an amino acid sequence that is at least 90% identical to the amino acid sequence of any one of SEQ ID NOS: 100-103.

77-142. (canceled)

143. A fermentation composition comprising (i) a population of host cells of claim 1 and (ii) a culture medium comprising a HMO produced from the host cells.

144. (canceled)

145. The fermentation composition of claim 143, wherein the HMO is LNnT, 2′-FL, 3-FL, DFL, LNT, LNFP I, LNFP II, LNFP III, LNFP V, LNFP VI, LNDFH I, LNDFH II, LNH, LNnH, F-LNH I, F-LNH II, DFLNH I, DFLNH II, DFLNnH, DF-para-LNH, DF-para-LNnH, TF-LNH, 3′-SL, 6′-SL, LST a, LST b, LST c, DS-LNT, F-LST a, F-LST b, FS-LNH, FS-LNnH I, or FDS-LNH II.

146. The fermentation composition of claim 145, wherein the HMO is 2′FL or 6′SL.

147. (canceled)

148. A composition comprising a mixture comprising at least 85% (v/v) of 2′FL and less than 15% (v/v) of DFL, optionally wherein the mixture comprises from 85% (v/v) to 99% (v/v) of 2′FL and from 15% (v/v) to 1% (v/v) of DFL.


SEQUENCE LISTING (122 sequences)

SEQ ID NO: 1 - protein, 292 aa, unidentified
  source 1..292: note = C. moranbacterium
MIIVKLMGGMGNQLFQYAIGRSLAIRNESEFKMDILGYADQGELLTPRLYALNIFSVQENFASEKEIEKLKSNTSGSVVLALQRFGFFKKSNSFVIEPHFNFSSEILESGNNIYLQGYWQTEKYFKDVEDVIRKEFTLKEKFSIEEKEITKEIKNSNAVSLHIRRGDYVSSATTSKFHGICSLDYYEKAVRHIAEKTENPVFYIFSDDIAWVKENLKIDFPTKYVSDGILKDYEELTLMSYCKHNIIANSSFSWWGAWLNANPEKIVIAPKQWFADQSVNTSDVVPETWVKM

SEQ ID NO: 2 - protein, 293 aa, Herbaspirillum sp.
  source 1..293: note = YR52
MIATRLIGGLGNQMFQYAAGRALALRVGSPLLLDVSGFANYELRRYELDGFRIDATAASAQQLARLGVNATPGTSLLARVLRKVWPQPADRILREASFTYDARIEQASAPVYLDGYWQSERYFARIRQHLLDEFTLKGDWGSDNAAMAAQIATAGAGAVSLHVRRGDYVSNAHTAQYHGVCSLDYYRDAVAHIGGRVEAPHFFVFSDDHEWVRENLQIGHPATFVQINSADHGIYDMMLMKSCRHHIIANSSFSWWGAWLNPAEDKIVVAPQRWFKDATNDTRDLIPAAWVRL

SEQ ID NO: 3 - protein, 291 aa, Sulfurovum lithotrophicum
MIIINILGGLGNQMFQYAFAYSMAHKTDAVVKLDIEDFSNYDLREYELSLYNISLDLADIDEIDKLKYEQETLFKKVARKLQRTSRPLSSYYYKESGFSYDSHVYELKDNVYFQGYWQSEKYFLDYRDALLKEFLLKDGLHQESRAYEKKINQSVSVSLHIRRGDYVSNAHTNSVHGTCSLEYYKGAVRYLQSNSHPTHFFIFSDDLDWAKENLNFIENITFVSLDKDTPDHEEMYLMSQCKHNIIANSSFSWWGAWLNQNEDKIVVAPKKWFNDTTINTNDLVPKEWIRL

SEQ ID NO: 4 - protein, 300 aa, Helicobacter pylori
MAFKVVQICGGLGNQMFQYAFAKSLQKHLNTPVLLDTTSFDWSNRKMQLELFPIDLPYANAKEIAIAKMQHLPKLVRDALKYIGFDRVSQEIVFEYEPKLLKPSRLTYFFGYFQDPRYFDAISSLIKQTFTLPPPPENNKNNNKKEEEYQRKLSLILAAKNSVFVHIRRGDYVGIGCQLGIDYQKKALEYMAKRVPNMELFVFCEDLKFTQNLDLGYPFTDMTTRDKEEEAYWDMLLMQSCKHGIIANSTYSWWAAYLMENPEKIIIGPKHWLFGHENILCKEWVKIESHFEVKSQKYNA

SEQ ID NO: 5 - protein, 299 aa, Helicobacter pylori
MAFKVVQICGGLGNQMFQYAFAKSLQKHSNTPVLLDITSFDWSNRKMQLELFPIDLPYASEKEIAIAKMQHLPKLVRNVLKCMGFDRVSQEIVFEYEPKLLKTSRLTYFYGYFQDPRYFDAISPLIKQTFTLPPPPENGNNKKKEEEYHRKLALILAAKNSVFVHIRRGDYVGIGCQLGIDYQKKALEYMAKRVPNMELFVFCEDLEFTQNLDLGYPFMDMTTRDKEEEAYWDMLLMQSCKHGIIANSTYSWWAAYLINNPEKIIIGPKHWLFGHENILCKEWVKIESHFEVKSQKYNA

SEQ ID NO: 6 - protein, 283 aa, Bacteroides fragilis
  source 1..283: note = str. S23L17
MKIVQIIGGLGNQMFQFAFYLALKEKYVNVKLDTSSFGAYTHNGFELDKVFHVEYLKASIRERIKLSYQGSEIWIRVLRKLLKRKKTEYVEPYLCFDENAISLSCDKYYIGYWQSYKYFTNIEAAIRGQFHFSKVLSDKNEFIKKQMQNSNSVSLHVRLGDYVNNPAYSNICTSAYYNKAINIIQSKVSEPKFFVFSDDTVWCKDHLKIPNCHIIDWNNKEESYWDMCLMTYCKHNIIANSSFSWWGAWLNTNPERIVIAPGKWINDDRVQVSDIIPSDWICV

SEQ ID NO: 7 - protein, 285 aa, Pedobacter cryoconitis
MKIIRFLGGLGNQMFQYACYKALSKKYPDVKVDLNSFNFDTAHNGYELEDIFQVSTNKVSPFTGGIYDIKNRKWIYRKIRRVLNLKKYHKAEEKDFTYDPAIFSNSKSRYYSGFWQNENYFIDIADEIRKDFKFSPLTAQQNIDTLKKIEQTNSVGVHVRRGDYVNHPAFGGLCEKEYYDQALQIIQSRTEAAKFFLFSNDIDWCVNNLNIKNCEFISWNKGTQSYIDMQLMSACKHNIIANSSFSWWAAWLNNNPEKIVIGPKKWLHGDQYDTSALLPAGWIKI

SEQ ID NO: 8 - protein, 307 aa, Spirochaetes bacterium
MKIVKIIGGLGNQLFQYAFSRALALKTGDQVLLDITSYNEAQSRIHNGFELPGIFPVHYEVANERDVQRLSTQPRSALSKVRRKYFTKRTHYIDKIFRFNPQVFDLKGDWYLEGWWQDARYFDFCSDLIRKELTFIADPGTENRELAAIFEKSRYSLVPVSLHVRRGDALPNPDTWVCTPIYYRHAIEAARQAVRVLGRMARPYFLVFSDDLAWCKANLALEPSEAIYVDWNRGANSWRDMWLMSQCRIHVIANSTFSWWGAWLDQHPDKIVYAPEHWSLAHPRRFAYYHYTFDDVVPAAWKRLPIL

SEQ ID NO: 9 - protein, 357 aa, Otolemur garnettii
MWLARHRHLCLAFLLVCVLSAISFLHFHQDIFRHGLDLSILCPDRHLMTAPVAIFCLEGTPLDPNTSTSCPQHPASLSGTWTIYPDGRFGNQMGQYATLLALAQLNGRPAFILPAMHATLAPVFRITLPVLSPEVDSHTPWQELQLHDWMSEEYAHLSDPFLKLSGFPCSWTFFHHLREQIRREFTLHDHLREKAQSLLSQLRLGLTGDRPRTFVGVHVRRGDYLQVMPQRWRGVVGDQAYLQQAMDWFRARYEAPIFVVTSNGMEWCRENIDTSQGDVFFAGNGQEGAPGQDFALLTQCNHTIMTIGTFGFWAAYLAGGDTVYLANFTLPDSEFLKIFKPKAAFLPEWVGINADLS

SEQ ID NO: 10 - protein, 267 aa, Polaribacter atrinae
MIVVRILGGLGNQMFQYAYARALSLNGYNVKLDLSKIKKYKLHGGYQLDKYNIDLEEADSFSILLGKTGLKGNKKEKSLLFDNNLKLLNGNEYLKGYFQTEKYFKEIRNTLLTEFVIKQKASKEMLSITKQIEAAKNSCSLHIRRGDYISNKKANSVHGTCDLEYYKKAIKVISNKYSNITFFVFSDDISWTKENLLIESVTYIDIKSIPHEDMYLMSLCNHNITANSSFSWWGAWLNKNETKTVIAPKQWYIDKENEIACLNWIKI

SEQ ID NO: 11 - protein, 267 aa, Polaribacter sp.
  source 1..267: note = SA4-10
MITVRIVGGLGNQMFQYAYAKALEQKGYAVKIDTTKFKKYKLHGGYQLDKFNIDLNSTSSLPSILSKIGIVKSIKEKNLLFDKQLTSLRGNKYVKGYFQTEKYFNEIRAVLLNQFIIKNSISEDTNKVAKTIFSSINSCSLHIRRGDYISDKKANSVHGTCALEYYEGAIKIMNDTYKNSTFFVFSDDIPWTKENLKIENAFYVDTKTIPHEDMYLMSLCKNNITANSSFSWWGAWLNKNHTKTVIAPKNWFVNKENEVACENWIKL

SEQ ID NO: 12 - protein, 293 aa, Helicobacter bilis
MGDYKIVELTCGLGNQMFQYAFAKALQKHLQVPVLLDKTWYDTQDNSTQFSLDIFNVDLEYATNTQIEKAKARVSKLPGLLRKMFGLKKHNIAYSQSFDFHDEYLLPNDFTYFSGFFQNAKYLKGLEQELKSIFYYDSNNFSNFGKQRLELILQAKNSIFIHIRRGDYCKIGWELGMDYYKRAIQYIMDRVEEPKFFIFGATDMSFTEQFQKNLGLNENNSANLSEKTITQDNQHEDMFLMCYCKHAILANSSYSFWSAYLNNDANNIVIAPTPWLLDNDNIICDDWIKISSK

SEQ ID NO: 13 - protein, 293 aa, Thermosynechococcus vestitus
MIIVRLCGGLGNQMFQYAAGLAAAHRIGSEVKFDTHWFDATCLHQGLELRRVFGLELPEPSSKDLRKVLGACVHPAVRRLLSRRLLRALRPKSLVIQPHFHYWTGFEHLTDNVYLEGYWQSERYFSNIADIIRQQFRFVEPLDPHNAALMDEMQSGVSVSLHIRRGDYFNNPQMRRVHGVDLSEYYPAAVATMIEKTNAERFYVFSDDPQWVLEHLKLPVSYTVVDHNRGAASYRDMQLMSACRHHIIANSTFSWWGAWLNPRPDKVVIAPRHWFNVDVFDTRDLYCPEWIVL

SEQ ID NO: 14 - protein, 296 aa, Candidatus Nomurabacteria bacterium
MIITRLQGGIGNQMFQYALGRALSVKNNVPLGLDLTFLLDRTPIPNFANFTFRNYDLDVFNIEAVIVSKKDIPFLYRKHNLGIFMRYIDYFRRKLISTPGKEKMNCSFDASILQLGSDAYLEGWWQSYKYFESIEDIIRKDFTFKNKLPLHIENLNEVIKKENSLCVHVRRGDYVGNFHHEVVGKDYYDRGIERIKSLTNIDKIYVFSDDVKWCEGNMKFDLPTMFVGEEYAGTKAEGHMALMSACHNFIIPNSSFSWWGAWLADYKDKVVIVPKQWFVDASINSDDLIPSGWIRI

SEQ ID NO: 15 - protein, 288 aa, Pseudomonas sp.
  source 1..288: note = 1 R 17
MIIAHIIGGLGNQMFQYAAARALSVEKNTGLFLDVTSFESYALHQGFELNKIFSADFKTASYSDIQKILGWQAPAVVRSILHRPRLAWLRKTTLSIEPSFQYWRGVQDLSDNTYLSGYWQSERYFKNIESIIRKDFSFKLPMDNENSRIANLISNTEAVSLHIRRGDYVNNSAYSACSLDYYHAAISHFTHMNKPPTFFIFSDDINWVKEHLKIEHPHCYVDHNNGAASYNDMRLMSLCKHNIIANSSFSWWGAWLNANNDKIVITPKSWFNTNNHIDDLIPPTWISL

SEQ ID NO: 16 - protein, 283 aa, Pseudosulfitobacter pseudonitzschiae
MIFSRLHGRLGNQMFQYAAARALAEHHCTRVVLDDRTALHKDEGSLLRVFDLPDLAQAPLPPAKHERPLAYAAWRALGLRPRIRRENGLGYDRAFTQWSDDSYLHGYWQSERYFAAIAQDIRSAFAFRTPMSAQNTEMAARIASGPSVALHVRRGDYVAVNAMALCDQAYYDAALTSVRKRMENDPTVFVFSDDPAWAKENLPLPFEKVVVDFNGPDADYEDLRLMSQCQHNIIANSSFSWWAAWLGETPDSIVAGPAQWFADTAMSNPDILPARWISVDTSG

SEQ ID NO: 17 - protein, 283 aa, Pedobacter heparinus
MKIIRFLGGLGNQMFQYAFYKSLQHRFPHVKADLQGYQEYTLHNGFELEHIFNIKVNSVSSFTSDLFYNKKWLYRKLRRILNLRNTYIEEKKLFSFDPSLLNNPKSAYYWGYWQNFQYFEHIADDLRKDFQFRAPLSAQNQEVLDQTKLSNSISLHIRRGDYIKDPLLGGLCGPEYYQTAINYITSKVNAARFFIFSDDIDWCIANLKLQDCSFISWNKGTSSYIDMQLMSSCKHHIVANSSFSWWAAWLNPNPDKIVIAPEKWTNDKDINVRMSFPQGWISL

SEQ ID NO: 18 - protein, 311 aa, Rhodopirellula europaea
MIVTRLIGGLGNQLFQYAFGHSLARSTYQTLLIDDSAFIDYRLHPLAIDHFTISASRLSDADRSRVPGKFLRTPVGRALDKVSRFVPGYQGVLPVRREKPFGFRESLLARESDLYLDGYWQSEKFFPGLRGSLREEFQLREQPSETTRRLSAQMKSENSVAIHVRRGDYVTSAKAKQIYRTLDADYYRRCLLDLAAHETDLKLYLFSNDVPWCESNLDVGIPFTPVQHTDGATAHEDLHLIAQCRHVVIANSTFSWWGAYLGQLHPTRRVYYPEPWFHPGTLDGSAMGCDDWISEASLEEQSSLKSSRRAA

SEQ ID NO: 19 - protein, 290 aa, Chromobacterium vaccinii
MIVTRLCGGLGNQLFQYAAARMLAQIHKAKIFVDLGWFENIPNKNTSRYYELDHYRLPIEKLVLESSIQKKMLEAPFFNLIPIKRLGLKIYREQSYCFDTNFYNALDNSYLRGYWQSHLYFTDIRDILVKELQPVTPPSPMDVAIMDMIENSENSISIHVRRGDYVSLKSASNTHGTCSLDYYKKSINFFAEHVSDPHFFVFSDDINWCRENLSFPHKSTFVSHNNAATAFQDLRLMAHCKHNVIANSSFSWWGAWLNSNDHKIVIAPMNWFNQASHDTKDLLPLNWVRL

SEQ ID NO: 20 - protein, 300 aa, Gramella forsetii
MSNKNPVIVEIMGGLGNQMFQFAVAKLLAEKNSSVLLVDTNFYKEISQNLKDFPRYFSLGIFDISYKMGTENGMVNFKNLSFKNRVSRKLGLNYPKIFKEKSYRFDADLFNKKTPIYLKGYFQSYKYFIGVESKIRQWFEFPYENLGVGNEEIKSKILEKTSVSVHIRRGDYVENKKTKEFHGNCSLEYYKNAITYFLDIVKEFNIVFFSDDISWVRDEFKDLPNEKVFVTGNLHENSWKDMYLMSLCDHNIIANSSFSWWAAWLNNNSEKNVIAPKKWFADIDQEQKSLDLLPPSWIRM

SEQ ID NO: 21 - protein, 416 aa, Bos taurus
MPGPAPDARAWPDSKHPQTPEWKREKSTDRSIRIQHGSCKLDLSVHEKMRLLGRPAMWAPGHRHLCLIFLLTCVFACVFFLLIHQNLFHSGLDLFLLCPDRSRVRSPVAILCLSGTPMNPNATFTCPRHSASVSGTWTIDPKGRFGNQMGQYATLLALAQLNGRQAFIQPSMHAVLAPVFRITLPVLAPEVDRHAPWQELELHDWMSEEYAHLKEPWLKLTGFPCSWTFFHHLRDQIRSEFTLHEHLRQEAQRSLSGLRFPRTGGRPSTFVGVHVRRGDYLQVMPLHWKGVVGDRAYLQQAMDWFRARHKAPIFVVTSNGMKWCRENIDTSRGDVIFAGDGQEGAPNKDFALLTQCNHTIMTIGTFGFWAAYLAGGDTIYLANFTLPDSSFLKIFKPEAAFLPEWVGINADLSPLQ

SEQ ID NO: 22 - protein, 308 aa, Acetobacter sp.
  source 1..308: note = CAG:267
MAVSPQESKYSAHVSPDKPLRIVRLGGGLGNQMFQYAFGLAAGDVLWDNTSFLTNHYRSFDLGLYNISGDFASNEQIKKCKNEIRFKNILPRSIRKKFNLGKFIYLKTNRVCERQINRYEPELLSKDGDVYYDGVFQTEKYFKPLRERLLHDFTLTKPLDAANLDMLAKIRAADAVAVHIRRGDYLNPRSPFTYLDKDYFLNAMDYIGKRVDKPHFFIFSSDTDWVRTNIQTAYPQTIVEINDEKHGYFDLELMRNCRHNIIANSTFSWWGAWLNTNPDKIVVAPKQWFRPDAAEYSGDIVPNDWIKL

SEQ ID NO: 23 - protein, 297 aa, unidentified
  source 1..297: note = Parcubacteria group bacterium
MIIVKIEGGLGNQLFQYAFARGISSRLNTEFKIDKSPFDIYYKYHKYALDNFNLKGSLAKDSDFFGFMWLKKQHKLFTFFYNHLRFRKKLLPFYFREQAFHFDSSVFSKDNTYFEGYWQTEKYFRELESELQEEITLNKPLSDYSKGILNQIKSSVAISLHVRRGDYVTGSTISNVPLIHGTCSMDYYKSAIAYISERISNPHFFIFSDDYDWSVENFKSLKYPTVCIKNGADKNYEDLILMASCKHNIIANSSFSWWGAWLNRNKEKIVIAPKKWFNMPKQGTNTDDIIPDTWIKL

SEQ ID NO: 24 - protein, 312 aa, Eisenbergiella tayi
MCCRRIKARNEREVISQLFSEEYIFIMIIIEISGGLGNQMFQYALGQKFISMGKKVKYDLSFYNERVQTLREFELDIFHIDCPVATSNELFYFGKGFSLASRFKQRIGWDKRNVYEEDLDLGYQPQIFGLDNIYLSGYWQSEKYFENIRQRILELYTFSGKLGYENKRFLDKIENSNSVSLHVRRGDYLNEENVKIYGGICTINYYKNAIKYISDRFEKPVFFVFTNDLEWVKNELDIPNKVIVDCNSGSLSYWDMYLMSKCKANIVANSSFSWWGAWLNQHSNRVVVSPRRWFNNHEQTSTLCDDWVRCGG

SEQ ID NO: 25 - protein, 360 aa, Bos taurus
MWAPGHRHLCLIFLLTCVFACVFFLLIHQNLFHSGLDLFLLCPDRSRVRSPVAILCLSGTPMNPNATFTCPRHSASVSGTWTIDPKGRFGNQMGQYATLLALAQLNGRQAFIQPSMHAVLAPVFRITLPVLAPEVDRHAPWQELELHDWMSEEYAHLKEPWLKLTGFPCSWTFFHHLRDQIRSEFTLHEHLRQEAQRSLSGLRFPRTGGRPSTFVGVHVRRGDYLQVMPLHWKGVVGDRAYLQQAMDWFRARHKAPIFVVTSNGMKWCRENIDTSRGDVIFAGDGQEGAPNKDFALLTQCNHTIMTIGTFGFWAAYLAGGDTIYLANFTLPDSSFLKIFKPEAAFLPEWVGINADLSPLQ

SEQ ID NO: 26 - protein, 288 aa, Pseudoalteromonas distincta
MIKVKAIGGLGNQLFQYATARAIAEKRGDGVVVDMSDFSSYKTHPFCLNKFRCKATYESKPKLINKLLSNEKIRNLLQKLGFIKKYYFETQLPFNEDVLLNNSINYLTGYFQSEKYFLSIRECLLDELTLIEDLNIAETAVSKAIKNAKNSISIHIRRGDYVSNEGANKTHGVCDSDYFKKALNYFSERKLLDEHTELFIFSDDIEWCRNNLSFDYKMNFVDGSSERPEVDMVLMSQCKHQVISNSTFSWWGAWLNKNDEKVVVAPKEWFKSTDLDSTDIVPNQWIKL

SEQ ID NO: 27 - protein, 288 aa, Cupriavidus sp.
  source 1..288: note = GA3-3
MIVTRVIGGLGNQMFQYAAGRALARRLGVPLKIDSSGFADYPLHNYGLHHFALKAVQAGDREIPSGRAENRWAKALRRFGLGTELRVFRERGFAVDPEVMKLPDGTYLDGYWQSESYFAEMTQELRRDFQIATPPTSENAEWLARIGGDEGAVSIHVRRGDYVTNASANAVHGICSLDYYMRAARYVAENIGVKPTFYVFSDDPDWVAGNLHLGHETRYVRHNDSARNYEDLRLMSACRHHIIANSTFSWWGAWLNASEKKVVIAPAQWFRDEKYDTRDLLPPTWTKL

SEQ ID NO: 28 - protein, 288 aa, unidentified
  source 1..288: note = Microgenomates group bacterium
MIIVRLKGGLGNQMFQYATGLAVASRRGQELKLDSTGYDDPRVINSDIPRKYALYAFSISGSIAVRDEVGKARNPYGVFSKAVRFFNQKVLRKYYADYDPAFFKKNNKYIEGYFQSEKNFGNIKEKVVKEFTLKKEFESEFFLTEKNKIDRTKSVSVHIRRGDYVYDPKINSVHGVCSREYYERAINLMKSKIEAPAFYFFSDDIEWVKKEFGGHSDFRYISNPNLKEYEELILMSLCAHNIIANSSFSWWGAYLNQNPNKIVIAPKKWMNMEPDPHPNIIPEWWMRI

SEQ ID NO: 29 - protein, 273 aa, Algibacter lectus
MVIVKLIGGLGNQMFQYAAAKALALHTKQELRLDLSGFDDYKLRAFDLHHFNINAKPFRQKSKWIRKLENKLKLTTYYNEQSFRFNPEVFNIITKNILLQGYFQAEDYFITYRNDILNDFKIVSPLKKQTQNLLVEMSKTNAVSIHIRRGDFLTHEVHNTSKEEYYREAMIVIENKIEQPTYYVFSDDMDWVKANFKTKYNTVYVDFNDASTAFEDIKLMSNCQHNIIANSSFSWWSAWLNTNPNKIVIAPKQWFNGEQYDYTDVVPKRWIKL

SEQ ID NO: 30 - protein, 295 aa, Geotalea uraniireducens
MIIARLQGGLGNQMFQYAVGLHLALTHNVELKIDITMFSDYKWHTYSLRPFNIRESIATEEEIKALTDVKMDRPYKKIDNFLCRLLRKSQKISATHVKEKHFHYDPDILKLPDNVYLDGYWQSEKYFKEIENIIRQTFIIKNPQLGRDKELACKILSTESVCLHIRRGNYVTDKTTNSVLGPCDLSYYSNCIKSLAGNNKDPHFFVFSNDHEWVSKNLKLDYPTIYVDHNNEDKDYEDLRLMSQCKHHIIANSTFSWWSAWLCSNPDKVIYAPQKWFRVDEYNTKDLLPSNWLIL

SEQ ID NO: 31 - protein, 365 aa, Equus caballus
MWAPSRRHLCLIFLLVCVLSSIAFLYVHQGLFHDGVDLFALCPSHHLGTPHVAIFCLSGTTMTSNASLSCPQQPASLTGTWTIHPDGRFGNQMGQYATLLALAQLNGRQAFILPAMHATLAPVFRITLPVLSPQVDSQTSWLKLQLHDWMSEEYARVEHPVLKLTGFPCSWTFFHHIREQIRSQFTLHDHLRQDAQGFLSQLRLGRTGGRPSTFVGVHVRRGDYLQVMPQLWKGVVGDRAYLQQAMDWFRARHEAPIFVVTSNGMDWCRQNIDTSRGDVIFAGNGLEASPGKDFALLTQCNHTIMTIGTFGFWAAYLAGGDTVYLANFTLPDSNFLKIFKPEAAFLPEWVGINADLSPLRTPAGR

SEQ ID NO: 32 - protein, 361 aa, Camelus ferus
MWAPSRRHLCLTFLLVCVSAAILFFHIHQDLLHDALDLSALCPDYNLVTSPVAIFCLSGTPINPNASISCPKHPASSSGTWTIYPDGRFGNQMGQYATLLALAQLNGRQAFIQPAMHAALAPMFRITLPVLAPEVDRHAPWRELELHDWMSEEYAHLEEPWLKLTGFPCSWTFFHHLREQILREFTLHDYLRQEAQRLLSRLRLRRTGKRPSTFVGVHVRRGDYLEVMPHRWKGVVGDRSYLQQAMDWFRARHEAPIFVVTSNGMGWCRKNIDTSQGDVIFAGNGQEDAPGKDFALLVQCNHTIMTIGTFGFWAAYLAGGDTVYLANFTLPNSKFLKIFKPKAAFLPEWVGINADLSPLHM

SEQ ID NO: 33 - protein, 285 aa, unidentified
  source 1..285: note = Parcubacteria group bacterium
MKIHGGLGNQMFQYALGRNLSLIHKVPVKIDYSYLKTENQSGRRFELDGFRIQAVEATENDIRRYGSTFQKMVDRIRPEAKRKKITEQGDGFHSDVLERFDAYFDGHWQNERYFKAHEKTIREDFSLKNRFGPASEAMARKIQSEKNPTSVHIRRGDYVSIEKIADTHGTLPVSYYRTACDKILEKLPDARFFVSSDDIDWAKENFPREYPATFISAPEITDCEELTLMSLCKHNIIANSTFSWWGAWLNTNPEKIVIAPKLWFVNPNRVPKDLIPSSWIPLEAY

SEQ ID NO: 34 - protein, 277 aa, unidentified
  source 1..277: note = Candidatus Magasanikbacteria bacterium
MIFDKLSGGLGNQMFQYAAGYSLSLNNRIPLNLDLSSFEHKKTGITHRYFLLNKFNIDRNILINHMEKISGYRKFLSKFITKFFGENFYYNITFLSSKYLDGYFQSEKYFKNIEDILRKEFTLKNEMSVVARQVESKISNSINSVSLHIRRGDYVLDNKTNSYHGICDLDYYKKAVEYFKNKLGELNIFVFSDDIVWVKENLRFENLYFVSSPDIKDYEELILMSRCKHNIIANSSFSWWGAWLNANKNKTVITPKKWFQKYNINQKHIVPKSWIRL

SEQ ID NO: 35 - protein, 287 aa, Runella slithyformis
MIIVKLSGGLGNQLFQYAFGRHLATVNQKELKLDTSALTKTSDWTNRSYALDAFNIRAQEATPEEIKALAGKPNRLLQRVGRKVGITPIQYFQEPHFHFYSSALSIKSSHYLEGYWQSEKYFEAITPILREEFAFTISPSTHAQTIKEKISNGTSVSIHLRRGDYVKTSKANRYLRPLTMDYYQKAIDYINQRVKNPNFFLFSDDIKWAKSQVTFPPTTHFSTGTSAHEDLWLMTHCRHHIIANSTFSWWGAWLNQQPDKIVIAPQKWFSTERFDTKDLLPEPWIQL

SEQ ID NO: 36 - protein, 416 aa, Bos taurus
MPGPAPDARAWPDSKHPQTPEWKREKSTDRSIRIQHGSCKLDLSVHEKMRLLGRPAMWAPGHRHLCLIFLLTCVFACVFFLLIHQNLFHSGLDLFLLCPDRSRVRSPVAILCLSGTPMNPNATFTCPRHSASVSGTWTIDPKGRFGNQMGQYATLLALAQLNGRQAFIQPSMHAVLAPVFRITLPVLAPEVDRHAPWQELELHDWMSEEYAHLKEPWLKLTGFPCSWTFFHHLRDQIRSEFTLHEHLRQEAQRSLSGLRFPRTGGRPSTFVGVHVRRGDYLQVMPLHWKGVVGDRAYLQQAMDWFRARHKAPIFVVTSNGMKWCRENIDTSRGDVIFAGDGQEGAPNKDFALPTQCNHTIMTIGTFGFWAAYLAGGDTIYLANFTLPDSSFLKIFKPEAAFLPEWVGINADLSPLQ

SEQ ID NO: 37 - protein, 357 aa, Ovis aries
MWSALAAGALHSSPSRLWAATRQELSGLDLFLLCPDRSRVTSPVAILCLSGTPVNTNATFSCPKHPASISGTWTIDPKGRFGNQMGQYATLLALAQLNGRQAFIQPSMHAILAPVFRITLPVLAPEVDRHAPWQELELHDWMSEEYAHLKEPWLKLTGFPCSWTFFHHLREQIRSEFTLHEHLRQEAQRSLSGLRFPRTGDRPSTFVGVHVRRGDYLQVMPLHWKGVVGDHAYLQQAMDWFRARHKAPIFVVTSNGMEWCRENIDTSRGDVIFAGDGQEGAPHKDFALLTQCNHTIMTIGTFGFWAAYLAGGDTVYLANFTLPDSSFLKIFKPEAAFLPEWVGINADLSPLQGKAES

SEQ ID NO: 38 - protein, 291 aa, Phocaeicola vulgatus
MQITKKGQRNMRLIKVTGGLGNQMFIYAFYLRMKKYYPKVRIDLSDMMHYKVHYGYEMHRVFNLPHTEFCINQPLKKVIEFLFFKKIYERKQAPNSLRAFEKKYFWPLLYFKGFYQSERFFADIKDEVRESFTFDKNKANSRSLNMLEILDKDENAVSLHIRRGDYLQPKHWATTGSVCQLPYYQNAIAEMSRRVASPSYYIFSDDIAWVKENLPLQNAVYIDWNTDEDSWQDMMLMSHCKHHIICNSTFSWWGAWLNPNMDKTVIVPSRWFQHSEAPDIYPTGWIKVPVS

SEQ ID NO: 39 - protein, 301 aa, Moorena bouillonii
MKGKCMGVVIVRLSGGLGNQMFQYAIGRKIALVNNVQLKLDISSFEHDLLRMYNLYWFQIKQAFASSEELAALKSLRQTKESNPVIIRLRQVMKRFASWKVFREEQLMPFNPNIMTSSDKIYLDGYWQSEKYFLDIEDVIRREYTCKYEPNAQSKKIAEMIANSHSVSIHVRRGDYVSNPANNQLHGTCSLTYYQQCVEQIAKEVLHPHFFVFSDHPIWVKENLCLDYPMTFVTHNNHLRDYEDLWLMSHCQHHIIANSSFSWWAAWLNPNLNKKVFAPKKWFNDPRLDTRDLLPDNWIKV

SEQ ID NO: 40 - protein, 291 aa, Dysgonomonas mossii
MVTVLLSGGLGNQMFQYAAAKSLAIRLNTALSVDLYTFSKKTQATVRPYELGIFNIEDVVETSSLKAKAVIKARPFIQRHRSFFQRFGVFTDTYAILYQPTFEALTGGVIMSGYFQNESYFKNISELLRKDFSFKYPLIGENKDVAGQISENQSVAVHIRRGDYLNKNSQSNFAILEKDYYEKAINYISAHVKNPEFYVFSEDFDWIKDNLNFKEFPVTFIDWNKGKDSYIDMQLMSLCKHNIIANSSFSWWSAWLNNSEERKIVAPERWFVDEQKNELLDCFYPQGWIKI

SEQ ID NO: 41 - protein, 294 aa, Colwellia psychrerythraea
MKVVRVCGGFGNQLFQYAFYLAVKHKFNETTKLDIHDMASYELHNGYELERIFNLNENYCSAEEKLAVQSTKNIFTKLLKEIKKYTPFIPRTYIKEKKHLHFSYQEVDLGTKDTSIYYRGSWQNPQYFNSIASEIREKLTFPEFTEPKSLALHQEISEHETVAVHIRRGDYLKHKALGGICDLPYYQNAIKEIEGLVEKPLFVIFSDDITWCRANINVEKVRFVDWNSGEQSFQDMHLMSLCTHNIIANSSFSWWGAWLNANPNKIVISPNKWIHYTDSMGIVPSEWIKVETSI

SEQ ID NO: 42 - protein, 373 aa, Escherichia coli [sequence omitted]

SEQ ID NO: 43 - protein, 377 aa, Caenorhabditis briggsae
MEGLEACIGQSHEVMTTPAAELAAFRARKVALITGISGQDGSYLAELLLSKGYKVHGIIRRSSSFNTARIEHLYSNPMTHNGDSSFSLHYGDMTDSSCLIKLISTIEPTEVYHLAAQSHVKVSFDLPEYTAEVDAVGTLRLLDAIHACRLTEKVRFYQASTSELYGKVQEIPQSEKTPFYPRSPYAVAKMYGYWIVVNYREAYKMFACNGILFNHESPRRGETFVTRKITRSVAKISLGQQESIELGNLSALRDWGHAREYVEAMWRILQHDAPDDFVIATGKQFSVREFCNLAFAEIGEVLEWEGEGVEEVGKNKDGIVRVKVSPKYYRPTEVETLLGNPEKAKKTLGWEAKVTVPELVKEMVASDIALMKANPMA

SEQ ID NO: 44 - protein, 382 aa, Caenorhabditis elegans
MEARNAEGLESCIEKIQEVKLSSFAELKAFRERKVALITGITGQDGSYLAELLLSKGYKVHGIIRRSSSFNTARIEHLYGNPVTHNGSASFSLHYGDMTDSSCLIKLISTIEPTEIYHLAAQSHVKVSFDLPEYTAEVDAVGTLRLLDAIHACRLTEKVRFYQASTSELYGKVQEIPQSELTPFYPRSPYAVAKMYGYWIVVNYREAYKMFACNGILFNHESPRRGETFVTRKITRSVAKISLRQQEHIELGNLSALRDWGHAKEYVEAMWRILQQDTPDDFVIATGKQFSVREFCNLAFAEIGEQLVWEGEGVDEVGKNQDGVVRVKVSPKYYRPTEVETLLGNPAKARKTLGWEPKITVPELVKEMVASDIALMEADPMA

SEQ ID NO: 45 - protein, 373 aa, Arabidopsis thaliana
MASENNGSRSDSESITAPKADSTVVEPRKIALITGITGQDGSYLTEFLLGKGYEVHGLIRRSSNFNTQRINHIYIDPHNVNKALMKLHYADLTDASSLRRWIDVIKPDEVYNLAAQSHVAVSFEIPDYTADVVATGALRLLEAVRSHTIDSGRTVKYYQAGSSEMFGSTPPPQSETTPFHPRSPYAASKCAAHWYTVNYREAYGLFACNGILFNHESPRRGENFVTRKITRALGRIKVGLQTKLFLGNLQASRDWGFAGDYVEAMWLMLQQEKPDDYVVATEEGHTVEEFLDVSFGYLGLNWKDYVEIDQRYFRPAEVDNLQGDASKAKEVLGWKPQVGFEKLVKMMVDEDLELAKREKVLVDAGYMDAKQQP

SEQ ID NO: 46 - protein, 372 aa, Homo sapiens
MAHAPARCPSARGSGDGEMGKPRNVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAVKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFLHIGKTIVWEGKNENEVGRCKETGKVHVTVDLKYYRPTEVDFLQGDCTKAKQKLNWKPRVAFDELVREMVHADVELMRTNPNA

SEQ ID NO: 47 - protein, 399 aa, Caenorhabditis elegans
MPTGKSESSDISEVVGNMEISKVEGLEACIGMSHEVSTTPAAELAAFRARKVALITGISGQDGSYLAELLLSKGYKVHGIIRRSSSFNTARIEHLYSNPITHHGDSSFSLHYGDMTDSSCLIKLISTIEPTEVYHLAAQSHVKVSFDLPEYTAEVDAVGTLRLLDAIHACRLTEKVRFYQASTSELYGKVQEIPQSEKTPFYPRSPYAVAKMYGYWIVVNYREAYNMFACNGILFNHESPRRGETFVTRKITRSVAKISLGQQESIELGNLSALRDWGHAREYVEAMWRILQHDSPDDFVIATGKQFSVREFCNLAFAEIGEVLQWEGEGVEEVGKNKDGVIRVKVSPKYYRPTEVETLLGNAEKAKKTLGWEAKVTVPELVKEMVASDIILMKSNPMA

SEQ ID NO: 48 - protein, 372 aa, Cricetulus griseus
MAHAPASCPSSRNSGDGDKGKPRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKIHVTVDLKYYRPTEVDFLQGDCSKAQQKLNWKPRVAFDELVREMVQADVELMRTNPNA

SEQ ID NO: 49 - protein, 356 aa, Dictyostelium discoideum
MSEERKVALITGITGQDGSYLTEFLISKGYYVHGIIQKIFHHFNTIVKNIYIKIDMLKEKESLTLHYGDLTDASNLHSIVSKVNPTEIYNLGAQSHVKVSFDMSEYTGDVDGLGCLRLLDAIRSCGMEKKVKYYQASTSELYGKVQEIPQSETTPFYPRSPYAVAKQYAYWIVVNYREAYDMYACNGILFNHESPRRGPTFVTRKITRFVAGIACGRDEILYLGNINAKRDWGHARDYVEAMWLMLQQEKPEDFVIATGETHSVREFVEKSFKEIDIIIKWRGEAEKEEGYCEKTGKVYVKIDEKYYRPTEVDLLLGNPNKAKKLLQWQIKTSFGELVKEMVAKDIEYIKNGDKYN

SEQ ID NO: 50 - protein, 351 aa, Sinorhizobium fredii
MTDRKVALISGVTGQDGAYLAELLLDEGYIVHGIKRRSSSFNTQRIEHIYQERHDPEARFFLHYGDMTDSTNLLRIVQQTQPHEIYNLAAQSHVQVSFETPEYTANADAIGTLRMLEAIRILGLTNRTRFYQASTSELYGLAQESPQNEKTPFYPRSPYAAAKLYAYWIVVNYREAYGMHASNGILFNHESPLRGETFVTRKITRAAAAISLGKQEVLYLGNLDAQRDWGHAREYVRGMWMMCQQDRPGDYVLATGVTTSVRTFVEWAFEETGMTIEWVGEGIEERGIDAATGRCVVAVDPRYFRPTEVDLLLGDATKARQVLGWRHETSVRDLACEMVREDLSYLRGTRQ

SEQ ID NO: 51 - protein, 361 aa, Arabidopsis thaliana
MASRSLNGDSDIVKPRKIALVTGITGQDGSYLTEFLLEKGYEVHGLIRRSSNFNTQRLNHIYVDPHNVNKALMKLHYGDLSDASSLRRWLDVIKPDEVYNLAAQSHVAVSFEIPDYTADVVATGALRLLEAVRSHNIDNGRAIKYYQAGSSEMFGSTPPPQSETTPFHPRSPYAASKCAAHWYTVNYREAYGLYACNGILFNHESPRRGENFVTRKITRALGRIKVGLQTKLFLGNIQASRDWGFAGDYVEAMWLMLQQEKPDDYVVATEESHTVKEFLDVSFGYVGLNWKDHVEIDKRYFRPTEVDNLKGDASKAKEMLGWKPKVGFEKLVKMMVDEDLELAKREKVLADAGYMDAQQQP

SEQ ID NO: 52 - protein, 351 aa, Rhizobium fredii
MTDRKVALISGVTGQDGAYLAELLLDEGYIVHGIKRRSSSFNTQRIEHIYQERHDPEARFFLHYGDMTDSTNLLRIVQQTQPHEIYNLAAQSHVQVSFETPEYTANADAIGTLRMLEAIRILGLIHRTRFYQASTSELYGLAQEIPQNEKTPFYPRSPYAAAKLYAYWIVVNYREAYGMHASNGILFNHESPLRGETFVTRKITRAAAAISLGKQEVLYLGNLDAQRDWGHAREYVRGMWMMCQQDRPGDYVLATGVTTSVRTFVEWAFEETGMTIEWVGEGIEERGIDAATGKCVVAVDPRYFRPTEVDLLLGDATKARQVLGWRHETSVRDLACEMVREDLSYLRGTRQ

SEQ ID NO: 53 - protein, 372 aa, Mus musculus
MAQAPAKCPSYPGSGDGEMGKLRKVALITGITGQDGSYLAEFLLEKGYEVHGIVRRSSSFNTGRIEHLYKNPQAHIEGNMKLHYGDLTDSTCLVKIINEVKPTEIYNLGAQSHVKISFDLAEYTADVDGVGTLRLLDAIKTCGLINSVKFYQASTSELYGKVQEIPQKETTPFYPRSPYGAAKLYAYWIVVNFREAYNLFAVNGILFNHESPRRGANFVTRKISRSVAKIYLGQLECFSLGNLDAKRDWGHAKDYVEAMWLMLQNDEPEDFVIATGEVHSVREFVEKSFMHIGKTIVWEGKNENEVGRCKETGKVHVTVDLKYYRPTEVDFLQGDCSKAQQKLNWKPRVAFDELVREMVQADVELMRTNPNA

SEQ ID NO: 54 - protein, 361 aa, Arabidopsis thaliana; identical to SEQ ID NO: 51

SEQ ID NO: 55 - protein, 399 aa, Caenorhabditis elegans; identical to SEQ ID NO: 47

SEQ ID NO: 56 - protein, 356 aa, Dictyostelium discoideum; identical to SEQ ID NO: 49

SEQ ID NO: 57 - protein, 395 aa, Drosophila melanogaster
MLNTRLIAMSTSDGAPETKKQRPESSSNGSKDQNGTEAGAEGDSRDKVALITGITGQDGSYLAEFLLKKDYEVHGIIRRASTFNTTRIEHLYADPKAHKGGRMKLHYGDMTDSSSLVKIINMVKPTEIYNLAAQSHVKVSFDLSEYTAEVDAVGTLRILDAIRTCGMEKNVRFYQASTSELYGKVVETPQNEQTPFYPRSPYACAKMYGFWIVINYREAYNMYACNGILFNHESPRRGENFVTRKITRSVAKIYHKQMEYFELGNLDSKRDWGHASDYVEAMWMMLQRESPSDYVIATGETHSVREFVEAAFKHIDREITWKGKGVDEVGVENGTGIVRVRINPKYFRPTEVDLLQGDASKANRELNWTPKVTFVELVSDMMKADIELMRKNPIA

SEQ ID NO: 58 - protein, 395 aa, Drosophila melanogaster; identical to SEQ ID NO: 57

SEQ ID NO: 59 - protein, 382 aa, Caenorhabditis elegans; identical to SEQ ID NO: 44

SEQ ID NO: 60 - protein, 377 aa, Caenorhabditis briggsae; identical to SEQ ID NO: 43

SEQ ID NO: 61 - protein, 372 aa, Mus musculus; identical to SEQ ID NO: 53

SEQ ID NO: 62 - protein, 372 aa, Cricetulus griseus; identical to SEQ ID NO: 48

SEQ ID NO: 63 - protein, 351 aa, Rhizobium fredii; identical to SEQ ID NO: 52

SEQ ID NO: 64 - protein, 351 aa, Sinorhizobium fredii; identical to SEQ ID NO: 50

SEQ ID NO: 65 - protein, 587 aa, Kluyveromyces lactis
MADHSSSSSSLQKKPINTIEHKDTLGNDRDHKEALNSDNDNTSGLKINGVPIEDAREEVLLPGYLSKQYYKLYGLCFITYLCATMQGYDGALMGSIYTEDAYLKYYHLDINSSSGTGLVFSIFNVGQICGAFFVPLMDWKGRKPAILIGCLGVVIGAIISSLTTTKSALIGGRWFVAFFATIANAAAPTYCAEVAPAHLRGKVAGLYNTLWSVGSIVAAFSTYGTNKNFPNSSKAFKIPLYLQMMFPGLVCIFGWLIPESPRWLVGVGREEEAREFIIKYHLNGDRTHPLLDMEMAEIIESFHGTDLSNPLEMLDVRSLFRTRSDRYRAMLVILMAWFGQFSGNNVCSYYLPTMLRNVGMKSVSLNVLMNGVYSIVTWISSICGAFFIDKIGRREGFLGSISGAALALTGLSICTARYEKTKKKSASNGALVFIYLFGGIFSFAFTPMQSMYSTEVSTNLTRSKAQLLNFVVSGVAQFVNQFATPKAMKNIKYWFYVFYVFFDIFEFIVIYFFFVETKGRSLEELEVVFEAPNPRKASVDQAFLAQVRATLVQRNDVRVANAQNLKEQEPLKSDADHVEKLSEAESV

SEQ ID NO: 66 - protein, 579 aa, Neurospora crassa
MSSHGSHDGASTEKHLATHDIAPTHDAIKIVPKGHGQTATKPGAQEKEVRNAALFAAIKESNIKPWSKESIHLYFAIFVAFCCACANGYDGSLMTGIIAMDKFQNQFHTGDTGPKVSVIFSLYTVGAMVGAPFAAILSDRFGRKKGMFIGGIFIIVGSIIVASSSKLAQFVVGRFVLGLGIAIMTVAAPAYSIEIAPPHWRGRCTGFYNCGWFGGSIPAACITYGCYFIKSNWSWRIPLILQAFTCLIVMSSVFFLPESPRFLFANGRDAEAVAFLVKYHGNGDPNSKLVLLETEEMRDGIRTDGVDKVWWDYRPLFMTHSGRWRMAQVLMISIFGQFSGNGLGYFNTVIFKNIGVTSTSQQLAYNILNSVISAIGALTAVSMTDRMPRRAVLIIGTFMCAAALATNSGLSATLDKQTQRGTQINLNQGMNEQDAKDNAYLHVDSNYAKGALAAYFLFNVIFSFTYTPLQGVIPTEALETTIRGKGLALSGFIVNAMGFINQFAGPIALHNIGYKYIFVFVGWDLIETVAWYFFGVESQGRTLEQLEWVYDQPNPVKASLKVEKVVVQADGHVSEAIVA

SEQ ID NO: 67 - protein, 579 aa, Neurospora crassa; identical to SEQ ID NO: 66

SEQ ID NO: 68 - protein, 525 aa, Neurospora crassa
MGIFNKKPVAQAVDLNQIQEEAPQFERVDWKKDPGLRKLYFYAFILCIASATTGYDGMFFNSVQNFETWIKYFGDPRGSELGLLGALYQIGSIGSIPFVPLLTDNFGRKTPIIIGCVIMIVGAVLQATAKNLDTFMGGRTMLGFGNSLAQIASPMLLTELAHPQHRARLTTIYNCLWNVGALVVSWLAFGTNYINNDWSWRIPALLQAFPSIIQLLGIWWVPESPRFLIAKDKHDEALHILAKYHANGDPNHPTVQFEFREIKETIRLEMESTKNSSYLDFFKSRGNRYRLAILLSLGFFSQWSGNAIISNYSSKLYETAGVTDSTAKLGLSAGQTGLALIVSVTMALLVDKLGRRLAFLASTGGMCGTFVIWTLTAGLYGEHRLKGADKAMIFFIWVFGIFYSLAWSGLLVGYAIEILPYRLRGKGLMVMNMSVQCALTLNTYANPVAFDYFGPDHSWKLYLIYTCWIAAEFVFVFFMYVETKGPTLEELAKVIDGDEADVAHIDIHQVEKEVEIHEHEGKSVA

SEQ ID NO: 69 - protein, 525 aa, Neurospora crassa; identical to SEQ ID NO: 68

SEQ ID NO: 70 - protein, 539 aa, Emericella nidulans
MERRTYGFETTISRDADKGVFSVNNAALHMATLRVKPRLLTKRMLKLYWCIGVAMLNSCINGYNGSLMGSINSYRQYREYFGFDLEEGTSTTGIVYAIYTIGNIVGSFFAGPFTDFRGRRMGMAIGALWIIAGTIVQATCHNLGGFMAGRFLLGFGVATSATAGPAYVSEMAHPAYRGAMTGLYNVLWFGGGIPGTFIPWRTSTIDGTQSWRIPVWLQMVFSGLVLLLCFTIPESPRWLISCDRHEAAIRVLAEYHGEGDRNSPLVQLEYREMLEDISNVGADKRWWDYRELFDSRETRYRSMLVVFMAFFGQWSGNGPVSYYYPQMLAGAGISSNHTRLLLQGLQNIVQFTGAIFGALITDRVGRRPQLLVSTSIIVFLFVIITALNATNVQVAGDGGGVVAKSSVTARAQIAMIFIFGFVYSAGWTPNQAMYPVECLRYESRAKGMGMNNFFINIASFYNTFVTGIAFTRIGWKYYFLFIFWCTFEVLIIYFLFVETSKRTLEELTVIFQQKRPVQASLDKEEIFVSGDEIVEVRSW

SEQ ID NO: 71 - protein, 539 aa, Aspergillus nidulans; identical to SEQ ID NO: 70

SEQ ID NO: 72 - protein, 541 aa, Botryosphaeria parva
MDTKDVALHREQSLDEKANVTAVKEITGNEAFNEALLKEPPQPFNGASIVLYLCCLVGFFCSTMNGYDGSLLNGLLMSDDFKAYFGGSDKGIWAGIVTAMYQIGSVVALPFVGPAIDNFGRKGGMFIGALIIVIGTVINATTMFTASIGQFEAGRFILGFGVSIATAAGPMYVVEVTHPAYRGVMTALYNTFWFTGSILAAGAVRGSLDSTLKHNWVIPVWLQLLFSGLIVLFVYFLPESPRWLYVNNKREQCRDILTKYHGNGNENSIWVSLQLREYEEYLEMDGADKRWWDYRALFRDRASRYRIACNIVISIFGQWAGNAVLTYFMSALLESAGYTTEVSKANINLFYSCEQFLIAVAGALCVDKIGRRPLLLGAMLGCSLVWVGMTIATSQFDKTGSTDASKAATAMIFLFGAVFSFGVTPLQALYPVEVLSFEMRAKGMAFSSFALNAAMLLNQFAWPVSMEKIGWRTYIIFVIWDSIQAGVIYFFIPETKNRTLEELDDIFHAKNPTKASLQKRKVALDANANVVGVEPLDSDSA

SEQ ID NO: 73 - protein, 541 aa, Botryosphaeria parva; identical to SEQ ID NO: 72

SEQ ID NO: 74 - protein, 543 aa, Moniliophthora roreri
MSKESPVPSFSNDIKGSIEHVEDTIPSLEGQRKKRFSGPEERQAALEEAIKRDPGPGQWSWPTIHMCIITFIICCCSGDSGFDSTVMGGINGMHQFQEYFGMTGAGTKTSIVFGIYTIGQLCGTIPAAYFPDRFGRRFSMFFGNCILICGAVITANAKSMSMFLGGRWMTGFGCTCAATSAKSYLAEIVPPRTRGAYLGFLNSFYYVGQMSASGMMVATNLWPNELSWRLPLYIQTVPAAINALFVFTCPESPRWLASIGKHEQARKLLAKFHSQDGNINSPVIEIEMDEIKEKIEINGRDKRWWDFRPLFRTRSDRYRSYMCIIIGAFGQLSGNGLITYFLPILIKNAGIQSQSKQQTLNFINSVTSYIGALAGSFTVDRFGRRKNLFWATFTITCILAIVTGLLSQNGNATRSNAGISFIFLFMVCFSFGWTPMQALYPAEVLSYEARAKGLAFLNLVTQAASLINTFGLPVALEKIGWKTYVIFVAWDAFECVIIYFFIVETKYLTLEEIGEVFEQPNPVQYSKELYRRRATGGNDEEAH

SEQ ID NO: 75 - protein, 543 aa, Moniliophthora roreri; identical to SEQ ID NO: 74

SEQ ID NO: 76 - protein, 496 aa, Mollisia scopiformis
MFAVLTSATNGYDGSMMNGLQALPQWEASFHNPGPSTRGLLNAIMSVGSIVALPITPYIADILGRRAGVMTGCVIMIIGVVLQSIGINIQMFIAARFLIGFGVAIAHGSAPLLIAELVHPQHRAIFTTIYNSTWYFGSIVASWLTYGTFQLAGPWAWRIPSIVQAAPSCLQLIAIWMVPESPRYLIAKGKNEKALNILAKAHANGNVEDELVQIEYREIRETLQLEKEFEQNGWLEFFQTKGNRHRLIILISLGFFSQWSGNGLVSYYMNQVLQGAGVTSAKLRLEINGILNIINFLTAVTMCFFIDKFGRRPLFLFATAGMCASFCIWTICAAEFTKTAVAAAGQAEVAFIFIYYVFYNCAWSGLLVGYAVEILPYKLRAKGLTLMFLAVDLALFFNSYVNPVALAALDWKYYIVYDVWLFVELCVVFFFYIETRNTPLEEIVKYFDGEQALLGGDMATEKARAILIEEAGDHHNKTAIAHTEEVSSPNSQDGKI

SEQ ID NO: 77 - protein, 496 aa, Mollisia scopiformis; identical to SEQ ID NO: 76

SEQ ID NO: 78 - protein, 503 aa, Diplodia seriata
MFKDLGGHPTLTTALVISVCVVDSVTVAYDGSLMGSLNAMPAYSDYFTLTTATTSLNTASTFIGAILLSPFAALLINWRGRKCGIYVSALVQIAGTILQGAAQSIGMFIVGRLLIGAGSGLAQTSAATYVAETVPSKIRALALGLYFTCWAVGALLAAGVCYGTASMENSTWSWRVPSLIQAVPSILAILVLLALPESPRWLAYQGRCDEALKVLCAINGREESEPEVQIQYQEIMDNIAFEKSDGQTLGFSEVIKNKSNARRLMLAVSVPPLAMLTGSNIITFYFSTMLEQAGITDASTQLQINVILSAWQLVVALSGSLLAERIGRRMSALSSLGSCTVFFYMLGGLTSKYGNSTNASGIYGTIACIFLFLGAYSFGITPLTVMYQPEVLSYSIRATGMSISTVTSNACGLLVTFAFPFALDAIGWKTYMINATFNVFLWAFIAYFWVETKGLTLEEIDEIFDGTKHSDMPNLADFHAGRSDVIGGVEVASPVNVRVPLKL

SEQ ID NO: 79 - protein, 509 aa, Hypocrea jecorina
MKEPPKAWTKAQVLVYSFSIIAFFCSTMNGYDGSLINNLLQNPWFKAKYTVGNDGIWAGIVSSMYQIGGVVALPFVGPAIDGFGRRIGMLLGAILIVVGTIIQGLSNSQGQFMGGRFLLGFGVSIAAAAGPMYVVEINHPAYRGRVGAMYNTLWFSGAIISAGAARGGLNVGGDYSWRLITWLQALFSGLIIIFCMFLPESPRWLYVHHKKDAAKAVLTKYHGNGNPDSVWVQLQLFEYEQLLNMDGADKRWWDYRALFRSRAAVYRLLCNVTITIFGQWAGNAVLSYFLGSVLDTAGYTGTIAQANITLINNCQQFAWAILGAFLVDRVGRRPLLLFSFAACTVVWLGMTVASSEFAQSFIGNDANGDPIYSNPSASKAALAMIFIFGAVYSVGITPLQALYPVEVLSFEMRAKGMAFSSFATNAAGLLNQFAWPVSMDKIGWKTYIIFTIWDLVQTVVVYFFIPETKGRTLEELDEIFEAKNPVKTSTTKKAVAVDSHGDIVNIEKA

SEQ ID NO: 80 - protein, 509 aa, Hypocrea jecorina; identical to SEQ ID NO: 79

SEQ ID NO: 81 - protein, 533 aa, Botryosphaeria parva
MEKNATTLREESLNGPKEIKIISGTAAFEEAKLKEPPKPFAARSLILYLACLVGFLCSTANGYDGSLMNSFLETPAFLEFFHIENKGLWSGIVANMYTIGGVVALPFVGPSLDQLGRRAGMFAGATLIIIGTIIQGTTSTDASRAQFMGGRFVLGFGVSFMTAGGPILVLEISHPAYRGVMTAWYNTFWFTGSILASGTARGTIGLHGNNSWLIMTWLQLLFAGVVFLFAWILPESPRWLYTRGKRDKCREMLTYWHGHDNPDSVWVQLQLQEYEEYLEMDGSDKRWWDYRSLFRNKPSVYRLCCNLVIVVFGQWAGNAVLSYYLSSALDTAGYHDELQQKNINLILNCVQFVTALIGARTVEWFGRRPLLLFANIGCAICWVCITGSTATLAKDETNNAAGTAAVAFIFMFNIIFAFGFTPLQQLVPVEVLSFEMRAKGMAFSSFVMNLAMLMNNYAWPVSMEKIGWRTYIIFAVWDVFQAIVIYFLIPETKNRTLEELDDIFHASNPVKASLEKKRIAVDSDRHVLEVEKS

SEQ ID NO: 82 - protein, 533 aa, Botryosphaeria parv; identical to SEQ ID NO: 81

SEQ ID NO: 83 - protein, 488 aa, Dacryopinax primogenitus
MAKHKPNPLSTSMLLLYPILLVAFMNSAANGFDGNTFGGVSAIADFQARFGTNVAASDGFLAAIYILGNVIGSFVAGPAADWLGRKRGMILANIIVLIGTIVQAAAMQRRDMIAGRVVLGIGSVMLGPSATSYVVEMSYPAYRGTIVGLYNGCYFIGAIVSTWLEYGLVDDTKGEINWRIPMAMQGIPCIIVLAFVWFLPESPRWLMARGREEEAKRILIKYHGEGDPDNELVQLEMEEMHEAIDTAGSDTRWWDYRELFNTRGARHRMFLVLCVGFFGQIDLPPTSYYMPLMAQTAGIVSTKQQLLMNALQSPVMTIGTLLGVHTIDKYGRRPMLIVSSAVCSLCVLIIIICSLKQEGHPSVGLAGISFVYVFLFAFAFVWTPMQALYPSEVLAYNARAKGLGMSGLWINIVSFINTYAAPVGITNSGWKFYFLYFVIDVVGIITIYFFFIETKDRSLEEIDEIFADPHPVRTSLRKQKISVAIEKS

SEQ ID NO: 84 - protein, 488 aa, Dacryopinax primogenitus; identical to SEQ ID NO: 83

SEQ ID NO: 85 - protein, 537 aa, Cordyceps fumosorosea
MAIDEQKAHIAHDESIADEKMTGNVKTIGTGSVALAAAVASQKPRLLSKNMIQLYLIMGVGYLVSTMNGFDSSLMGSINAMKPYQESFGLAGAGSTTGIIFIIYNLGQIAAFPFCGLLADGYGRRLCIFVGCLVVVAGTAVQGSAHSLGQFVGGRFLLGFGAAIASAAGPAYTVELAHPAYRGFMAGMYNNFWWLGNILAGWTSYASNKHLQSSWAWRVPTIVQAGLPGVVMVLILFFPESPRWLIANDRAEEALAILAKYHGDGDANSAIPTLEYNEIVEMNRLGKDDNPWWDFRELWNTRAARYRLGMVVGMAFIGQWSGNNVVTYFMPEMIVQAGITDTNKQLLLNAINPIFCMLGAVYGASLLDRLGRRTMMLAGLVGALASYCMLTAFTAEAERHASLAYGVIASIYIFGIFFSWGFTPLQTLYAVECLENRTRAKGSGLNFLFLNIAMVVNTYGISVGMQQLGWKLYLVYIGWICVEMLIIYFFFVETAGKTLEQLKDVFEAKNPVKASIAKTNVELDESGRIVGVVGEGV

SEQ ID NO: 86 - protein, 532 aa, Mollisia scopiformis
MASDEKDLGAASHVNDIDNSSTDAILLATEADTTTYSPWSRSMFRLYGVLAIAYLCGCLNGFDGSLMGAINAMKPYQNYFGIGSTGSSTGIVFAIYNIGSIPAVVLTGPVNDYLGRRAGMFTGSVIIIIGTCIQAPAVNMHMFLAGRFLLGFGVSFCCVSAPCYVSELAHPKWRGTLTGLYNTCWYIGSIIASWTCYGTAFLSTNWSWRIPIWCQLLSSVVVAGGAFFLPESPRWLVGQDRVDEARAILAKYHGEGREDHPIVNLQISEMYYQIQTDATDKRWWDYSGLVKSHNARRRLICVLGMAFFGQWSGNSVSSYYFPEMMATAGIADEHTQLKLNGVFPVLCWLGAISGARMTDKIGRRPLLLWSILFSSICFAIITGTTKLAVEHNNKMASNVAITFVYLFGIVFSFGWTPLQSMYIAETLPTETRAKGTAIGNFGSSVASTVIQYSSGPAFKNITYYFYLVFVAWDLIEFVVIYFFFVETKDRTLEELDEVFSDLHPVKKSLQKRSVQTVAATVGVDVNEKSMVA

SEQ ID NO: 87 - protein, 532 aa, Mollisia scopiformis; identical to SEQ ID NO: 86

SEQ ID NO: 88 - protein, 240 aa, Diplodia seriata
MAFLPNFEATPLAPPHSSRLVIALLILSSCISSATLGYDGSMMSGLNILPAYNDYFHLTPATTALNTATVYIGQVIPCFFYGAVSDRLGRKNAMAIAAVATIAAVVLQAAAQNVAMFAVSRILVGVGNGATSIAGPVWLSECLPHRWRAWGLGFRIGYVGGLIASGITYGTGMMNSTWAWRLPSAVQGIFSVLCILILPFVPESPRWLVYQNRGEEALYALALTHSHGDQKDAATLVEFQ

SEQ ID NO: 89 - protein, 546 aa, Metarhizium robertsii
MAEKVLPVASAHINTDAKQVETRVVTGDEAFNEALLKEPPTPWSRGQLMIYLFSLVAFFNSTVNGYDGSLINNLLQNPSFIAKYNGSNSGIWAGIVSSMYQIGNIVALPFLGPVCDHFGRRAGMASGSAIIIIGTIIQGTSNAAGQFMGGRFLLGFGVSLVATGGPMYVVEINHPAYRGVVGAMYNTLWFSGSILSSGAARGAANVGGDYSWRLITWLQQILFPSLVLIFSFLLPESPRWLYVHNRKEKAKAILVKYHGNGNDSSPWVSLQLREYEECLNMNGADKRWWDYRVLFRRGNFYRLSCNLIVSAFGQLAGNAVLSYFLGSVLDSAGYTSYLAQANITLINNCVQFLCAICGALLVDRIGRRPLLLFAFTSCTVVWLGMTIAASQFAASYAGETASGVYVYTNGAASKAALAMIFLFGAAFSIGITPLQGLYIVEVLSFEMRAKGMAMSNLAVNLAGLLNQYAWSVSMKNIGWKTYIIFTVWDAISVIIIYFTLPETKGRTLEELDQIFAAKNPVKASLSKKEILVSRDGDVVDVKEASP

SEQ ID NO: 90 - protein, 546 aa, Metarhizium robertsii; identical to SEQ ID NO: 89

SEQ ID NO: 91 - protein, 535 aa, Microdochium bolleyi
MSSPTGETKPELHGEEKEFGEVRHVNAASIALAAAVAEQKPNPWSRNMISLYMIMAIGYLVSTMNGFDSSLMGAINAMDEYTTMFNLQGDDGNSTGIIFIIYNIGQIASFPFCGLLADGLGRRWCIFIGCAIVLVGTAIQTTAHERGQFIGGRFVLGFGASIASAAGPAYIVELAHPAYRGTMAGMYNNFWWLGNIIAGWTTYGTEYHLKNSWAWRAPTVVQCIMPAIVMSLIMFFPESPRWLIHHDRTEEALAIFAKYHGDGDEQSALVQLQYREIVAERAATQNPNAWWDLRELCNTKAARYRMFMVIGMSFFGQWSGNNVVSYFMPVMVEQAGIKDKSTQLLINAINPIFSMIAAIYGATLLDKLGRRFMMLAGLGGALVFYCMLTGFTAGSETNKNLSYGVIVSIYLFGVCFAWGFTPLQTLYAVECLENRTRAKGSGANFLFLNIAMVVNTYGISVGMKAIGWKLYIVYIVWIMIEMVIIYFFFVETAGKTLEEMSEIFEAPNPRKASTKKTKMGLNQAGQVVGVDEESH

SEQ ID NO: 92 - protein, 535 aa, Microdochium bolleyi; identical to SEQ ID NO: 91

SEQ ID NO: 93 - protein, 542 aa, Microdochium bolleyi
MGLFKRNKTEAETAVGPALAAASSSVVLRLPLVLPNDPRPFYKVRHLLLLNLLLLLTSLSSASIGFDGAMMNGLQTVAQWRDYFGRPTPAILGVMNAIYPIGKLVGIFPTTWLADKYGRKAPMYLGFVMLVVGAGIQGGSVHIGMFIASRFFLGFGTAFLAQPAPILVTELAYPTHRGKITAIYQTFFFFGAILAAWSTYGTLRIPSTWSWRIPSLLQGAIPAFQLALFWFVPESPRWLVAKGREAEARVILTKWHSAGDESSPLVDYEMTQIKETVQLETEALSETSYLDLVRTPANRRRTFIAVIVGFFAQWNGAGVISYYLALVLNTIGITESRDQALINGLLQVFNWLAAIFAGALMVDRLGRRTLFLASTAGMFFSYVIWTALTGVFTTTLNQNMGNAVVAFIFIYYFFYDIAWNPLLLAYPVEVFQFTLRARGVSVTYASTFIGLIIGQFVNPLAMAGLGWKYYIVFCVILACLFVVIWFTFPETKGRTLEEIAEIFDGPGAGHGAAATDEEAAAAEAKASSIENEHEDVSVPRKA

SEQ ID NO: 94 - protein, 555 aa, Scheffersomyces stipitis
MSTNSLNDSYNPSSTKEKDIVVQSEALADVAIETAFETDGYKKIFQEHPVPRWTKSRLSIYFTCLVIYLVSTTNGYDGSLLSSLITMPEFISHLNIKSASGTGIVFAIFQVGQMVATLFVWLGDFIGRRNAIFIGSVIVCLGAIITSIANNTSTFIGGRFLLSFGSGISCALSTTYLLEITSPDERSALCAIYNSLYYIGSIIATWSSYATSISYANSVLSFRIPLWLQILCPALVVIGLLVGVAPESPRFYYLTGQPDKARAFFCKYHANGDEKHPIVEYEMAQLELSLLEVPKLRVRDYFDARILFKTKSRIYRSLVCIAHSAFGQLSGNAVVGYYITNIFLELGITNPTTRLLLNGVNSILGFIFAMSGSILVGRIGRRPILLYSTTGFVISFTIIAACIAAYTNNNNQVAAKVGIAFIYIFNNVFFSFGYTPLQPLYPAEILSSEMRAKGMALFQITQGTASFINTYAAPVAMQNIKYWYYVFFVFWDTFEVIIIYLYFVETKNLTLEEIELIFESATPVKTSMIISKPGHAANEEKLRLANLKLGKNYVA

SEQ ID NO: 95 - protein, 555 aa, Scheffersomyces stipitis; identical to SEQ ID NO: 94

SEQ ID NO: 96 - protein, 360 aa, Scheffersomyces stipitis
MSSEMLSKSEVKYEQNEMEGSQEKLALKDEDSKDFYKVNEAYNEKGFPLLSRPMIPLLLTCSVVYFVSTNTGFDGSLMSSIYTQQDYLDKFNLSINSSTSTGLVFSIYNVAQICAAFFCPLIDFWGRKKLILIGCWGTVLGAIITAFAQNKETLIAGRFVLSFFTTLANTSASLYVTEIANTYNRSVVAGCYNTLWYIGSVLAAFTSYGANVNLGGTELAFRLPLGIQAVFPGLVGIFGFFIPESPRWLVGVGREKEAEEMIAKYHCNGDFSHPLLEHEMVQINESFRGNKLAQSLKILDLRPIFQNNNAYRSILVILMAFFGQFSGNNVCSYYLPTMLRNIGMTTVSTNVLMNAFYSLI

SEQ ID NO: 97 - protein, 360 aa, Scheffersomyces stipitis; identical to SEQ ID NO: 96

SEQ ID NO: 98 - protein, 533 aa, Emericella nidulans
MADEKTVQPSAEQEAVGEAEEVRVAHDALNAKYSPLTWSMFRLYLCLIIPYLCGTLNGYDGSLMGGLNAMETYLDFFNMETSGSSTGIVFALYNIGSIPAVFFTGPVNDYWGRRCGMFVGALIIVIGTCIQSPSVNRGMFLAGRFILGFGVSFCCVSAPCYVSEMAHPAWRGTITGLYNCTWYIGSILASWVVYGCSQLDNANSFRIPIWCQLISSALVVLGVWFIPESPRWLMAQDRAEDAAKILTRYHGENDPDHPLVHLQLKEMQQSIATDASDKKWWDYRELYTGHSARRRLICVLGMACFGQISGNSVTSYYLPVMLENAGIVSESRKLLFNGIYPPLSLIGAVVGARMTDTIGRRPLLIYSLLFCSVAFAIITGTSKLATDDPTNTAAANTTIAFIYLFGIVFSFGWTPLQSMYIAETLTTTTRAKGTAVGNLASSIASTIIQYSSGPAFKDIQYYFYLVFVFWDLIEIVIMYFYFPETKDRTLEELEEVFSAPNPVKRSLVKRDAATVLNTMQVEQRELVSKEAQV

SEQ ID NO: 99 - protein, 553 aa, Emericella nidulans
MGEINEEKHDISVTEGAKVATMHGMTAEKPGATTKSVFNAELFAAINETKIERWSKTSIHLYCAVCIFVSFCCACANGYDGSLMGAVFAMDHYQATFNTGMTGQKVSVVTSLYTVGSMVATPFSAVISDNFGRRKCMFVGGWVIIIGSIVIATASTLAHFIVGRFILGFGIQIMVVSAPAYAAEISPPHWRGRAVGLYNCGWFGGSIPAACVTYGCNYIDSNWSWRVPFLLQCFASVIVIISVWFIPESPRWLIAHGKEEEAIAILAKYHGNGDPNARLVRLEADEMREGIRQDGIDKRWWDYRPFLLSHNGRWRFAQVIMISIFGQWSGNGLGYFNPAIYEALGYTSSSMQLLLNLVNSIVGAIGALTAVYYCDRMPRRTVLVWGTLGCAICMAVNAGVSQPLIPQRNAGETLDPTFGRTALAFYYLFQVVFSFTYTPLQGVVPAEALETTTRAKGLALSGFLVSGTSFISQYASPIALGNISTNYFWIFVGWDVVETACWYLFGVEAQGRTLEELEYIYNQPYPVKASKKRDRVVVQQDGHVTEKISADEA

SEQ ID NO: 100 - protein, 328 aa, Bolitoglossa subpalmata
MADNTGSEMKSGSFMLEKSAKIFVAGHRGLVGSAIVRKLQDQGFTNLVLRTHSELDLTSQSDVESFFATEKPVYVILAAAKVGGIHANNTYPADFIGVNLQIQTNVIHSAYTHGVKKLLFLGSSCIYPKFAPQPIPESALLTGPLEPTNEWYAIAKIAGIKMCQAYRLQHQWDAISGMPTNLYGQNDNFHPENSHVLPALMRRFHEAKANNADEVVVWGSGSPLREFLHVDDLADACVFLMDQYSGFEHVNVGSGVEVTIKELAELVKEVVGFKGKLVWDTTKPDGTPRKLMDSSKLASLGWTPKISLKDGLSQTYEWYLENVVQKKQ

SEQ ID NO: 101 - protein, 323 aa, Arabidopsis thaliana
MAETIGSEVSSMSDKSAKIFVAGHRGLVGSAIVRKLQEQGFTNLVLKTHAELDLTRQADVESFFSQEKPVYVILAAAKVGGIHANNTYPADFIGVNLQIQTNVIHSAYEHGVKKLLFLGSSCIYPKFAPQPIPESALLTASLEPTNEWYAIAKIAGIKTCQAYRIQHGWDAISGMPTNLYGPNDNFHPENSHVLPALMRRFHEAKVNGAEEVVVWGTGSPLREFLHVDDLADACVFLLDRYSGLEHVNIGSGQEVTIRELAELVKEVVGFEGKLGWDCTKPDGTPRKLMDSSKLASLGWTPKVSLRDGLSQTYDWYLKNVCNR

SEQ ID NO: 102 - protein, 321 aa, Homo sapiens
MGEPQGSMRILVTGGSGLVGKAIQKVVADGAGLPGEDWVFVSSKDADLTDTAQTRALFEKVQPTHVIHLAAMVGGLFRNIKYNLDFWRKNVHMNDNVLHSAFEVGARKVVSCLSTCIFPDKTTYPIDETMIHNGPPHNSNFGYSYAKRMIDVQNRAYFQQYGCTFTAVIPTNVFGPHDNFNIEDGHVLPGLIHKVHLAKSSGSALTVWGTGNPRRQFIYSLDLAQLFIWVLREYNEVEPIILSVGEEDEVSIKEAAEAVVEAMDFHGEVTFDTTKSDGQFKKTASNSKLRTYLPDFRFTPFKQAVKETCAWFTDNYEQARK

SEQ ID NO: 103 - protein, 321 aa, Escherichia coli
MSKQRVFIAGHRGMVGSAIRRQLEQRGDVELVLRTRDELNLLDSRAVHDFFASERIDQVYLAAAKVGGIVANNTYPADFIYQNMMIESNIIHAAHQNDVNKLLFLGSSCIYPKLAKQPMAESELLQGTLEPTNEPYAIAKIAGIKLCESYNRQYGRDYRSVMPTNLYGPHDNFHPSNSHVIPALLRRFHEATAQNAPDVVVWGSGTPMREFLHVDDMAAASIHVMELAHEVWLENTQPMLSHINVGTGVDCTIRELAQTIAKVVGYKGRVVFDASKPDGTPRKLLDVTRLHQLGWYHEISLEAGLASTYQWFLENQDRFRG

SEQ ID NO: 104 - protein, 348 aa, Neisseria meningitidis
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 105 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTDRNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 106 - protein, 319 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPMPVPG

SEQ ID NO: 107 - protein, 300 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPNEDFRKS

SEQ ID NO: 108 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFRNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 109 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVSSHYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 110 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEAPVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 111 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTPIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 112 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNGTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 113 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLITSLNIGLDELAKSGMGEYIARTDADDIAAPDWIEKIVGEMEKDRSIIAMGAWLEVLSEEKDGNRLARHHRHGKIWKKPTRHEDIADFFPFRNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 114 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYVFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 115 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFWYDVSKLGRLAYYPEALVKYRLHANQVVSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 116 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFGNPIHNNTMIMRRSVIDGGLRYNTERDWAEDYQFNYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 117 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHLDIADFFPFRNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 118 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFRNPVHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 119 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFRNPIHNNTMIMRRSVIDGGLRYNTERDWAEDYQFWYDLSKLGRLAYYPEALVKYRLHANQVSSKYSIRQHEIAQGIQKTARNDFLQSMGFKTRFDSLEYRQIKAVAYELLEKHLPEEDFERARRFLYQCFKRTDTPPAGAWLDFAADGRMRRLFTLRQYFGILRRLLKNR

SEQ ID NO: 120 - protein, 348 aa, synthetic construct
MPSEAFRRHRAYRENKLQSLVSVLICAYNVEKYFAQSLAAVVNQTWRNLEILIVDDGSTDGTLAIAKDFQKRDSRIKILAQAQNSGLIPSLNIGLDELAKSGMGEYIARTDADDIAAPDWIEKIVGEMEKDRSIIAMGAWLEVLSEEKDGNRLARHHRHGKIWKKPTRHEDIADFFPFRNPIHNNTMIMRRSVIDGGLRYNTERDWAEDYQFWYDVSKLGRLAYYPEALVKYRLHANQVSSKYSIRQHEIAQGIQKTARNDFLQSMGFKTRFDSLEYRQIKAVAYELLEQHLPEEDFERARRFLYQCFKRTDTPPAGAWLDFAADGRMRRLFTLRQYFGILRRLLKNR

SEQ ID NO: 121 - protein, 282 aa, Pasteurella multocida
MSGEHYVISLSSAVERRQHIRNQFSQKNIPFQFFDAISPSPLLDQLVLQFFPRLADSSLTGGEKACFMSHLSLWHKCVEENLPYIVVFEDDIVLGKDADKFLIGDEWLFSRFDPEEIFIIRLETFLQKVVCESTHIAPYTHRDFLSLKSAHFGTAGYVISQGAAKFLLDIFKNISNEHIAPIDELIFNQFLVKNSFNVYQLSPAICVQELQLNNESSALQSQLELERNKFRNKKSEELKRNRKNFIEKFIYILKKPKRMLDNNKRKREESKIENDKMIIEFK

SEQ ID NO: 122 - protein, 279 aa, Neisseria gonorrhoeae
MQNHVISLASAAERRAHIAATFGSRGIPFQFFDALMPSERLERAMAELVPGLSAHPYLSGVEKACFMSHAVLWEQALDEGVPYIAVFEDDVLLGEGAEQFLAEDTWLQERFDPDSAFVVRLETMFMHVLTSPSGVADYGGRAFPLLESEHCGTAGYIISRKAMRFFLDRFAVLPPERLHPVDLMMFGNPDDREGMPVCQLNPALCAQELHYAKFHDQNSALGSLIEHDRRLNRKQQWRDSPANTFKHRLIRALTKIGREREKRRQRREQLIGKIIVPFQ